(19) United States
(12) Patent Application Publication
(10) Pub. No.: US 2025/0281479 A1
(43) Pub. Date: Sep. 11, 2025

(54) TREATMENT OF CAVERNOUS ANGIOMAS WITH A 4-SUBSTITUTED PIPERIDINE DERIVATIVE

(71) Applicant: Neurelis, Inc., San Diego, CA (US)

(72) Inventors: Adrian L. RABINOWICZ, San Diego, CA (US); Enrique J. CARRAZANA, San Diego, CA (US); Stuart MADDEN, San Diego, CA (US)

(73) Assignee: Neurelis, Inc., San Diego, CA (US)

(21) Appl. No.: 19/073,695

(22) Filed: Mar. 7, 2025

Related U.S. Application Data
(60) Provisional application No. 63/562,607, filed on Mar. 7, 2024.
(60) Provisional application No. 63/634,704, filed on Apr. 16, 2024.

(51) Int. Cl. A61K 31/4725; A61K 9/00; A61P 9/00

(52) U.S. Cl. CPC A61K 31/4725; A61K 9/0053; A61P 9/00

(57) ABSTRACT

Disclosed are methods of treating cavernous angiomas including cavernous angiomas with symptomatic hemorrhage with a 4-substituted piperidine derivative. Also disclosed are methods wherein administering the therapeutically effective amount of a 4-substituted piperidine derivative results in a Cmax of between about 3 ng/ml and about 60 ng/ml; and wherein a Tmax is achieved of between about 0.5 hours and about 1 hour of administering of a 4-substituted piperidine derivative.

### CROSS-REFERENCE TO RELATED APPLICATIONS

This application claims priority to U.S. Provisional Application No. 63/562,607 filed Mar. 7, 2024, and U.S. Provisional Application No. 63/634,704 filed Apr. 16, 2024, each of which are hereby incorporated by reference in their entirety.

### SUMMARY OF THE INVENTION

The present invention relates to a method of treating cavernous angioma lesions with symptomatic hemorrhage in a subject comprising: administering to the subject a therapeutically effective amount of a compound of formula (II):

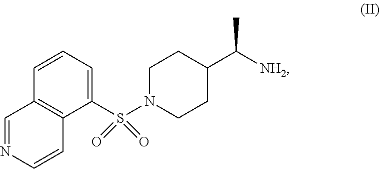 [SMILES: C[C@@H](N)C1CCN(S(=O)(=O)c2cccc3cnccc23)CC1]

or a pharmaceutically acceptable salt thereof;
wherein administering the therapeutically effective amount of the compound of formula (II), or a pharmaceutically acceptable salt thereof results in a Cmax of between about 3 ng/ml and about 60 ng/ml; and
wherein a Tmax is achieved of between about 0.5 hours and about 1 hour of administering the compound of formula (II), or a pharmaceutically acceptable salt thereof.

In some embodiments, the salt is an adipate salt of the compound of formula (II) or a pharmaceutically acceptable salt thereof.

In some embodiments, the therapeutically effective amount of the compound of formula (II), or a pharmaceutically acceptable salt thereof is from about 25 mg to about 250 mg.

In some embodiments, the therapeutically effective amount of the compound of formula (II), or a pharmaceutically acceptable salt thereof is an amount sufficient to result in a plasma concentration of between about 0.1 ng/ml and about 2000 ng/ml of a compound of Formula (IV):

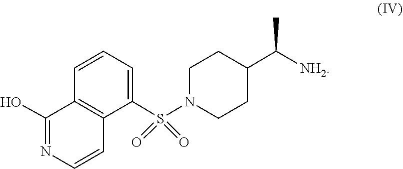 [SMILES: C[C@@H](N)C1CCN(S(=O)(=O)c2cccc3c(O)nccc23)CC1]

In some embodiments, the plasma concentration of the compound of Formula (IV) is maintained for at least 48 hours following administration of the compound of formula (II), or a pharmaceutically acceptable salt thereof. In some embodiments, the therapeutically effective amount of the compound of formula (II), or a pharmaceutically acceptable salt thereof is an amount sufficient to result in a Cmax of between about 50 ng/ml and about 1600 ng/ml of a compound of Formula (IV):

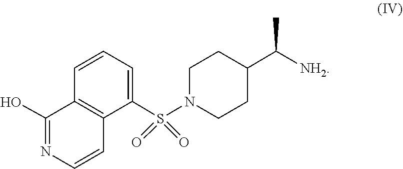 [SMILES: C[C@@H](N)C1CCN(S(=O)(=O)c2cccc3c(O)nccc23)CC1]

In some embodiments, the therapeutically effective amount of the compound of formula (II), or a pharmaceutically acceptable salt thereof is an amount sufficient to achieve a Tmax for a compound of Formula (IV):

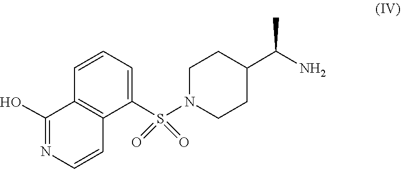 [SMILES: C[C@@H](N)C1CCN(S(=O)(=O)c2cccc3c(O)nccc23)CC1]

between about 1 hour and about 1.75 hours of administering the compound of formula (II), or a pharmaceutically acceptable salt thereof.

In some embodiments, the compound of formula (II) or a pharmaceutically acceptable salt thereof is administered orally. In some embodiments, the subject is fasted when administering to the subject the therapeutically effective amount of the compound of formula (II), or a pharmaceutically acceptable salt thereof. In some embodiments, the subject is fed when administering to the subject the therapeutically effective amount of the compound of formula (II), or a pharmaceutically acceptable salt thereof. In some embodiments, the compound of formula (II), or a pharmaceutically acceptable salt thereof is administered parenterally. In some embodiments, the compound of formula (II), or a pharmaceutically acceptable salt thereof is administered once a day. In some embodiments, the compound of formula (II), or a pharmaceutically acceptable salt thereof is administered twice a day. In some embodiments, the compound of formula (II), or a pharmaceutically acceptable salt thereof is administered three times a day.

In some embodiments, wherein the subject is diagnosed with a sporadic cavernous angioma lesion. In some embodiments, the subject is diagnosed with a familial cavernous angioma lesion caused by germline loss-of-function mutations in a gene selected from the group consisting of CCM1 (KRIT1), CCM2 (malcavernin, MGC4607), or CCM3 (PDCD10). In some embodiments, the subject has multiple cavernous angioma lesions. In some embodiments, the cavernous angioma lesion has not been resected prior to administration of the compound of formula (II), or a pharmaceutically acceptable salt thereof. In some embodiments, the cavernous angioma lesion occurs in the brainstem. In some embodiments, the cavernous angioma lesion is a Stage 1 lesion. In some embodiments, the cavernous angioma lesion is a Stage 2 lesion. In some embodiments, the subject has had a symptomatic first hemorrhage prior to administration of the compound of formula (II), or a pharmaceutically acceptable salt thereof. In some embodiments, the subject has had at least one subsequent hemorrhage/rebleed following a symptomatic first hemorrhage prior to administration of the compound of formula (II), or a pharmaceutically acceptable salt thereof. In some embodiments, administering to the subject the therapeutically effective amount of the compound of formula (II), or a pharmaceutically acceptable salt thereof results in a decrease in the number of cavernous angioma lesions. In some embodiments, administering to the subject the therapeutically effective amount of the compound of formula (II), or a pharmaceutically acceptable salt thereof results in a decrease in the number of subsequent hemorrhages/rebleeds. In some embodiments, administering to the subject the therapeutically effective amount of the compound of formula (II), or a pharmaceutically acceptable salt thereof results in an improvement in blood brain barrier permeability measured by MRI imaging using the Dynamic Contrast Enhanced Quantitative Perfusion (DCEQP), Quantitative Susceptibility Mapping (QSM) or a combination thereof. In some embodiments, administering to the subject the therapeutically effective amount of the compound of formula (II), or a pharmaceutically acceptable salt thereof results in increased lesion stability measured by Quantitative Susceptibility Mapping (QSM). In some embodiments, a decrease of about 6% less in mean lesional Quantitative Susceptibility Mapping (QSM) is indicative of increased lesional stability. In some embodiments, increased lesional stability is indicative of a lack of growth or symptomatic hemorrhages/rebleeds. In some embodiments, administering to the subject the therapeutically effective amount of the compound of formula (II), or a pharmaceutically acceptable salt thereof results in an improvement in subjects' modified Rankin Scale, Mini-Mental State Examination, Euro-Quality of Life [QoL]5D, Euro-QoL, Visual Analogue Scale, Neuro-QoL, or any combination thereof. In some embodiments, administering to the subject the therapeutically effective amount of the compound of formula (II), or a pharmaceutically acceptable salt thereof results in a decrease in one or more symptoms of a symptomatic hemorrhage selected from double vision, facial droop, balance problems, difficulty swallowing, pupil and vision changes, nausea, projectile vomiting, headache, confusion, impaired consciousness and combinations thereof.

The present invention also relates to a method of treating cavernous angioma lesions in a subject comprising: administering to the subject the therapeutically effective amount of the compound of formula (II):

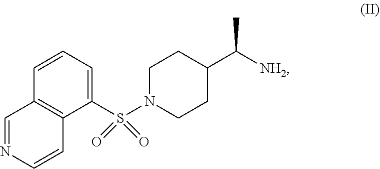 [SMILES: C[C@@H](N)C1CCN(S(=O)(=O)c2cccc3cnccc23)CC1]

or a pharmaceutically acceptable salt thereof;
wherein administering the therapeutically effective amount of the compound of formula (II), or a pharmaceutically acceptable salt thereof results in a Cmax of between about 3 ng/ml and about 60 ng/ml; and
wherein a Tmax is achieved of between about 0.5 hours and about 1 hour of administering the compound of formula (II), or a pharmaceutically acceptable salt thereof.

In some embodiments, the salt is an adipate salt of the compound of formula (II), or a pharmaceutically acceptable salt thereof. In some embodiments, the therapeutically effective amount of the compound of formula (II) is from about 25 mg to about 250 mg.

In some embodiments, the therapeutically effective amount of the compound of formula (II) is an amount sufficient to result in a plasma concentration of between about 0.1 ng/ml and about 2000 ng/ml of a compound of Formula (IV):

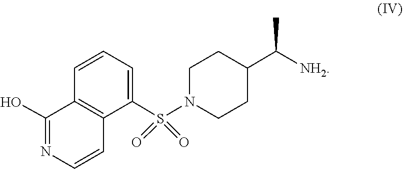 [SMILES: C[C@@H](N)C1CCN(S(=O)(=O)c2cccc3c(O)nccc23)CC1]

In some embodiments, the plasma concentration of the compound of Formula (IV) is maintained for at least 48 hours following administration of the compound of formula (II), or a pharmaceutically acceptable salt thereof.

In some embodiments, the therapeutically effective amount of the compound of formula (II), or a pharmaceutically acceptable salt thereof is an amount sufficient to result in a Cmax of between about 50 ng/ml and about 1600 ng/ml of a compound of Formula (IV):

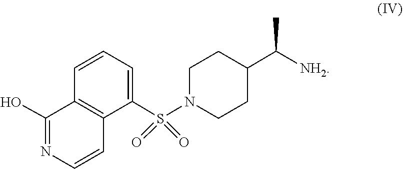 [SMILES: C[C@@H](N)C1CCN(S(=O)(=O)c2cccc3c(O)nccc23)CC1]

In some embodiments, the therapeutically effective amount of the compound of formula (II), or a pharmaceutically acceptable salt thereof is an amount sufficient to achieve a Tmax of a compound of Formula (IV):

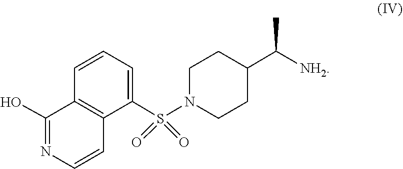 [SMILES: C[C@@H](N)C1CCN(S(=O)(=O)c2cccc3c(O)nccc23)CC1]

between about 1 hour and about 1.75 hours of administering the compound of formula (II), or a pharmaceutically acceptable salt thereof.

In some embodiments, the compound of formula (II), or a pharmaceutically acceptable salt thereof is administered orally. In some embodiments, the subject is fasted when administering to the subject the therapeutically effective amount of the compound of formula (II), or a pharmaceutically acceptable salt thereof. In some embodiments, the subject is fed when administering to the subject the therapeutically effective amount of the compound of formula (II), or a pharmaceutically acceptable salt thereof. In some embodiments, the compound of formula (II), or a pharmaceutically acceptable salt thereof is administered once a day. In some embodiments, the compound of formula (II), or a pharmaceutically acceptable salt thereof is administered twice a day. In some embodiments, the compound of formula (II), or a pharmaceutically acceptable salt thereof is administered three times a day.

In some embodiments, the subject is diagnosed with a sporadic cavernous angioma lesion. In some embodiments, the subject is diagnosed with a familial cavernous angioma lesion caused by germline loss-of-function mutations in a gene selected from the group consisting of CCM1 (KRIT1), CCM2 (malcavernin, MGC4607), CCM3 (PDCD10) or any combination thereof. In some embodiments, the subject has multiple cavernous angioma lesions. In some embodiments, the cavernous angioma lesion has not been resected prior to administration of the compound of formula (II) or a pharmaceutically acceptable salt thereof. In some embodiments, the cavernous angioma lesion occurs in the brainstem. In some embodiments, the cavernous angioma lesion is a Stage 1 lesion. In some embodiments, the cavernous angioma lesion is a Stage 2 lesion. In some embodiments, administering to the subject the therapeutically effective amount of the compound of formula (II), or a pharmaceutically acceptable salt thereof results in a decrease in the number of cavernous angioma lesions. In some embodiments, administering to the subject the therapeutically effective amount of the compound of formula (II), or a pharmaceutically acceptable salt thereof results in an improvement in blood brain barrier permeability measured by MRI imaging using the Dynamic Contrast Enhanced Quantitative Perfusion (DCEQP), Quantitative Susceptibility Mapping (QSM) or a combination thereof. In some embodiments, administering to the subject the therapeutically effective amount of the compound of formula (II), or a pharmaceutically acceptable salt thereof results in increased lesion stability measured by Quantitative Susceptibility Mapping (QSM). In some embodiments, a decrease of about 6% less in mean lesional Quantitative Susceptibility Mapping (QSM) is indicative of increased lesional stability. In some embodiments, increased lesional stability is indicative of a lack of growth, symptomatic hemorrhages/rebleeds or a combination thereof. In some embodiments, administering to the subject the therapeutically effective amount of the compound of formula (II), or a pharmaceutically acceptable salt thereof results in an improvement in subjects' modified Rankin Scale, Mini-Mental State Examination, Euro-Quality of Life [QoL]5D, Euro-QoL, Visual Analogue Scale, Neuro-QoL, or any combination thereof. In some embodiments, administering to the subject the therapeutically effective amount of the compound of formula (II), or a pharmaceutically acceptable salt thereof results in a decrease in one or more symptoms of cavernous angioma lesions. In some embodiments, the one or more symptoms of cavernous angioma lesions are selected from seizures, neurological deficits, headache, fatigue, weakness, tingling, numbness, pain, bladder issues, bowel issues, respiratory distress, and a combination thereof. In some embodiments, wherein the seizure is selected from focal onset seizures, generalized onset seizures, and a combination thereof. In some embodiments, the neurological deficits are selected from ataxia, speech and swallowing difficulties, facial paralysis, vision and auditory problems, diaphragmatic spasms, and breathing difficulties, and a combination thereof.

### BRIEF DESCRIPTION OF THE FIGURES

FIG. 1 shows the impact of the compound of formula (II) (NRL-1049) compared to vehicle on a middle cerebral artery occlusion (MCAO) stroke model in mice (a) on pMLC (ROCK activity) and (b) on IBA1 (activated microglia).

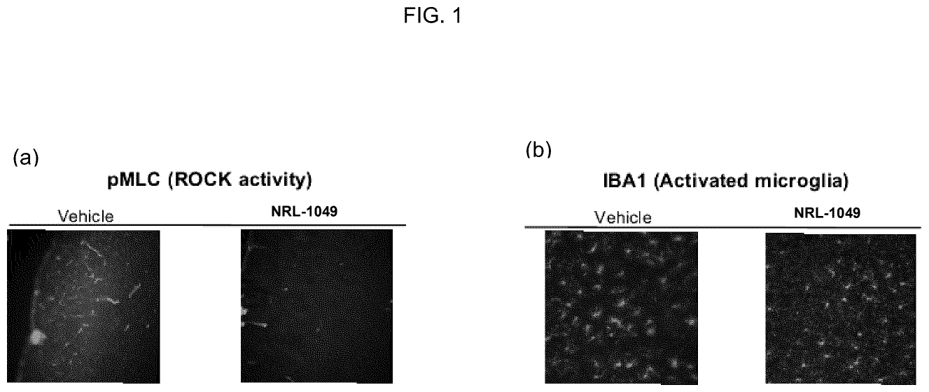

FIG. 2 shows the impact of the compound of formula (II) (NRL-1049) compared to vehicle in a cryo-freeze model in mice.

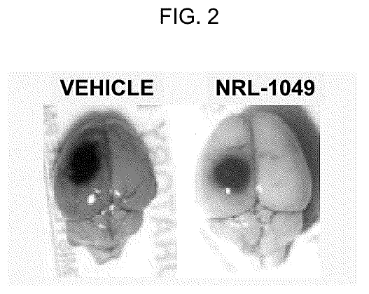

FIG. 3 shows the effect of the compound of formula (II) (NRL-1049) versus vehicle on (a) seizure-free survival post injury, (b) blood brain barrier integrity using Evans blue dye, and (c) water content.

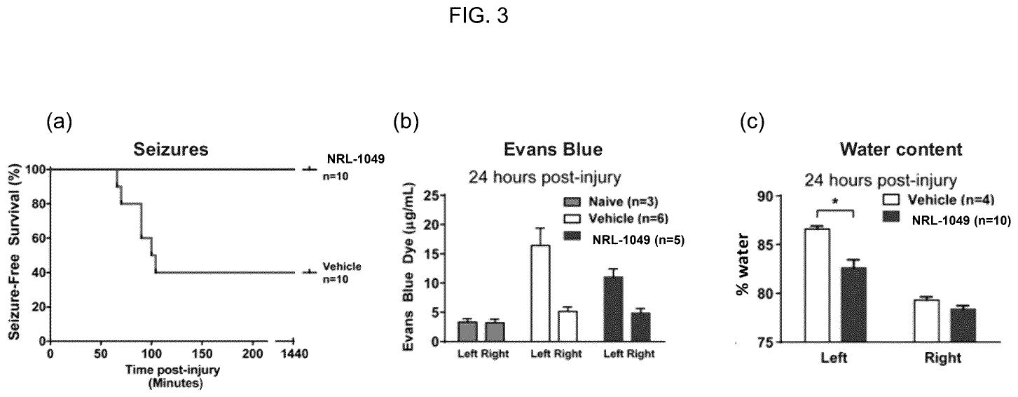

FIG. 4 shows the effect of the compound of formula (II) (NRL-1049) versus placebo in the Ccm3+/−Trp53−/− mouse model (red areas are lesions).

FIG. 5 shows the integrated density of non-heme iron per stage 2 lesion and lesional volume/brain volume per mouse with various doses of compound of formula (II) (NRL-1049) (1, 10, and 100 mg/kg) versus placebo.

FIG. 6 shows lesional volume/brain volume per mouse and the percentage of mice with lesions with various doses of the compound of formula (II) (NRL-1049) (1, 10, and 100 mg/kg/day) versus placebo.

FIG. 7 shows the PK profile for the compound of formula (II) (NRL-1049) and its metabolite, the compound of formula (IV) (NRL-2017) following a single dose of 25, 50, 150 or 250 mg of the compound of formula (II) (NRL-1049).

FIG. 8 shows the mean (+SD) Plasma Concentration-Time Profiles of NRL-1049 Under Fasted Condition—Semi-Log Scale.

FIG. 9 shows the mean (+SD) Plasma Concentration-Time Profiles of NRL-2017 Under Fasted Condition—Semi-Log Scale.

FIG. 10 shows the mean (+SD) Plasma Concentration-Time Profiles of NRL-1049 Under Fasted and Fed Conditions—Semi-Log Scale.

FIG. 11 shows the mean (+SD) Plasma Concentration-Time Profiles of NRL-2017 Under Fasted and Fed Conditions—Semi-Log Scale.

### DETAILED DESCRIPTION

This disclosure is not limited to the particular systems, devices and methods described, as these may vary. The terminology used in the description is for the purpose of describing the particular versions or embodiments only and is not intended to limit the scope. Such aspects of the disclosure be embodied in many different forms; rather, these embodiments are provided so that this disclosure will be thorough and complete, and will fully convey its scope to those skilled in the art.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

Where a range of values is provided, it is intended that each intervening value between the upper and lower limit of that range and any other stated or intervening value in that stated range is encompassed within the disclosure. For example, if a range of 1 mg/kg to 10 mg/kg μm is stated, it is intended that 2 mg/kg, 3 mg/kg, 4 mg/kg, 5 mg/kg, 6 mg/kg, 7 mg/kg, 8 mg/kg, and 9 mg/kg are also explicitly disclosed, as well as the range of values greater than or equal to 1 mg/kg and the range of values less than or equal to 10 mg/kg.

The term “subject” may be taken to mean any living organism which may be treated with compounds of the present invention. As such, the term “subject” may include, but is not limited to, any non-human mammal, primate or human. In some embodiments, the subject is a mammal, such as mice, rats, other rodents, rabbits, dogs, cats, swine, cattle, sheep, horses, primates, or humans. In some embodiments, the subject is an adult, child or infant. In some embodiments, the subject is a human.

The terms “administer,” “administering” or “administration” as used herein refer to either directly administering a compound (also referred to as an agent of interest) or pharmaceutically acceptable salt of the compound (agent of interest) or a composition to a subject.

The term “treat,” “treated,” or “treating” as used herein refers to both therapeutic treatment and prophylactic or preventative measures, wherein the object is to reduce the frequency of, or delay the onset of, symptoms of a medical condition, enhance the texture, appearance, color, sensation, or hydration of the intended tissue treatment area of the tissue surface in a subject relative to a subject not receiving the compound or composition, or to otherwise obtain beneficial or desired clinical results. For the purposes of this invention, beneficial or desired clinical results include, but are not limited to, reversal, reduction, or alleviation of symptoms of a condition; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

As used herein, the term “therapeutic” or “therapeutic agent” or “pharmaceutically active agent” means an agent utilized to treat, combat, ameliorate, prevent or improve an unwanted condition or disease of a patient.

The term “composition” as used herein refers to a combination or a mixture of two or more different ingredients, components, or substances.

The phrase “therapeutically effective” is intended to qualify the amount of active ingredients (i.e, the compounds or derivatives thereof) used in the treatment of a disease or disorder or on the effecting of a clinical endpoint.

A “therapeutically effective amount” or “effective amount” of a compound or composition is a predetermined amount calculated to achieve the desired effect, i.e., to inhibit, block, or reverse the activation, migration, or proliferation of cells. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. The compounds are effective over a wide dosage range. However, it will be understood that the effective amount administered will be determined by the physician in the light of the relevant circumstances including the condition to be treated, the choice of compound to be administered, and the chosen route of administration, and therefore the above dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

The phrase “pharmaceutically acceptable salt(s)”, as used herein, includes those salts of compounds of the disclosure that are safe and effective for use in mammals and that possess the desired biological activity. Pharmaceutically acceptable salts include salts of acidic or basic groups present in compounds of the disclosure or in compounds identified pursuant to the methods of the disclosure. Pharmaceutically acceptable acid addition salts include, but are not limited to, adipate, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the disclosure can form pharmaceutically acceptable salts with various amino acids. Suitable base salts include, but are not limited to, aluminum, calcium, lithium, magnesium, potassium, sodium, zinc, iron and diethanolamine salts. Pharmaceutically acceptable base addition salts are also formed with amines, such as organic amines. Examples of suitable amines are N,N′-dibenzylethylenediamine, chloroprocaine, choline, diethanolamine, dicyclohexylamine, ethylenediamine, N-methylglucamine, and procaine.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason. Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

Rho Signaling and its Effect on the Cerebral Vasculature

Rho kinases control the activity of rho, one of five groups of small GTP binding proteins that have GTPase activity. Rho-associated, coiled-coil-containing protein kinase (ROCK) regulates cell cytoskeleton organization, adhesion, motility, and cycle, and hence rho kinases regulate these ROCK activities. There are two isoforms of rho kinase, ROCK 1 and ROCK 2. ROCK 1 has widespread tissue distribution (but less in brain and skeletal muscle). ROCK 2 is expressed in brain, heart, and lung, but is relatively low in liver, spleen, kidney, and testes. Both ROCK 1 and ROCK 2 are activated by rho.

Overactivation of rho signaling leads to a disruption in the integrity of the cerebral vasculature. This overactivation causes cytoskeletal changes within the endothelial and smooth muscle layers of the cerebral vessel walls that disrupt vascular integrity. In particular, stress fiber formation within endothelial cells upon rho activation leads to cell contraction and thus gap formation in the endothelial barrier; this is exacerbated by the disruption of intra-endothelial junctions upon rho overactivation. Stress fiber formation within smooth muscle cells and a reduction in the secretion of vasoactive factors combine to cause vascular contraction and resulting hyper-tension/hemodynamic shear stress. Abnormalities in endothelial remodeling lead to the formation of atypical vascular structures more prone to leakage or rupture. Rho kinase activation also increases invasion by inflammatory leukocytes and resulting wall degradation and may result in depletion of the smooth muscle-like pericytes that provide structural support to the capillary wall. These rho kinase-based disruptions in vascular integrity appear to underlie the formation/growth/rupture of aneurysms in cerebral arteries and malformations in cerebral capillaries.

### Cavernous Angioma

On a molecular level, altered remodeling resulting in, e.g., Cavernous Angioma (also known as a cerebral cavernous malformation (CCM)) formation, stems directly from the overactivation of rho signaling in endothelial cells upon knockdown of CCM1/CCM2/CCM3 proteins. Wildtype CCM proteins play a critical role in the downregulation of rho signaling: CCM2 has been shown to bind the ubiquitin ligase Smurf1 and localize Smurf1 in a manner that facilitates rho degradation. Loss of endothelial cell expression of CCM1, CCM2, or CCM3 results in the activation of RhoA and activation of rho kinase. This increase in rho and ROCK activity affects cytoskeletal dynamics and results in the loss of endothelial tube cell formation (Borikova et al. (2010) JBC 285:11760-11764). ROCK 1 and ROCK 2 affect cytoskeletal dynamics differently. For example, knockdown of expression of ROCK 1 in glioblastoma cells prevents cell migration, whereas knockdown of ROCK 2 enhances cell proliferation.

The resulting overactivation of the rho signaling pathway upon CCM protein knockdown disrupts the dynamic process of blood vessel formation/repair, as rho signaling directly regulates the cytoskeletal changes underlying endothelial remodeling and migration during angiogenesis and rho kinase overactivation is associated with pathological angiogenesis. The formation and growth of the dilated and clustered phenotype characteristic of the CCM malformation appears to arise from endothelial instability during vascular remodeling. In particular, CCM1/CCM2/CCM3 protein loss results in impaired angiogenesis, as indicated by defects in vessel-like tube formation and loss of endothelial cell invasion of the extracellular matrix. Angiogenesis occurs in sequential steps that include cell proliferation, cell migration, cell shape rearrangements, and endothelial tube formation. Rho and ROCK 1 and ROCK 2 are important for each step.

Hemorrhage from cavernous angioma lesions stems from a disruption in endothelial barrier integrity characterized by (1) increased intracellular actin stress fibers and (2) decreased intra-endothelial junctions. Both of these of hallmarks of vascular leakage are characteristic of dysregulated rho signaling. Rho overactivation increases intra-endothelial contractility via overactivation of rho kinase, which leads to the phosphorylation and inactivation of myosin light chain phosphatase and so activation of myosin II and the formation of intracellular stress fibers. When the centripetal tension from the contraction of these stress fibers outbalances the adhesive forces at cell-cell junctions, endothelial cells contract and pull apart from one another, forming gaps in the endothelial barrier. An imbalance in the rho signaling equilibrium also increases permeability at intra-endothelial junctions; rho signaling regulates the formation of tight and adherens junctions and rho kinase overactivation has been shown to promote the adherens junction disruption and tight junction opening necessary for blood leakage through the endothelial barrier.

Treatment with rho inhibitors or ROCK 2 inhibitors such as the compound of Formula (II) can rescue these endothelial abnormalities and restore vascular stability. Thus, cavernous angioma lesion growth/repair is intimately linked to a disruption in the rho equilibrium underlying abnormal vascular remodeling, and inhibition of rho signaling represents a potent mechanism for preventing or slowing lesion formation/expansion.

The present invention relates to a method of treating cavernous angioma lesions in a subject comprising: administering to the subject a therapeutically effective amount of a compound of formula (I):

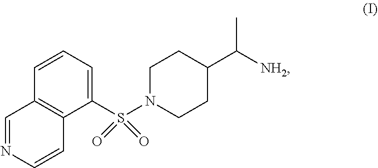 [SMILES: CC(N)C1CCN(S(=O)(=O)c2cccc3cnccc23)CC1]

or a pharmaceutically acceptable salt thereof. In some embodiments, a racemic mixture made up of the (R) and (S) enantiomers the compound of formula (I) can be used for treatment or may be separated into its enantiomers. In some embodiments, the (R) enantiomer of the compound of formula (I) can be used for treatment. In some embodiments, the (S) enantiomer of the compound of formula (I) can be used for treatment.

The present invention also relates to a method of treating cavernous angioma lesions in a subject comprising: administering to the subject the therapeutically effective amount of a compound of formula (II):

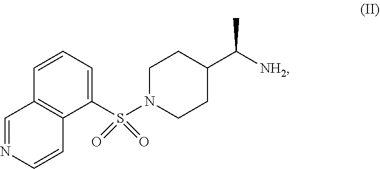 [SMILES: C[C@@H](N)C1CCN(S(=O)(=O)c2cccc3cnccc23)CC1]

or a pharmaceutically acceptable salt thereof. The compound of formula (II) is also known as NRL-1049.

In some embodiments, the pharmaceutically acceptable salt is an adipate salt of the compound of formula (II). In some embodiments, the adipate salt of the compound of formula (II) is a compound formula (III):

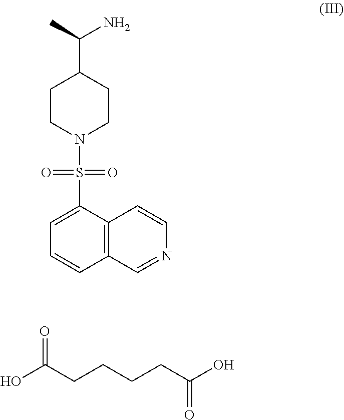 [SMILES: C[C@@H](N)C1CCN(S(=O)(=O)c2cccc3cnccc23)CC1 ; O=C(O)CCCCC(=O)O]

The present disclosure is also directed to methods of using the hydroxy metabolite of a compound of formula (II):

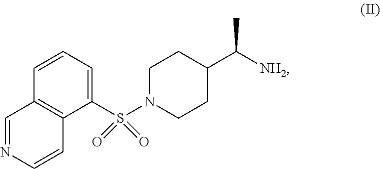 [SMILES: C[C@@H](N)C1CCN(S(=O)(=O)c2cccc3cnccc23)CC1]

or a pharmaceutically acceptable salt thereof. In one aspect, the hydroxy metabolite of the compound of formula (II) is the compound of Formula (IV):

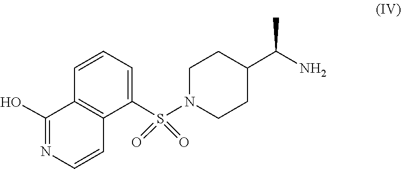 [SMILES: C[C@@H](N)C1CCN(S(=O)(=O)c2cccc3c(O)nccc23)CC1]

or a pharmaceutically acceptable salt thereof. In some embodiments, the pharmaceutically acceptable salt is an adipate salt of the compound of formula (IV). The compound of formula (IV) is also known as NRL-2017.

The present disclosure is also directed to methods of using a compound of Formula (V):

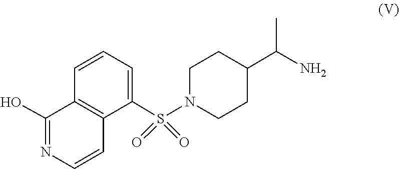 [SMILES: CC(N)C1CCN(S(=O)(=O)c2cccc3c(O)nccc23)CC1]

or a pharmaceutically acceptable salt thereof. In some embodiments, the pharmaceutically acceptable salt is an adipate salt of the compound of formula (V). In some embodiments, a racemic mixture made up of the (R) and (S) enantiomers the compound of formula (V) can be used for treatment or may be separated into its enantiomers. In some embodiments, the (R) enantiomer of the compound of formula (V) can be used for treatment. In some embodiments, the (S) enantiomer of the compound of formula (V) can be used for treatment.

The compounds disclosed herein are known in the art and can be synthesized by known methods or commercially obtained from available vendors.

A careful balance of rho activity is required to regulate the actin cytoskeleton for cell shape and motility, and to preserve junctional contacts between cells. Both rho kinase activation and rho kinase inhibition can affect the ability of endothelial cells to form tubes, a critical process required for blood vessel formation. The formation of blood vessels is a highly orchestrated process that requires the participation of other cell types, such as pericytes, and signaling by growth factors, such as VEGF. With regard to cavernous angioma formation, the imbalance of any of these signals may play a role in the initiation of growth of a cavernous angioma. Too much activated rho and rho kinase, as in cavernous angioma, causes loss of cell-cell junction integrity and vascular leakiness because it induces stress fiber formation and contraction, causing the cells to pull apart. This lets solutes and blood cells seep between the cells, which is observed in brains of patients with cavernous angioma. No rho activity (i.e., with an overly potent ROCK ½ inhibitor) prevents proliferation and migration of endothelial cells that is required for capillary formation.

The balance of inactive rho to active rho is important to cell function, and the ratio of ROCK 1 to ROCK 2 defines efficacy of ROCK inhibitors and risk/benefit ratios. In fibroblasts, treatment of cells isolated from ROCK 1 or ROCK 2 knock-out mice with the pan-ROCK inhibitor Y27632 had differential effects, showing functional differences between ROCK 1 and ROCK 2 in regulating the cytoskeleton of fibroblasts (Shi et al. (2013) Cell Death and Disease 4: e483). In neurons, inhibition of ROCK 2 has a more potent effect on promoting neurite outgrowth than ROCK 1, and ROCK 2 is more highly expressed in neurons (U.S. Pat. No. 7,572,913). In endothelial cells, the cell type affected in CCM, there is more ROCK 2 than ROCK 1, and ROCK 1 and ROCK 2 inhibitors have differential effects on endothelial cells, as shown below.

The present invention relates to a method of treating cavernous angioma lesions in a subject comprising: administering to the subject a pharmaceutical composition comprising a therapeutically effective amount of a compound of formula (I):

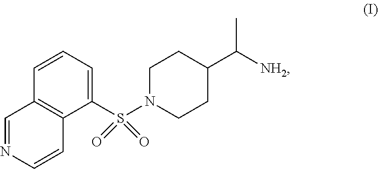 [SMILES: CC(N)C1CCN(S(=O)(=O)c2cccc3cnccc23)CC1]

or a pharmaceutically acceptable salt thereof, and a pharmaceutically acceptable carrier.

The present invention relates to a method of treating cavernous angioma lesions in a subject comprising: administering to the subject a pharmaceutical composition comprising a therapeutically effective amount of a compound of formula (II):

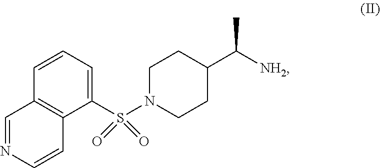 [SMILES: C[C@@H](N)C1CCN(S(=O)(=O)c2cccc3cnccc23)CC1]

or a pharmaceutically acceptable salt thereof.

The present invention relates to a method of treating cavernous angioma lesions in a subject comprising: administering to the subject a pharmaceutical composition comprising a therapeutically effective amount of a compound of formula (II):

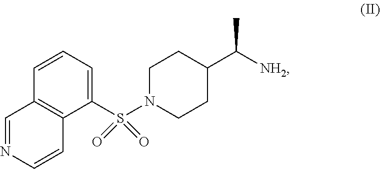 [SMILES: C[C@@H](N)C1CCN(S(=O)(=O)c2cccc3cnccc23)CC1]

or a pharmaceutically acceptable salt thereof, and a pharmaceutically acceptable carrier.

The present invention relates to a method of treating cavernous angioma lesions with symptomatic hemorrhage in a subject comprising: administering to the subject a therapeutically effective amount of a compound of formula (II):

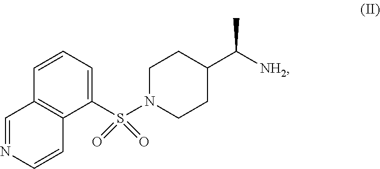 [SMILES: C[C@@H](N)C1CCN(S(=O)(=O)c2cccc3cnccc23)CC1]

or a pharmaceutically acceptable salt thereof;
wherein administering the therapeutically effective amount of the compound of formula (II), or a pharmaceutically acceptable salt thereof results in a Cmax of between about 3 ng/ml and about 60 ng/ml; and wherein a Tmax is achieved of between about 0.5 hours and about 1 hour of administering the compound of formula (II), or a pharmaceutically acceptable salt thereof.

The present invention relates to a method of treating cavernous angioma lesions with symptomatic hemorrhage in a subject comprising: administering to the subject a therapeutically effective amount of a compound of formula (II):

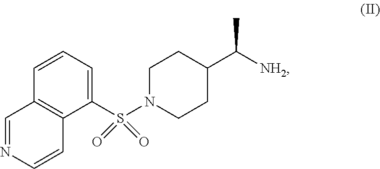 [SMILES: C[C@@H](N)C1CCN(S(=O)(=O)c2cccc3cnccc23)CC1]

or a pharmaceutically acceptable salt thereof;
wherein administering the therapeutically effective amount of the compound of formula (II), or a pharmaceutically acceptable salt thereof results in a Cmax of between 3.659 ng/ml and 58.006 ng/ml; and
wherein a Tmax is achieved of between 0.5 hours and 0.88 hours of administering the compound of formula (II), or a pharmaceutically acceptable salt thereof.

In some embodiments, the salt is an adipate salt of the compound of formula (II) or a pharmaceutically acceptable salt thereof.

In some embodiments, the therapeutically effective amount of the compound of formula (II), or a pharmaceutically acceptable salt thereof is from about 25 mg to about 250 mg.

In some embodiments, the therapeutically effective amount of the compound of formula (II), or a pharmaceutically acceptable salt thereof is an amount sufficient to result in a plasma concentration of between about 0.1 ng/ml and about 2000 ng/ml of a compound of Formula (IV):

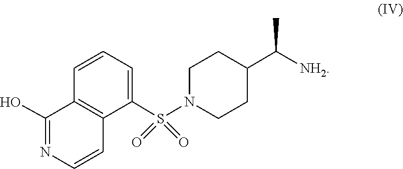 [SMILES: C[C@@H](N)C1CCN(S(=O)(=O)c2cccc3c(O)nccc23)CC1]

In some embodiments, the plasma concentration of the compound of Formula (IV) is maintained for at least 48 hours following administration of the compound of formula (II), or a pharmaceutically acceptable salt thereof. In some embodiments, the therapeutically effective amount of the compound of formula (II), or a pharmaceutically acceptable salt thereof is an amount sufficient to result in a Cmax of between about 50 ng/ml and about 1600 ng/ml of a compound of Formula (IV):

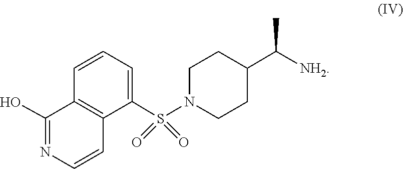 [SMILES: C[C@@H](N)C1CCN(S(=O)(=O)c2cccc3c(O)nccc23)CC1]

In some embodiments, the therapeutically effective amount of the compound of formula (II), or a pharmaceutically acceptable salt thereof is an amount sufficient to result in a Cmax of between 54.23 ng/ml and 1524.23 ng/ml of a compound of Formula (IV):

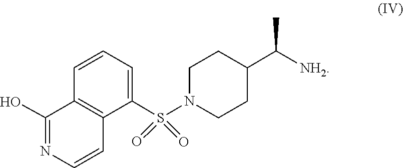 [SMILES: C[C@@H](N)C1CCN(S(=O)(=O)c2cccc3c(O)nccc23)CC1]

In some embodiments, the therapeutically effective amount of the compound of formula (II), or a pharmaceutically acceptable salt thereof is an amount sufficient to achieve a Tmax for a compound of Formula (IV):

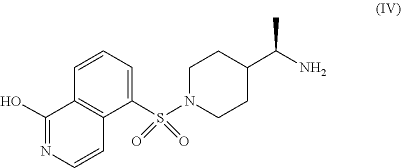 [SMILES: C[C@@H](N)C1CCN(S(=O)(=O)c2cccc3c(O)nccc23)CC1]

between about 1 hour and about 1.75 hours of administering the compound of formula (II), or a pharmaceutically acceptable salt thereof.

In some embodiments, the therapeutically effective amount of the compound of formula (II), or a pharmaceutically acceptable salt thereof is an amount sufficient to achieve a Tmax for a compound of Formula (IV):

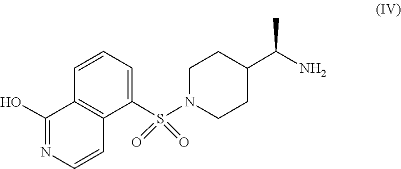 [SMILES: C[C@@H](N)C1CCN(S(=O)(=O)c2cccc3c(O)nccc23)CC1]

between 1 hour and 1.75 hours of administering the compound of formula (II), or a pharmaceutically acceptable salt thereof.

In some embodiments, the compound of formula (II), or a pharmaceutically acceptable salt thereof is administered by any suitable route of administration. In some embodiments, the compound of formula (II), or a pharmaceutically acceptable salt thereof is administered by a route of administration selected from the group consisting of oral, rectal, topical, buccal, sub-lingual, vaginal, parenteral, subcutaneous, intramuscular, intradermal, intravenous, topical, transdermal, transmucosal, inhalation, subdural injection, implantation at a lesion site, controlled release from a depot or matrix implanted or injected at a lesion site, and combinations thereof. In some embodiments, the compound of formula (II) or a pharmaceutically acceptable salt thereof is administered orally. In some embodiments, the subject is fasted when administering to the subject the therapeutically effective amount of the compound of formula (II), or a pharmaceutically acceptable salt thereof. In some embodiments, the subject is fed when administering to the subject the therapeutically effective amount of the compound of formula (II), or a pharmaceutically acceptable salt thereof. In some embodiments, the compound of formula (II), or a pharmaceutically acceptable salt thereof is administered parenterally. In some embodiments, the compound of formula (II), or a pharmaceutically acceptable salt thereof is administered once a day. In some embodiments, the compound of formula (II), or a pharmaceutically acceptable salt thereof is administered twice a day. In some embodiments, the compound of formula (II), or a pharmaceutically acceptable salt thereof is administered three times a day.

In some embodiments, wherein the subject is diagnosed with a sporadic cavernous angioma lesion. In some embodiments, the subject is diagnosed with a familial cavernous angioma lesion caused by germline loss-of-function mutations in a gene selected from the group consisting of CCM1 (KRIT1), CCM2 (malcavernin, MGC4607), or CCM3 (PDCD10). In some embodiments, the subject has multiple cavernous angioma lesions. In some embodiments, the cavernous angioma lesion has not been resected prior to administration of the compound of formula (II), or a pharmaceutically acceptable salt thereof. In some embodiments, the cavernous angioma lesion occurs in the brainstem. In some embodiments, the cavernous angioma lesion is a Stage 1 lesion. In some embodiments, the cavernous angioma lesion is a Stage 2 lesion. In some embodiments, the subject has had a symptomatic first hemorrhage prior to administration of the compound of formula (II), or a pharmaceutically acceptable salt thereof. In some embodiments, the subject has had at least one subsequent hemorrhage/rebleed following a symptomatic first hemorrhage prior to administration of the compound of formula (II), or a pharmaceutically acceptable salt thereof. In some embodiments, administering to the subject the therapeutically effective amount of the compound of formula (II), or a pharmaceutically acceptable salt thereof results in a decrease in the number of cavernous angioma lesions. In some embodiments, administering to the subject the therapeutically effective amount of the compound of formula (II), or a pharmaceutically acceptable salt thereof results in a decrease in the number of subsequent hemorrhages/rebleeds. In some embodiments, administering to the subject the therapeutically effective amount of the compound of formula (II), or a pharmaceutically acceptable salt thereof results in an improvement in blood brain barrier permeability measured by MRI imaging using the Dynamic Contrast Enhanced Quantitative Perfusion (DCEQP), Quantitative Susceptibility Mapping (QSM) or a combination thereof. In some embodiments, administering to the subject the therapeutically effective amount of the compound of formula (II), or a pharmaceutically acceptable salt thereof results in increased lesion stability measured by Quantitative Susceptibility Mapping (QSM). In some embodiments, a decrease of about 6% less in mean lesional Quantitative Susceptibility Mapping (QSM) is indicative of increased lesional stability. In some embodiments, increased lesional stability is indicative of a lack of growth or symptomatic hemorrhages/rebleeds. In some embodiments, administering to the subject the therapeutically effective amount of the compound of formula (II), or a pharmaceutically acceptable salt thereof results in an improvement in subjects' modified Rankin Scale, Mini-Mental State Examination, Euro-Quality of Life [QoL]5D, Euro-QoL, Visual Analogue Scale, Neuro-QoL, or any combination thereof. In some embodiments, administering to the subject the therapeutically effective amount of the compound of formula (II), or a pharmaceutically acceptable salt thereof results in a decrease in one or more symptoms of a symptomatic hemorrhage selected from double vision, facial droop, balance problems, difficulty swallowing, pupil and vision changes, nausea, projectile vomiting, headache, confusion, impaired consciousness and combinations thereof.

The present invention relates to a method of treating cavernous angioma lesions with symptomatic hemorrhage in a subject comprising: orally administering to the subject a therapeutically effective amount of a compound of formula (II):

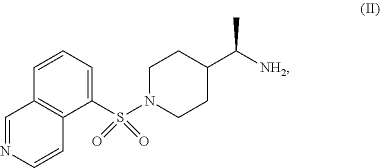 [SMILES: C[C@@H](N)C1CCN(S(=O)(=O)c2cccc3cnccc23)CC1]

or a pharmaceutically acceptable salt thereof;
wherein orally administering the therapeutically effective amount of the compound of formula (II), or a pharmaceutically acceptable salt thereof results in a Cmax of between about 3 ng/ml and about 60 ng/ml; and
wherein a Tmax is achieved of between about 0.5 hours and about 1 hour of administering the compound of formula (II), or a pharmaceutically acceptable salt thereof.

In some embodiments, the salt is an adipate salt of the compound of formula (II) or a pharmaceutically acceptable salt thereof.

In some embodiments, the therapeutically effective amount of the compound of formula (II), or a pharmaceutically acceptable salt thereof is from about 25 mg to about 250 mg.

In some embodiments, the therapeutically effective amount of the compound of formula (II), or a pharmaceutically acceptable salt thereof is an amount sufficient to result in a plasma concentration of between about 0.1 ng/ml and about 2000 ng/ml of a compound of Formula (IV):

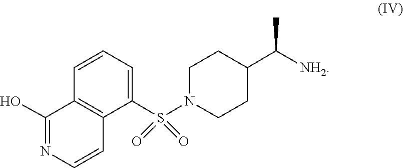 [SMILES: C[C@@H](N)C1CCN(S(=O)(=O)c2cccc3c(O)nccc23)CC1]

In some embodiments, the plasma concentration of the compound of Formula (IV) is maintained for at least 48 hours following administration of the compound of formula (II), or a pharmaceutically acceptable salt thereof. In some embodiments, the therapeutically effective amount of the compound of formula (II), or a pharmaceutically acceptable salt thereof is an amount sufficient to result in a Cmax of between about 50 ng/ml and about 1600 ng/ml of a compound of Formula (IV):

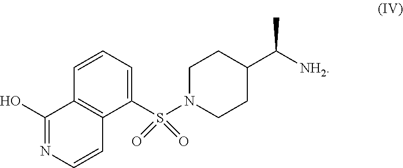 [SMILES: C[C@@H](N)C1CCN(S(=O)(=O)c2cccc3c(O)nccc23)CC1]

In some embodiments, the therapeutically effective amount of the compound of formula (II), or a pharmaceutically acceptable salt thereof is an amount sufficient to achieve a Tmax for a compound of Formula (IV):

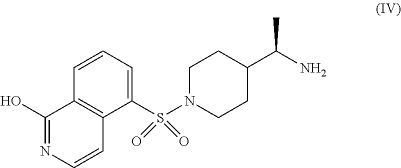 [SMILES: C[C@@H](N)C1CCN(S(=O)(=O)c2cccc3c(O)nccc23)CC1]

between about 1 hour and about 1.75 hours of administering the compound of formula (II), or a pharmaceutically acceptable salt thereof.

In some embodiments, the subject is fasted when administering to the subject the therapeutically effective amount of the compound of formula (II), or a pharmaceutically acceptable salt thereof. In some embodiments, the subject is fed when administering to the subject the therapeutically effective amount of the compound of formula (II), or a pharmaceutically acceptable salt thereof. In some embodiments, the compound of formula (II), or a pharmaceutically acceptable salt thereof is administered once a day. In some embodiments, the compound of formula (II), or a pharmaceutically acceptable salt thereof is administered twice a day. In some embodiments, the compound of formula (II), or a pharmaceutically acceptable salt thereof is administered three times a day.

In some embodiments, wherein the subject is diagnosed with a sporadic cavernous angioma lesion. In some embodiments, the subject is diagnosed with a familial cavernous angioma lesion caused by germline loss-of-function mutations in a gene selected from the group consisting of CCM1 (KRIT1), CCM2 (malcavernin, MGC4607), or CCM3 (PDCD10). In some embodiments, the subject has multiple cavernous angioma lesions. In some embodiments, the cavernous angioma lesion has not been resected prior to administration of the compound of formula (II), or a pharmaceutically acceptable salt thereof. In some embodiments, the cavernous angioma lesion occurs in the brainstem. In some embodiments, the cavernous angioma lesion is a Stage 1 lesion. In some embodiments, the cavernous angioma lesion is a Stage 2 lesion. In some embodiments, the subject has had a symptomatic first hemorrhage prior to administration of the compound of formula (II), or a pharmaceutically acceptable salt thereof. In some embodiments, the subject has had at least one subsequent hemorrhage/rebleed following a symptomatic first hemorrhage prior to oral administration of the compound of formula (II), or a pharmaceutically acceptable salt thereof. In some embodiments, orally administering to the subject the therapeutically effective amount of the compound of formula (II), or a pharmaceutically acceptable salt thereof results in a decrease in the number of cavernous angioma lesions. In some embodiments, orally administering to the subject the therapeutically effective amount of the compound of formula (II), or a pharmaceutically acceptable salt thereof results in a decrease in the number of subsequent hemorrhages/rebleeds. In some embodiments, orally administering to the subject the therapeutically effective amount of the compound of formula (II), or a pharmaceutically acceptable salt thereof results in an improvement in blood brain barrier permeability measured by MRI imaging using the Dynamic Contrast Enhanced Quantitative Perfusion (DCEQP), Quantitative Susceptibility Mapping (QSM) or a combination thereof. In some embodiments, orally administering to the subject the therapeutically effective amount of the compound of formula (II), or a pharmaceutically acceptable salt thereof results in increased lesion stability measured by Quantitative Susceptibility Mapping (QSM). In some embodiments, a decrease of about 6% less in mean lesional Quantitative Susceptibility Mapping (QSM) is indicative of increased lesional stability. In some embodiments, increased lesional stability is indicative of a lack of growth or symptomatic hemorrhages/rebleeds. In some embodiments, administering to the subject the therapeutically effective amount of the compound of formula (II), or a pharmaceutically acceptable salt thereof results in an improvement in subjects' modified Rankin Scale, Mini-Mental State Examination, Euro-Quality of Life [QoL]5D, Euro-QoL, Visual Analogue Scale, Neuro-QoL, or any combination thereof. In some embodiments, orally administering to the subject the therapeutically effective amount of the compound of formula (II), or a pharmaceutically acceptable salt thereof results in a decrease in one or more symptoms of a symptomatic hemorrhage selected from double vision, facial droop, balance problems, difficulty swallowing, pupil and vision changes, nausea, projectile vomiting, headache, confusion, impaired consciousness and combinations thereof.

The present invention also relates to a method of treating cavernous angioma lesions in a subject comprising: administering to the subject the therapeutically effective amount of the compound of formula (II):

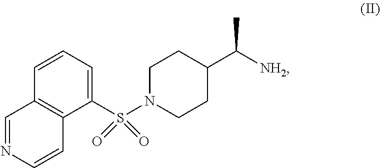 [SMILES: C[C@@H](N)C1CCN(S(=O)(=O)c2cccc3cnccc23)CC1]

or a pharmaceutically acceptable salt thereof;
wherein administering the therapeutically effective amount of the compound of formula (II), or a pharmaceutically acceptable salt thereof results in a Cmax of between about 3 ng/ml and about 60 ng/ml; and
wherein a Tmax is achieved of between about 0.5 hours and about 1 hour of administering the compound of formula (II), or a pharmaceutically acceptable salt thereof.

The present invention also relates to a method of treating cavernous angioma lesions in a subject comprising: administering to the subject the therapeutically effective amount of the compound of formula (II):

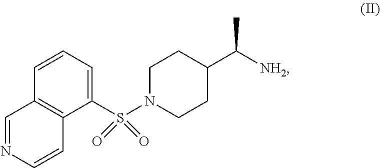 [SMILES: C[C@@H](N)C1CCN(S(=O)(=O)c2cccc3cnccc23)CC1]

or a pharmaceutically acceptable salt thereof;
wherein administering the therapeutically effective amount of the compound of formula (II), or a pharmaceutically acceptable salt thereof results in a Cmax of between 3.659 ng/ml and 58.006 ng/ml; and
wherein a Tmax is achieved of between 0.5 hours and 0.88 hours of administering the compound of formula (II), or a pharmaceutically acceptable salt thereof.

In some embodiments, the salt is an adipate salt of the compound of formula (II), or a pharmaceutically acceptable salt thereof. In some embodiments, the therapeutically effective amount of the compound of formula (II) is from about 25 mg to about 250 mg.

In some embodiments, the therapeutically effective amount of the compound of formula (II) is an amount sufficient to result in a plasma concentration of between about 0.1 ng/ml and about 2000 ng/ml of a compound of Formula (IV):

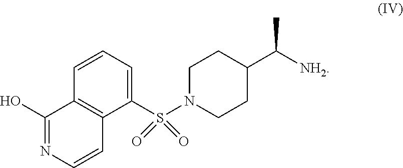 [SMILES: C[C@@H](N)C1CCN(S(=O)(=O)c2cccc3c(O)nccc23)CC1]

In some embodiments, the plasma concentration of the compound of Formula (IV) is maintained for at least 48 hours following administration of the compound of formula (II), or a pharmaceutically acceptable salt thereof.

In some embodiments, the therapeutically effective amount of the compound of formula (II), or a pharmaceutically acceptable salt thereof is an amount sufficient to result in a Cmax of between about 50 ng/ml and about 1600 ng/ml of a compound of Formula (IV):

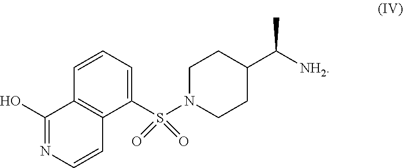 [SMILES: C[C@@H](N)C1CCN(S(=O)(=O)c2cccc3c(O)nccc23)CC1]

In some embodiments, the therapeutically effective amount of the compound of formula (II), or a pharmaceutically acceptable salt thereof is an amount sufficient to result in a Cmax of between 54.23 ng/ml and 1524.23 ng/ml of a compound of Formula (IV):

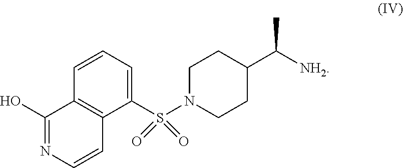 [SMILES: C[C@@H](N)C1CCN(S(=O)(=O)c2cccc3c(O)nccc23)CC1]

In some embodiments, the therapeutically effective amount of the compound of formula (II), or a pharmaceutically acceptable salt thereof is an amount sufficient to achieve a Tmax of a compound of Formula (IV):

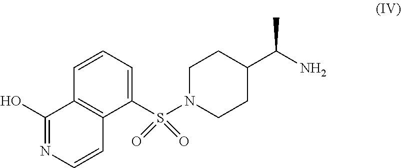 [SMILES: C[C@@H](N)C1CCN(S(=O)(=O)c2cccc3c(O)nccc23)CC1]

between about 1 hour and about 1.75 hours of administering the compound of formula (II), or a pharmaceutically acceptable salt thereof.

In some embodiments, the therapeutically effective amount of the compound of formula (II), or a pharmaceutically acceptable salt thereof is an amount sufficient to achieve a Tmax of a compound of Formula (IV):

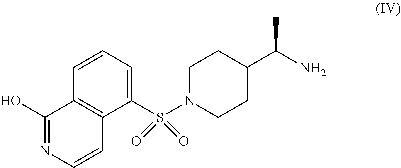 [SMILES: C[C@@H](N)C1CCN(S(=O)(=O)c2cccc3c(O)nccc23)CC1]

between 1 hour and 1.75 hours of administering the compound of formula (II), or a pharmaceutically acceptable salt thereof.

In some embodiments, the compound of formula (II), or a pharmaceutically acceptable salt thereof is administered by any suitable route of administration. In some embodiments, the compound of formula (II), or a pharmaceutically acceptable salt thereof is administered by a route selected from the group consisting of oral, rectal, topical, buccal, sub-lingual, vaginal, parenteral, subcutaneous, intramuscular, intradermal, intravenous, topical, transdermal, transmucosal, inhalation, subdural injection, implantation at a lesion site, controlled release from a depot or matrix implanted or injected at a lesion site, and combinations thereof. In some embodiments, the compound of formula (II), or a pharmaceutically acceptable salt thereof is administered orally. In some embodiments, the subject is fasted when administering to the subject the therapeutically effective amount of the compound of formula (II), or a pharmaceutically acceptable salt thereof. In some embodiments, the subject is fed when administering to the subject the therapeutically effective amount of the compound of formula (II), or a pharmaceutically acceptable salt thereof. In some embodiments, the compound of formula (II), or a pharmaceutically acceptable salt thereof is administered once a day. In some embodiments, the compound of formula (II), or a pharmaceutically acceptable salt thereof is administered twice a day. In some embodiments, the compound of formula (II), or a pharmaceutically acceptable salt thereof is administered three times a day.

In some embodiments, the subject is diagnosed with a sporadic cavernous angioma lesion. In some embodiments, the subject is diagnosed with a familial cavernous angioma lesion caused by germline loss-of-function mutations in a gene selected from the group consisting of CCM1 (KRIT1), CCM2 (malcavernin, MGC4607), CCM3 (PDCD10) or any combination thereof. In some embodiments, the subject has multiple cavernous angioma lesions. In some embodiments, the cavernous angioma lesion has not been resected prior to administration of the compound of formula (II) or a pharmaceutically acceptable salt thereof. In some embodiments, the cavernous angioma lesion occurs in the brainstem. In some embodiments, the cavernous angioma lesion is a Stage 1 lesion. In some embodiments, the cavernous angioma lesion is a Stage 2 lesion. In some embodiments, administering to the subject the therapeutically effective amount of the compound of formula (II), or a pharmaceutically acceptable salt thereof results in a decrease in the number of cavernous angioma lesions. In some embodiments, administering to the subject the therapeutically effective amount of the compound of formula (II), or a pharmaceutically acceptable salt thereof results in an improvement in blood brain barrier permeability measured by MRI imaging using the Dynamic Contrast Enhanced Quantitative Perfusion (DCEQP), Quantitative Susceptibility Mapping (QSM) or a combination thereof. In some embodiments, administering to the subject the therapeutically effective amount of the compound of formula (II), or a pharmaceutically acceptable salt thereof results in increased lesion stability measured by Quantitative Susceptibility Mapping (QSM). In some embodiments, a decrease of about 6% less in mean lesional Quantitative Susceptibility Mapping (QSM) is indicative of increased lesional stability. In some embodiments, increased lesional stability is indicative of a lack of growth, symptomatic hemorrhages/rebleeds or a combination thereof. In some embodiments, administering to the subject the therapeutically effective amount of the compound of formula (II), or a pharmaceutically acceptable salt thereof results in an improvement in subjects' modified Rankin Scale, Mini-Mental State Examination, Euro-Quality of Life [QoL]5D, Euro-QoL, Visual Analogue Scale, Neuro-QoL, or any combination thereof. In some embodiments, administering to the subject the therapeutically effective amount of the compound of formula (II), or a pharmaceutically acceptable salt thereof results in a decrease in one or more symptoms of cavernous angioma lesions. In some embodiments, the one or more symptoms of cavernous angioma lesions are selected from seizures, neurological deficits, headache, fatigue, weakness, tingling, numbness, pain, bladder issues, bowel issues, respiratory distress, and a combination thereof. In some embodiments, wherein the seizure is selected from focal onset seizures, generalized onset seizures, and a combination thereof. In some embodiments, the neurological deficits are selected from ataxia, speech and swallowing difficulties, facial paralysis, vision and auditory problems, diaphragmatic spasms, and breathing difficulties, and a combination thereof.

The present invention relates to a method of treating cavernous angioma lesions with symptomatic hemorrhage in a subject comprising: administering to the subject a therapeutically effective amount of a compound of formula (IV):

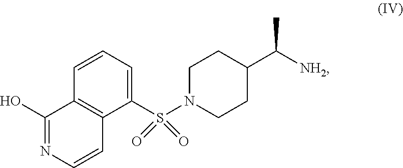 [SMILES: C[C@@H](N)C1CCN(S(=O)(=O)c2cccc3c(O)nccc23)CC1]

or a pharmaceutically acceptable salt thereof.

In some embodiments, the salt is an adipate salt of the compound of formula (IV) or a pharmaceutically acceptable salt thereof.

In some embodiments, the therapeutically effective amount of the compound of formula (IV), or a pharmaceutically acceptable salt thereof is from about 25 mg to about 250 mg.

In some embodiments, the therapeutically effective amount of the compound of formula (IV), or a pharmaceutically acceptable salt thereof is an amount sufficient to result in a plasma concentration of between about 0.1 ng/ml and about 2000 ng/ml of a compound of Formula (IV).

In some embodiments, the plasma concentration of the compound of Formula (IV) is maintained for at least 48 hours following administration. In some embodiments, the therapeutically effective amount of the compound of formula (IV), or a pharmaceutically acceptable salt thereof is an amount sufficient to result in a Cmax of between about 50 ng/ml and about 1600 ng/ml.

In some embodiments, the therapeutically effective amount of the compound of formula (IV), or a pharmaceutically acceptable salt thereof is an amount sufficient to result in a Cmax of between 54.23 ng/ml and 1524.23 ng/ml of a compound of Formula (IV).

In some embodiments, the therapeutically effective amount of the compound of formula (IV), or a pharmaceutically acceptable salt thereof is an amount sufficient to achieve a Tmax for a compound of Formula (IV) between about 1 hour and about 1.75 hours of administering the compound of formula (II), or a pharmaceutically acceptable salt thereof.

In some embodiments, the therapeutically effective amount of the compound of formula (IV), or a pharmaceutically acceptable salt thereof is an amount sufficient to achieve a Tmax for a compound of Formula (IV) between 1 hour and 1.75 hours of administering the compound of formula (II), or a pharmaceutically acceptable salt thereof.

In some embodiments, the compound of formula (IV), or a pharmaceutically acceptable salt thereof is administered by any suitable route of administration. In some embodiments, the compound of formula (IV), or a pharmaceutically acceptable salt thereof is administered by a route of administration selected from the group consisting of oral, rectal, topical, buccal, sub-lingual, vaginal, parenteral, subcutaneous, intramuscular, intradermal, intravenous, topical, transdermal, transmucosal, inhalation, subdural injection, implantation at a lesion site, controlled release from a depot or matrix implanted or injected at a lesion site, and combinations thereof. In some embodiments, the compound of formula (IV) or a pharmaceutically acceptable salt thereof is administered orally. In some embodiments, the subject is fasted when administering to the subject the therapeutically effective amount of the compound of formula (IV), or a pharmaceutically acceptable salt thereof. In some embodiments, the subject is fed when administering to the subject the therapeutically effective amount of the compound of formula (IV), or a pharmaceutically acceptable salt thereof. In some embodiments, the compound of formula (IV), or a pharmaceutically acceptable salt thereof is administered parenterally. In some embodiments, the compound of formula (IV), or a pharmaceutically acceptable salt thereof is administered once a day. In some embodiments, the compound of formula (IV), or a pharmaceutically acceptable salt thereof is administered twice a day. In some embodiments, the compound of formula (IV), or a pharmaceutically acceptable salt thereof is administered three times a day.

In some embodiments, wherein the subject is diagnosed with a sporadic cavernous angioma lesion. In some embodiments, the subject is diagnosed with a familial cavernous angioma lesion caused by germline loss-of-function mutations in a gene selected from the group consisting of CCM1 (KRIT1), CCM2 (malcavernin, MGC4607), or CCM3 (PDCD10). In some embodiments, the subject has multiple cavernous angioma lesions. In some embodiments, the cavernous angioma lesion has not been resected prior to administration of the compound of formula (IV), or a pharmaceutically acceptable salt thereof. In some embodiments, the cavernous angioma lesion occurs in the brainstem. In some embodiments, the cavernous angioma lesion is a Stage 1 lesion. In some embodiments, the cavernous angioma lesion is a Stage 2 lesion. In some embodiments, the subject has had a symptomatic first hemorrhage prior to administration of the compound of formula (IV), or a pharmaceutically acceptable salt thereof. In some embodiments, the subject has had at least one subsequent hemorrhage/rebleed following a symptomatic first hemorrhage prior to administration of the compound of formula (IV), or a pharmaceutically acceptable salt thereof. In some embodiments, administering to the subject the therapeutically effective amount of the compound of formula (IV), or a pharmaceutically acceptable salt thereof results in a decrease in the number of cavernous angioma lesions. In some embodiments, administering to the subject the therapeutically effective amount of the compound of formula (IV), or a pharmaceutically acceptable salt thereof results in a decrease in the number of subsequent hemorrhages/rebleeds. In some embodiments, administering to the subject the therapeutically effective amount of the compound of formula (IV), or a pharmaceutically acceptable salt thereof results in an improvement in blood brain barrier permeability measured by MRI imaging using the Dynamic Contrast Enhanced Quantitative Perfusion (DCEQP), Quantitative Susceptibility Mapping (QSM) or a combination thereof. In some embodiments, administering to the subject the therapeutically effective amount of the compound of formula (II), or a pharmaceutically acceptable salt thereof results in increased lesion stability measured by Quantitative Susceptibility Mapping (QSM). In some embodiments, a decrease of about 6% less in mean lesional Quantitative Susceptibility Mapping (QSM) is indicative of increased lesional stability. In some embodiments, increased lesional stability is indicative of a lack of growth or symptomatic hemorrhages/rebleeds. In some embodiments, administering to the subject the therapeutically effective amount of the compound of formula (IV), or a pharmaceutically acceptable salt thereof results in an improvement in subjects' modified Rankin Scale, Mini-Mental State Examination, Euro-Quality of Life [QoL]5D, Euro-QoL, Visual Analogue Scale, Neuro-QoL, or any combination thereof. In some embodiments, administering to the subject the therapeutically effective amount of the compound of formula (II), or a pharmaceutically acceptable salt thereof results in a decrease in one or more symptoms of a symptomatic hemorrhage selected from double vision, facial droop, balance problems, difficulty swallowing, pupil and vision changes, nausea, projectile vomiting, headache, confusion, impaired consciousness and combinations thereof.

The present invention also relates to a method of treating cavernous angioma lesions in a subject comprising: administering to the subject the therapeutically effective amount of the compound of formula (IV):

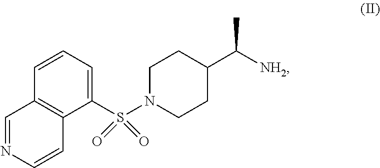 [SMILES: C[C@@H](N)C1CCN(S(=O)(=O)c2cccc3cnccc23)CC1]

or a pharmaceutically acceptable salt thereof.

In some embodiments, the salt is an adipate salt of the compound of formula (IV), or a pharmaceutically acceptable salt thereof. In some embodiments, the therapeutically effective amount of the compound of formula (IV) is from about 25 mg to about 250 mg.

In some embodiments, the therapeutically effective amount of the compound of formula (II) is an amount sufficient to result in a plasma concentration of between about 0.1 ng/ml and about 2000 ng/ml of a compound of Formula (IV).

In some embodiments, the plasma concentration of the compound of Formula (IV) is maintained for at least 48 hours following administration.

In some embodiments, the therapeutically effective amount of the compound of formula (IV), or a pharmaceutically acceptable salt thereof is an amount sufficient to result in a Cmax of between about 50 ng/ml and about 1600 ng/ml.

In some embodiments, the therapeutically effective amount of the compound of formula (IV), or a pharmaceutically acceptable salt thereof is an amount sufficient to result in a Cmax of between 54.23 ng/ml and 1524.23 ng/ml.

In some embodiments, the therapeutically effective amount of the compound of formula (IV), or a pharmaceutically acceptable salt thereof is an amount sufficient to achieve a Tmax of between about 1 hour and about 1.75 hours.

In some embodiments, the therapeutically effective amount of the compound of formula (IV), or a pharmaceutically acceptable salt thereof is an amount sufficient to achieve a Tmax of between 1 hour and 1.75 hours of administering the compound of formula (II), or a pharmaceutically acceptable salt thereof.

In some embodiments, the compound of formula (IV), or a pharmaceutically acceptable salt thereof is administered by any suitable route of administration. In some embodiments, the compound of formula (IV), or a pharmaceutically acceptable salt thereof is administered by a route selected from the group consisting of oral, rectal, topical, buccal, sub-lingual, vaginal, parenteral, subcutaneous, intramuscular, intradermal, intravenous, topical, transdermal, transmucosal, inhalation, subdural injection, implantation at a lesion site, controlled release from a depot or matrix implanted or injected at a lesion site, and combinations thereof. In some embodiments, the compound of formula (IV), or a pharmaceutically acceptable salt thereof is administered orally. In some embodiments, the subject is fasted when administering to the subject the therapeutically effective amount of the compound of formula (IV), or a pharmaceutically acceptable salt thereof. In some embodiments, the subject is fed when administering to the subject the therapeutically effective amount of the compound of formula (IV), or a pharmaceutically acceptable salt thereof. In some embodiments, the compound of formula (IV), or a pharmaceutically acceptable salt thereof is administered once a day. In some embodiments, the compound of formula (IV), or a pharmaceutically acceptable salt thereof is administered twice a day. In some embodiments, the compound of formula (IV), or a pharmaceutically acceptable salt thereof is administered three times a day.

In some embodiments, the subject is diagnosed with a sporadic cavernous angioma lesion. In some embodiments, the subject is diagnosed with a familial cavernous angioma lesion caused by germline loss-of-function mutations in a gene selected from the group consisting of CCM1 (KRIT1), CCM2 (malcavernin, MGC4607), CCM3 (PDCD10) or any combination thereof. In some embodiments, the subject has multiple cavernous angioma lesions. In some embodiments, the cavernous angioma lesion has not been resected prior to administration of the compound of formula (IV) or a pharmaceutically acceptable salt thereof. In some embodiments, the cavernous angioma lesion occurs in the brainstem. In some embodiments, the cavernous angioma lesion is a Stage 1 lesion. In some embodiments, the cavernous angioma lesion is a Stage 2 lesion. In some embodiments, administering to the subject the therapeutically effective amount of the compound of formula (IV), or a pharmaceutically acceptable salt thereof results in a decrease in the number of cavernous angioma lesions. In some embodiments, administering to the subject the therapeutically effective amount of the compound of formula (IV), or a pharmaceutically acceptable salt thereof results in an improvement in blood brain barrier permeability measured by MRI imaging using the Dynamic Contrast Enhanced Quantitative Perfusion (DCEQP), Quantitative Susceptibility Mapping (QSM) or a combination thereof. In some embodiments, administering to the subject the therapeutically effective amount of the compound of formula (IV), or a pharmaceutically acceptable salt thereof results in increased lesion stability measured by Quantitative Susceptibility Mapping (QSM). In some embodiments, a decrease of about 6% less in mean lesional Quantitative Susceptibility Mapping (QSM) is indicative of increased lesional stability. In some embodiments, increased lesional stability is indicative of a lack of growth, symptomatic hemorrhages/rebleeds or a combination thereof. In some embodiments, administering to the subject the therapeutically effective amount of the compound of formula (IV), or a pharmaceutically acceptable salt thereof results in an improvement in subjects' modified Rankin Scale, Mini-Mental State Examination, Euro-Quality of Life [QoL]5D, Euro-QoL, Visual Analogue Scale, Neuro-QoL, or any combination thereof. In some embodiments, administering to the subject the therapeutically effective amount of the compound of formula (IV), or a pharmaceutically acceptable salt thereof results in a decrease in one or more symptoms of cavernous angioma lesions. In some embodiments, the one or more symptoms of cavernous angioma lesions are selected from seizures, neurological deficits, headache, fatigue, weakness, tingling, numbness, pain, bladder issues, bowel issues, respiratory distress, and a combination thereof. In some embodiments, wherein the seizure is selected from focal onset seizures, generalized onset seizures, and a combination thereof. In some embodiments, the neurological deficits are selected from ataxia, speech and swallowing difficulties, facial paralysis, vision and auditory problems, diaphragmatic spasms, and breathing difficulties, and a combination thereof.

### Pharmaceutical Formulations

The pharmaceutical compositions useful in the therapeutic methods according to the disclosure include a therapeutically effective amount of a compound of formula (II) or a pharmaceutically acceptable salt thereof. A “therapeutically effective amount” as used herein refers to that amount which provides a therapeutic and/or prophylactic therapeutic effect for cavernous angioma. In some embodiment, a “therapeutically effective amount” as used herein refers to that amount which provides a therapeutic and/or prophylactic therapeutic effect for cavernous angioma lesion with symptomatic hemorrhage. In some embodiments, the therapeutically effective amount is from about 25 to about 150 mg. In some embodiments, the therapeutically effective amount is from about 25 to about 250 mg. In some embodiments, the therapeutically effective amount is an amount sufficient to result in a plasma concentration of between about 0.1 and about 2000 ng/ml of a compound of Formula (IV):

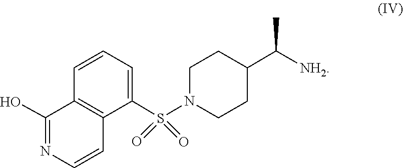 [SMILES: C[C@@H](N)C1CCN(S(=O)(=O)c2cccc3c(O)nccc23)CC1]

In some embodiments, the plasma concentration of the compound of Formula (IV) is maintained for at least 48 hours following administration of the compound of formula (II). In some embodiments, a therapeutically effective amount is from about 0.01 mg/kg to about 10 mg/kg. In some embodiments, a therapeutically effective amount is about 10 mg/kg. In some embodiments, the compound of formula (II), or a pharmaceutically acceptable salt thereof is administered by any suitable route of administration. In some embodiments, the compound of formula (II), or a pharmaceutically acceptable salt thereof is administered by a route selected from the group consisting of oral, rectal, topical, buccal, sub-lingual, vaginal, parenteral, subcutaneous, intramuscular, intradermal, intravenous, topical, transdermal, transmucosal, inhalation, subdural injection, implantation at a lesion site, controlled release from a depot or matrix implanted or injected at a lesion site, and combinations thereof. In some embodiments, the compound of formula (II) is administered orally. In some embodiments, the compound of formula (II) is administered parenterally. In some embodiments, the compound of formula (II) is administered once a day. In some embodiments, the compound of formula (II) is administered twice a day. In some embodiments, the compound of formula (II) is administered three times a day.

The pharmaceutical compositions useful in the therapeutic methods according to the disclosure include a therapeutically effective amount of a compound of formula (II) or a pharmaceutically acceptable salt thereof. A “therapeutically effective amount” as used herein refers to that amount which provides a therapeutic and/or prophylactic therapeutic effect for cavernous angioma. In some embodiment, a “therapeutically effective amount” as used herein refers to that amount which provides a therapeutic and/or prophylactic therapeutic effect for cavernous angioma lesion with symptomatic hemorrhage. In some embodiments, the therapeutically effective amount is from about 25 to about 150 mg. In some embodiments, the therapeutically effective amount is from about 25 to about 250 mg. In some embodiments, the therapeutically effective amount is an amount sufficient to result in a plasma concentration of between about 0.1 and about 2000 ng/ml of a compound of Formula (IV):

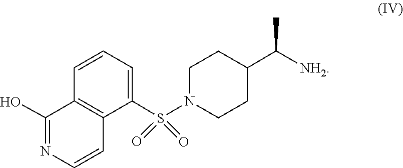 [SMILES: C[C@@H](N)C1CCN(S(=O)(=O)c2cccc3c(O)nccc23)CC1]

In some embodiments, the plasma concentration of the compound of Formula (IV) is maintained for at least 48 hours following administration of the compound of formula (II). In some embodiments, a therapeutically effective amount is from about 0.01 mg/kg to about 10 mg/kg. In some embodiments, a therapeutically effective amount is about 10 mg/kg. In some embodiments, the compound of formula (II), or a pharmaceutically acceptable salt thereof is administered by any suitable route of administration. In some embodiments, the compound of formula (II), or a pharmaceutically acceptable salt thereof is administered by a route selected from the group consisting of oral, rectal, topical, buccal, sub-lingual, vaginal, parenteral, subcutaneous, intramuscular, intradermal, intravenous, topical, transdermal, transmucosal, inhalation, subdural injection, implantation at a lesion site, controlled release from a depot or matrix implanted or injected at a lesion site, and combinations thereof. In some embodiments, the compound of formula (II) is administered orally. In some embodiments, the compound of formula (II) is administered parenterally. In some embodiments, the compound of formula (II) is administered once a day. In some embodiments, the compound of formula (II) is administered twice a day. In some embodiments, the compound of formula (II) is administered three times a day.

Such compositions can be prepared with a pharmaceutically acceptable carrier in accordance with known techniques, for example, those described in Remington. The Science And Practice of Pharmacy (9th Ed., 1995). The term “pharmaceutically acceptable carrier” is to be understood herein as referring to any substance that may, medically, be acceptably administered to a patient, together with a compound of this invention, and which does not undesirably affect the pharmacological activity thereof, a “pharmaceutically acceptable carrier” may thus be for example a pharmaceutically acceptable member(s) selected from the group comprising or consisting of diluents, preservatives, solubilizers, emulsifiers, adjuvant, tonicity modifying agents, buffers as well as any other physiologically acceptable vehicle.

Such pharmaceutically acceptable carriers include carriers known in the art such as for example, phosphate buffer solution such as 0.01 M to 0.1 M phosphate buffer and for example 0.05 M phosphate buffer or phosphate buffered saline, and 0.8% saline solution. Additionally, such pharmaceutically acceptable carriers may be aqueous or non-aqueous solutions, suspensions, and emulsions. Such solutions, suspensions, and emulsions may be aqueous. Examples of non-aqueous solvents include propylene glycol, polyethylene glycol, vegetable oils such as olive oil or soybean oil, and pharmaceutically acceptable organic esters such as ethyl oleate which are suitable for use in injectable formulations. Aqueous carriers may include water, alcoholic/aqueous solutions, emulsions or suspensions, including saline and buffered media. Parenteral vehicles may include sodium chloride solution, Ringer's dextrose, dextrose and sodium chloride, lactated Ringer's or fixed oils. Intravenous vehicles may include fluid and nutrient replenishers, electrolyte replenishers such as those based on Ringer's dextrose, and the like. Preservatives and other additives may also be present, such as, for example, antimicrobials, antioxidants, collating agents, inert gases and the like. Pharmaceutical compositions of compounds of this invention may be performed, for example, in the absence of oxygen, such as in an inert atmosphere for example nitrogen or argon. Liquids used in the preparation of formulation compositions of this invention may be sparged with an inert gas prior to use to substantially remove unwanted dissolved gases such as air and oxygen. Additionally such compositions may more particularly be liquids or lyophilized or otherwise dried formulations and include diluents of various buffer content (e.g., Tris-HCl, acetate, phosphate), pH and ionic strength; additives such as albumin or gelatin which may prevent absorption of an active compound of this invention to a surface such as glass, pharmaceutically acceptable detergents (e.g., Tween 20, Tween 80, Pluronic F68, bile acid salts); solubilizing agents (e.g., glycerol, polyethylene glycerol); anti-oxidants (e.g., ascorbic acid, sodium metabisulfite); preservatives (e.g., thimerosal, benzyl alcohol, parabens); bulking substances or tonicity modifiers (e.g., lactose, mannitol). The active agent may for example be associated with liposomes, emulsions, microemulsions, micelles, unilamellar or multilamellar vesicles, erythrocyte ghosts, or spheroplasts. The carrier element of such compositions may be chosen with an eye to influence the physical state, solubility, stability, rate of in vivo release, and rate of in vivo clearance. The compositions of this invention may comprise controlled or sustained release compositions and may comprise a compound of this invention formulated in lipophilic depots (e.g., fatty acids, waxes, oils).

Pharmaceutically acceptable acids for use in the preparation of a pharmaceutically acceptable acid addition salt of a compound of this invention include and be selected from the group consisting of acetic acid, benzenesulfonic acid, benzoic acid, bicarbonic acid, bitartaric acid, calcium dihydrogenedetic acid, camphorsulfonic acid, carbonic acid, citric acid, dodecylsulfonic acid, edetic acid, 1,2-ethanedisulfonic acid, estolic acid, ethanesulfonic acid, 2-ethylsuccinic acid, fumaric acid, glucoheptonic acid, glubionic acid, gluconic acid, glutamic acid, glycollylarsanilic acid, hexylresorcinic acid, hydrobromic acid, hydrochloric acid, hydroxynaphthoic acid, 3-hydroxynaphthoic acid, hydriodic acid, 2-hydroxyethanesulfonic acid, isethionic acid, lactic acid, lactobionic acid, laurylsulfuric acid, levulinic acid, malic acid, maleic acid, mandelic acid, methanesulfonic acid, mucic acid, naphthalenesulfonic acid, nitric acid, pamoic acid, embonic acid, pantothenic acid, phosphoric acid, polygalacturonic acid, propionic acid, salicylic acid, saccharic acid, stearic acid, subacetic acid, succinic acid, sulfamic acid, sulfuric acid, tannic acid, tartaric acid, theoclic acid, 8-chlorotheophyllinic acid, triethiodic acid, and combinations thereof.

Pharmaceutically acceptable acids for use in the preparation of a pharmaceutically acceptable salt of a compound of this invention may be selected from the group consisting of adipic, alginic, aminosalicylic, anhydromethylenecitric, arecoline, aspartic, hydrogensulfuric, camphoric, digluconic, hydrogensuccinic, glycerophosphoric, hydrofluoric, methylenebis(salicylic), napadisylic, 1,5-naphthalenedisulfonic, pectinic, persulfuric, phenylethylbarbituric, picric, propionic, thiocyanic, toluenesulfonic, and undecanoic acid, and combinations thereof.

In the preparation of a pharmaceutical composition according to the invention, a compound of formula (II) or a pharmaceutically acceptable salt thereof may include one or more physiologically acceptable salts thereof, is typically admixed with, inter alia, a pharmaceutically acceptable carrier. The carrier may be a solid or a liquid, or both, and may be formulated with the compound of formula I as a unit-dose formulation, for example, a tablet or an injectable suspension or an injectable solution, which may contain from 0.01 percent to 99 percent, or from 0.5 percent to 95 percent, by weight of the compound of formula (II) or a pharmaceutically acceptable salt thereof.

In the preparation of a pharmaceutical composition according to the invention, a compound of formula (II) or a pharmaceutically acceptable salt thereof may include one or more physiologically acceptable salts thereof, is typically admixed with, inter alia, a pharmaceutically acceptable carrier. The carrier may be a solid or a liquid, or both, and may be formulated with the compound of formula I as a unit-dose formulation, for example, a tablet or an injectable suspension or an injectable solution, which may contain from 0.01 percent to 99 percent, or from 0.5 percent to 95 percent, by weight of the compound of formula (II) or a pharmaceutically acceptable salt thereof.

The method of administration of a pharmaceutical composition of this invention may be selected from the group consisting of oral, rectal, topical, buccal, sub-lingual, vaginal, parenteral, subcutaneous, intramuscular, intradermal, intravenous, topical, transdermal, transmucosal, inhalation, subdural injection, implantation at a lesion site, controlled release from a depot or matrix implanted or injected at a lesion site, and combinations thereof. The most suitable route in any given case will depend on the nature and severity of the condition being treated. Other formulations may comprise topical and inhalation formulations.

The compound of formula (II) or a pharmaceutically acceptable salt thereof may be formulated in pharmaceutically acceptable dosage forms such as for injectable use, for oral use, for inhalation use, for transdermal use, for transmembrane use, and the like. Pharmaceutical compositions suitable for oral administration may be presented in discrete units or dosage forms, such as capsules, cachets, lozenges, tablets, pills, powders, granules, chewing gum, suspensions, solutions, and the like. Each dosage form contains a predetermined amount of a compound of this invention. Solutions and suspensions of a compound of this invention or a pharmaceutically acceptable salt thereof may be in an aqueous liquid, such as buffered with a pharmaceutically acceptable pH buffer, or in non-aqueous liquid such as DMSO, or be prepared as an oil-in-water or water-in-oil emulsion. Injectable dosage forms may be sterilized in a pharmaceutically acceptable fashion, for example by steam sterilization of an aqueous solution sealed in a vial under an inert gas atmosphere at 120° C. for about 15 to 20 minutes, or by sterile filtration of a solution through a 0.2 or smaller pore-size filter, optionally followed by a lyophilization step, or by irradiation of a composition containing a compound of the present invention by means of emissions from a radionuclide source.

The compound of formula (II) or a pharmaceutically acceptable salt thereof may be formulated in pharmaceutically acceptable dosage forms such as for injectable use, for oral use, for inhalation use, for transdermal use, for transmembrane use, and the like. Pharmaceutical compositions suitable for oral administration may be presented in discrete units or dosage forms, such as capsules, cachets, lozenges, tablets, pills, powders, granules, chewing gum, suspensions, solutions, and the like. Each dosage form contains a predetermined amount of a compound of this invention. Solutions and suspensions of a compound of this invention or a pharmaceutically acceptable salt thereof may be in an aqueous liquid, such as buffered with a pharmaceutically acceptable pH buffer, or in non-aqueous liquid such as DMSO, or be prepared as an oil-in-water or water-in-oil emulsion. Injectable dosage forms may be sterilized in a pharmaceutically acceptable fashion, for example by steam sterilization of an aqueous solution sealed in a vial under an inert gas atmosphere at 120° C. for about 15 to 20 minutes, or by sterile filtration of a solution through a 0.2 or smaller pore-size filter, optionally followed by a lyophilization step, or by irradiation of a composition containing a compound of the present invention by means of emissions from a radionuclide source.

Pharmaceutical compositions of a compound of formula (II) or a pharmaceutically acceptable salt thereof may be prepared by any suitable method of pharmacy. An exemplary method may comprise the step of bringing into association, for example by mixing, by dissolution, by suspension, by blending, by granulation, and the like, a compound of this invention or a pharmaceutically acceptable salt thereof and a pharmaceutically acceptable carrier such as a liquid, for example a liquid consisting of water, an aqueous solution of a pharmaceutically acceptable buffer, an aqueous solution of a pharmaceutically acceptable alcohol, a pharmaceutically acceptable oil such as an edible oil such as a triglyceride or mixture of triglycerides of natural sources such as an edible plant oil, an emulsion of a pharmaceutically acceptable oil in an aqueous medium comprising water, and which aqueous medium may contain one or more pharmaceutically acceptable excipients such as an excipient selected from the group consisting of a pH buffering agent, a matrix forming sugar, a pharmaceutically acceptable polymer, a pharmaceutically acceptable tonicity modifying agent, a surface modifier or surfactant useful to form micelles or to form liposomes or to form emulsions, and the like. Useful pharmaceutically acceptable excipients may be found in the Handbook of Pharmaceutical Excipients, second Edition, ed. Wade et al, 1994, which is incorporated by reference.

Pharmaceutical compositions of a compound of formula (II) or a pharmaceutically acceptable salt thereof may be prepared by any suitable method of pharmacy. An exemplary method may comprise the step of bringing into association, for example by mixing, by dissolution, by suspension, by blending, by granulation, and the like, a compound of this invention or a pharmaceutically acceptable salt thereof and a pharmaceutically acceptable carrier such as a liquid, for example a liquid consisting of water, an aqueous solution of a pharmaceutically acceptable buffer, an aqueous solution of a pharmaceutically acceptable alcohol, a pharmaceutically acceptable oil such as an edible oil such as a triglyceride or mixture of triglycerides of natural sources such as an edible plant oil, an emulsion of a pharmaceutically acceptable oil in an aqueous medium comprising water, and which aqueous medium may contain one or more pharmaceutically acceptable excipients such as an excipient selected from the group consisting of a pH buffering agent, a matrix forming sugar, a pharmaceutically acceptable polymer, a pharmaceutically acceptable tonicity modifying agent, a surface modifier or surfactant useful to form micelles or to form liposomes or to form emulsions, and the like. Useful pharmaceutically acceptable excipients may be found in the Handbook of Pharmaceutical Excipients, second Edition, ed. Wade et al, 1994, which is incorporated by reference.

The compound of formula (II) or a pharmaceutically acceptable salt thereof may also be combined in solid form with pharmaceutically acceptable excipients such as ingredients used in tablet formation such as release agents and compressing agents, silica, cellulose, methyl cellulose, hydroxypropylcellulose (HPC), polyvinylpyrolidinone (PVP), gelatin, acacia, magnesium stearate, sodium lauryl sulfate, mannitol, lactose, colorants, dyes, and formed into a dosage form such as a tablet, capsule, caplet, pill, powder, granule, and the like. Optionally, the tablet or related dosage form may be coated with a polymer coating such as an enteric and/or moisture barrier polymer coating such as may be applied by spraying, spray drying, or fluid bed drying methods.

The compound of formula (II) or a pharmaceutically acceptable salt thereof may also be combined in solid form with pharmaceutically acceptable excipients such as ingredients used in tablet formation such as release agents and compressing agents, silica, cellulose, methyl cellulose, hydroxypropylcellulose (HPC), polyvinylpyrolidinone (PVP), gelatin, acacia, magnesium stearate, sodium lauryl sulfate, mannitol, lactose, colorants, dyes, and formed into a dosage form such as a tablet, capsule, caplet, pill, powder, granule, and the like. Optionally, the tablet or related dosage form may be coated with a polymer coating such as an enteric and/or moisture barrier polymer coating such as may be applied by spraying, spray drying, or fluid bed drying methods.

The compound of formula (II) or a pharmaceutically acceptable salt thereof may be combined in an aqueous or aqueous-organic, or an organic liquid solvent together with one or more pharmaceutically acceptable excipient and then dried, for example in an inert or non-oxidizing atmosphere by spray drying, lyophilization, fluid bed drying, or evaporation to form a solid in which the compound of this invention or a pharmaceutically acceptable salt thereof is imbibed or uniformly dispersed or suspended. The pharmaceutical compositions of the invention may be prepared by admixing, such as by uniformly and intimately admixing, a compound of this invention or a pharmaceutically acceptable salt thereof, with a liquid or with a finely divided solid carrier or matrix-forming excipient or mixture of excipients, then, if necessary, shaping the resulting mixture into a dosage form suitable for the intended use such. For example, a tablet may be prepared by compressing or molding a powder or granules or granulates containing a compound of this invention or a pharmaceutically acceptable salt thereof, optionally with one or more accessory ingredients. Compressed tablets may be prepared by compressing in a tablet press a mixture of a compound of this invention or a pharmaceutically acceptable salt thereof together with one or more pharmaceutically acceptable excipient materials, which mixture may be in a free-flowing form such as a powder or granules optionally mixed with a pharmaceutically acceptable material selected from the group consisting of a binder, a lubricant, an inert diluent, a surface active agent, a dispersing agent, and combinations thereof. Molded tablets may be made by molding, in a tablet mold machine, a solid powdered mixture of a compound of this invention or a pharmaceutically acceptable salt thereof together with one or more pharmaceutically acceptable excipient, which mixture is moistened with an inert liquid binder such as water or alcohol.

The compound of formula (II) or a pharmaceutically acceptable salt thereof may be combined in an aqueous or aqueous-organic, or an organic liquid solvent together with one or more pharmaceutically acceptable excipient and then dried, for example in an inert or non-oxidizing atmosphere by spray drying, lyophilization, fluid bed drying, or evaporation to form a solid in which the compound of this invention or a pharmaceutically acceptable salt thereof is imbibed or uniformly dispersed or suspended. The pharmaceutical compositions of the invention may be prepared by admixing, such as by uniformly and intimately admixing, a compound of this invention or a pharmaceutically acceptable salt thereof, with a liquid or with a finely divided solid carrier or matrix-forming excipient or mixture of excipients, then, if necessary, shaping the resulting mixture into a dosage form suitable for the intended use such. For example, a tablet may be prepared by compressing or molding a powder or granules or granulates containing a compound of this invention or a pharmaceutically acceptable salt thereof, optionally with one or more accessory ingredients. Compressed tablets may be prepared by compressing in a tablet press a mixture of a compound of this invention or a pharmaceutically acceptable salt thereof together with one or more pharmaceutically acceptable excipient materials, which mixture may be in a free-flowing form such as a powder or granules optionally mixed with a pharmaceutically acceptable material selected from the group consisting of a binder, a lubricant, an inert diluent, a surface active agent, a dispersing agent, and combinations thereof. Molded tablets may be made by molding, in a tablet mold machine, a solid powdered mixture of a compound of this invention or a pharmaceutically acceptable salt thereof together with one or more pharmaceutically acceptable excipient, which mixture is moistened with an inert liquid binder such as water or alcohol.

A pharmaceutical composition suitable for buccal or sub-lingual administration to a patient in need of treatment by compound of this invention or a pharmaceutically acceptable salt thereof may include a lozenge such as a lozenge comprising compound of this invention or a pharmaceutically acceptable salt thereof in a flavored base such as sucrose, acacia, tragacanth, and the like; and a pastille comprising a compound of this invention or a pharmaceutically acceptable salt thereof in an inert base such as gelatin, glycerin, sucrose, acacia, and the like.

pharmaceutical compositions suitable for oral administration may be presented in discrete units or dosage forms, such as capsules, cachets, lozenges, tablets, pills, powders, granules, chewing gum, suspensions, solutions, and the like. Each dosage form contains a predetermined amount of rho kinase inhibitor compound. If in the form of a solution, the pharmaceutically acceptable carrier may be an aqueous liquid, such as buffered with a pharmaceutically acceptable pH buffer, or in non-aqueous liquid such as DMSO, or be prepared as an oil-in-water or water-in-oil emulsion.

A pharmaceutical composition of the present invention that is suitable for parenteral administration may comprise a sterile aqueous solution, and a non-aqueous solution in an organic solvent safe for injection of a compound of this invention or a pharmaceutically acceptable salt thereof of this invention. Useful injectable dosage forms containing a compound of formula (II) or a pharmaceutically acceptable salt thereof of this invention may be isotonic with the blood of the intended recipient. Tonicity of the dosage form may be adjusted and/or maintained by addition of pharmaceutically acceptable for injection water-soluble excipients such as sugars, buffer salts, and combinations thereof. These dosage forms may optionally contain antioxidants, buffers, bacteriostats, and dissolved solutes that render the formulation isotonic with the blood of the intended recipient. Aqueous and non-aqueous sterile suspensions may include pharmaceutically acceptable suspending agents and thickening agents. Formulations of this invention may be presented in unit-dose or multi-dose containers. For example, for injectable use, a formulation may be sealed in an ampoule or vial, for example, sealed in oxygen-free form such as in a vial under an inert oxygen-free gas such as nitrogen or argon or other non-reactive gas, or a mixture of non-reactive gases. In another embodiment, a dosage form of this invention may be stored in a freeze-dried or lyophilized form as an anhydrous solid or as a solid containing a small quantity of water, for example from 0.01% to about 5% by weight of the dried dosage form, which dosage form then requires the addition of a sterile liquid carrier, for example, isotonic aqueous saline solution, and optionally buffered to between about pH 5 to pH 9, or between pH 6 and pH 8, or by addition of water-for-injection immediately prior to use. Extemporaneous injection solutions and suspensions may be prepared from sterile powders, granules and tablets of the kind previously described.

A pharmaceutical composition of the present invention that is suitable for parenteral administration may comprise a sterile aqueous solution, and a non-aqueous solution in an organic solvent safe for injection of a compound of this invention or a pharmaceutically acceptable salt thereof of this invention. Useful injectable dosage forms containing a compound of formula (II) or a pharmaceutically acceptable salt thereof of this invention may be isotonic with the blood of the intended recipient. Tonicity of the dosage form may be adjusted and/or maintained by addition of pharmaceutically acceptable for injection water-soluble excipients such as sugars, buffer salts, and combinations thereof. These dosage forms may optionally contain antioxidants, buffers, bacteriostats, and dissolved solutes that render the formulation isotonic with the blood of the intended recipient. Aqueous and non-aqueous sterile suspensions may include pharmaceutically acceptable suspending agents and thickening agents. Formulations of this invention may be presented in unit-dose or multi-dose containers. For example, for injectable use, a formulation may be sealed in an ampoule or vial, for example, sealed in oxygen-free form such as in a vial under an inert oxygen-free gas such as nitrogen or argon or other non-reactive gas, or a mixture of non-reactive gases. In another embodiment, a dosage form of this invention may be stored in a freeze-dried or lyophilized form as an anhydrous solid or as a solid containing a small quantity of water, for example from 0.01% to about 5% by weight of the dried dosage form, which dosage form then requires the addition of a sterile liquid carrier, for example, isotonic aqueous saline solution, and optionally buffered to between about pH 5 to pH 9, or between pH 6 and pH 8, or by addition of water-for-injection immediately prior to use. Extemporaneous injection solutions and suspensions may be prepared from sterile powders, granules and tablets of the kind previously described.

A pharmaceutical composition of this invention containing a compound of formula (II) or a pharmaceutically acceptable salt thereof and which is suitable for rectal administration may be presented as a unit dose suppository. A suppository dosage form containing a compound of formula (II) or a pharmaceutically acceptable salt thereof may be prepared by admixing a compound of formula (II) or a pharmaceutically acceptable salt thereof with one or more conventional pharmaceutically acceptable solid carriers, for example, such as cocoa butter, to form a mixture containing a compound of this invention or a pharmaceutically acceptable salt thereof, and then shaping the resulting mixture.

A pharmaceutical composition of this invention containing a compound of formula (II) or a pharmaceutically acceptable salt thereof and which is suitable for rectal administration may be presented as a unit dose suppository. A suppository dosage form containing a compound of formula (II) or a pharmaceutically acceptable salt thereof may be prepared by admixing a compound of formula (II) or a pharmaceutically acceptable salt thereof with one or more conventional pharmaceutically acceptable solid carriers, for example, such as cocoa butter, to form a mixture containing a compound of this invention or a pharmaceutically acceptable salt thereof, and then shaping the resulting mixture.

A pharmaceutical composition of this invention suitable for transdermal administration of a compound of formula (II) or a pharmaceutically acceptable salt thereof may be presented as a discrete patch dosage form. The patch may be adapted to remain in intimate contact with the epidermis or stratum corneum of a recipient for a prolonged period of time such as from 8 hours to about 48 hours or longer. A pharmaceutical composition suitable for transdermal administration may also be delivered by an iontophoretic delivery mechanism such as by using an applied voltage difference between two portions of the dosage form, each of which is in contact with the skin of a patient.

A pharmaceutical composition of this invention suitable for transdermal administration of a compound of formula (II) or a pharmaceutically acceptable salt thereof may be presented as a discrete patch dosage form. The patch may be adapted to remain in intimate contact with the epidermis or stratum corneum of a recipient for a prolonged period of time such as from 8 hours to about 48 hours or longer. A pharmaceutical composition suitable for transdermal administration may also be delivered by an iontophoretic delivery mechanism such as by using an applied voltage difference between two portions of the dosage form, each of which is in contact with the skin of a patient.

A therapeutically effective dosage of the compound of formula (II) or a pharmaceutically acceptable salt thereof varies from patient to patient and may depend upon factors such as the age of the patient, the patient's genetics, and the diagnosed condition of the patient, and the route of delivery of the dosage form to the patient. A therapeutically effective dose and frequency of administration of a dosage form may be determined in accordance with routine pharmacological procedures known to those skilled in the art. For example, dosage amounts and frequency of administration may vary or change as a function of time and severity of the cavernous angioma lesion or the cavernous angioma lesion with symptomatic hemorrhage. For example, a dosage from about 0.1 mg/kg to 1000 mg/kg, or from about 1 mg/kg to about 100 mg/kg rho kinase inhibitor may be suitable. In some embodiments, 10 mg/kg may be suitable.

A therapeutically effective dosage of the compound of formula (II) or a pharmaceutically acceptable salt thereof varies from patient to patient, and may depend upon factors such as the age of the patient, the patient's genetics, and the diagnosed condition of the patient, and the route of delivery of the dosage form to the patient. A therapeutically effective dose and frequency of administration of a dosage form may be determined in accordance with routine pharmacological procedures known to those skilled in the art. For example, dosage amounts and frequency of administration may vary or change as a function of time and severity of the cavernous angioma lesion or the cavernous angioma lesion with symptomatic hemorrhage. For example, a dosage from about 0.1 mg/kg to 1000 mg/kg, or from about 1 mg/kg to about 100 mg/kg rho kinase inhibitor may be suitable. In some embodiments, 10 mg/kg may be suitable. In some embodiments, the therapeutically effective amount is from about 25 to about 150 mg. In some embodiments, the therapeutically effective amount is from about 25 to about 250 mg. In some embodiments, the therapeutically effective amount is an amount sufficient to result in a plasma concentration of between about 0.1 and about 2000 ng/ml of a compound of Formula (IV):

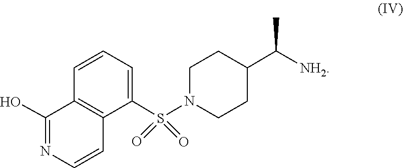 [SMILES: C[C@@H](N)C1CCN(S(=O)(=O)c2cccc3c(O)nccc23)CC1]

In some embodiments, the plasma concentration of the compound of Formula (IV) is maintained for at least 48 hours following administration of the compound of formula (II).

In some embodiments, the subject is diagnosed with a sporadic cavernous angioma lesion. In some embodiments, the subject is diagnosed with a familial cavernous angioma lesion caused by germline loss-of-function mutations in a gene selected from the group consisting of CCM1 (KRIT1), CCM2 (malcavernin, MGC4607), or CCM3 (PDCD10). In some embodiments, the subject has multiple cavernous angioma lesions. In some embodiments, the cavernous angioma lesion has not been resected prior to administration of the compound of formula (I). In some embodiments, the cavernous angioma lesion occurs in the brainstem. In some embodiments, the cavernous angioma lesion is a Stage 1 lesion. In some embodiments, the cavernous angioma lesion is a Stage 2 lesion. In some embodiments, the subject has had a symptomatic first hemorrhage prior to administration of the compound of formula (I) or a pharmaceutically acceptable salt thereof. In some embodiments, the subject has had at least one subsequent hemorrhage/rebleed following a symptomatic first hemorrhage prior to administration of the compound of formula (I) or a pharmaceutically acceptable salt thereof.

In some embodiments, the subject is diagnosed with a sporadic cavernous angioma lesion. In some embodiments, the subject is diagnosed with a familial cavernous angioma lesion caused by germline loss-of-function mutations in a gene selected from the group consisting of CCM1 (KRIT1), CCM2 (malcavernin, MGC4607), or CCM3 (PDCD10). In some embodiments, the subject has multiple cavernous angioma lesions. In some embodiments, the cavernous angioma lesion has not been resected prior to administration of the compound of formula (II). In some embodiments, the cavernous angioma lesion occurs in the brainstem. In some embodiments, the cavernous angioma lesion is a Stage 1 lesion. In some embodiments, the cavernous angioma lesion is a Stage 2 lesion. In some embodiments, the subject has had a symptomatic first hemorrhage prior to administration of the compound of formula (II) or a pharmaceutically acceptable salt thereof. In some embodiments, the subject has had at least one subsequent hemorrhage/rebleed following a symptomatic first hemorrhage prior to administration of the compound of formula (II) or a pharmaceutically acceptable salt thereof.

In some embodiments, administering to the subject the therapeutically effective amount of a compound of formula (I) or a pharmaceutically acceptable salt thereof results in a decrease in the number of cavernous angioma lesions. In some embodiments, administering to the subject the therapeutically effective amount of a compound of formula (I) or a pharmaceutically acceptable salt thereof results in a decrease in the number of subsequent hemorrhages/rebleeds.

In some embodiments, administering to the subject the therapeutically effective amount of a compound of formula (II) or a pharmaceutically acceptable salt thereof results in a decrease in the number of cavernous angioma lesions. In some embodiments, administering to the subject the therapeutically effective amount of the compound of formula (II) or a pharmaceutically acceptable salt thereof results in a decrease in the number of subsequent hemorrhages/rebleeds.

In some embodiments, administering to the subject the therapeutically effective amount of a compound of formula (I) or a pharmaceutically acceptable salt thereof results in an improvement in blood brain barrier permeability. In some embodiments, an improvement in blood brain barrier permeability is measured by MRI imaging using the Dynamic Contrast Enhanced Quantitative Perfusion (DCEQP), Quantitative Susceptibility Mapping (QSM) or a combination thereof. In some embodiments, administering to the subject the therapeutically effective amount of a compound of formula (I) or a pharmaceutically acceptable salt thereof results in increased lesion stability measured by Quantitative Susceptibility Mapping (QSM). In some embodiments, a decrease of about 6% less in mean lesional Quantitative Susceptibility Mapping (QSM) is indicative of increased lesional stability. In some embodiments, increased lesional stability is indicative of a lack of growth or symptomatic hemorrhages/rebleeds.

In some embodiments, administering to the subject the therapeutically effective amount of the compound of formula (II) or a pharmaceutically acceptable salt thereof results in an improvement in blood brain barrier permeability. In some embodiments, an improvement in blood brain barrier permeability is measured by MRI imaging using the Dynamic Contrast Enhanced Quantitative Perfusion (DCEQP), Quantitative Susceptibility Mapping (QSM) or a combination thereof. In some embodiments, administering to the subject the therapeutically effective amount of the compound of formula (II) or a pharmaceutically acceptable salt thereof results in increased lesion stability measured by Quantitative Susceptibility Mapping (QSM). In some embodiments, a decrease of about 6% less in mean lesional Quantitative Susceptibility Mapping (QSM) is indicative of increased lesional stability. In some embodiments, increased lesional stability is indicative of a lack of growth or symptomatic hemorrhages/rebleeds.

In some embodiments, administering to the subject the therapeutically effective amount of the compound of formula (I), or a pharmaceutically acceptable salt thereof results in an improvement in subjects' modified Rankin Scale, Mini-Mental State Examination, Euro-Quality of Life [QoL]5D, Euro-QoL, Visual Analogue Scale, Neuro-QoL, or any combination thereof.

In some embodiments, administering to the subject the therapeutically effective amount of the compound of formula (II), or a pharmaceutically acceptable salt thereof results in an improvement in subjects' modified Rankin Scale, Mini-Mental State Examination, Euro-Quality of Life [QoL]5D, Euro-QoL, Visual Analogue Scale, Neuro-QoL, or any combination thereof.

In some embodiments, administering to the subject the therapeutically effective amount of a compound of formula (I) or a pharmaceutically acceptable salt thereof results in a decrease in one or more symptoms of a symptomatic hemorrhage selected from double vision, facial droop, balance problems, difficulty swallowing, pupil and vision changes, nausea, projectile vomiting, headache, confusion, and impaired consciousness.

In some embodiments, administering to the subject the therapeutically effective amount of the compound of formula (II) or a pharmaceutically acceptable salt thereof results in a decrease in one or more symptoms of a symptomatic hemorrhage selected from double vision, facial droop, balance problems, difficulty swallowing, pupil and vision changes, nausea, projectile vomiting, headache, confusion, and impaired consciousness.

Some embodiments are directed to a method of treating cavernous angioma lesions in a subject comprising: administering to the subject the therapeutically effective amount of a compound of formula (I):

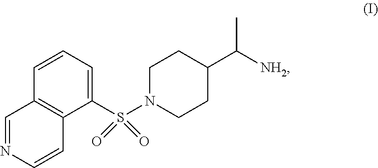 [SMILES: CC(N)C1CCN(S(=O)(=O)c2cccc3cnccc23)CC1]

or a pharmaceutically acceptable salt thereof. In some embodiments, the salt is an adipate salt of the compound of formula (I). In some embodiments, the therapeutically effective amount is from about 25 to about 150 mg. In some embodiments, the therapeutically effective amount is from about 25 to about 250 mg. In some embodiments, the therapeutically effective amount is an amount sufficient to result in a plasma concentration of between about 0.1 and about 2000 ng/ml of a compound of Formula (IV):

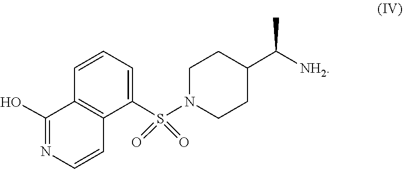 [SMILES: C[C@@H](N)C1CCN(S(=O)(=O)c2cccc3c(O)nccc23)CC1]

In some embodiments, the plasma concentration of the compound of Formula (IV) is maintained for at least 48 hours following administration of the compound of formula (I). In some embodiments, the therapeutically effective amount is from about 0.01 mg/kg to about 10 mg/kg. In some embodiments, the therapeutically effective amount is about 10 mg/kg. In some embodiments, the compound of formula (I), or a pharmaceutically acceptable salt thereof is administered by any suitable route of administration. In some embodiments, the compound of formula (I), or a pharmaceutically acceptable salt thereof is administered by a route selected from the group consisting of oral, rectal, topical, buccal, sub-lingual, vaginal, parenteral, subcutaneous, intramuscular, intradermal, intravenous, topical, transdermal, transmucosal, inhalation, subdural injection, implantation at a lesion site, controlled release from a depot or matrix implanted or injected at a lesion site, and combinations thereof. In some embodiments, the compound of formula (I) is administered orally. In some embodiments, the compound of formula (I) is administered parenterally. In some embodiments, the compound of formula (I) is administered once a day. In some embodiments, the compound of formula (I) is administered twice a day. In some embodiments, the compound of formula (I) is administered three times a day.

Some embodiments are directed to a method of treating cavernous angioma lesions in a subject comprising: administering to the subject the therapeutically effective amount of a compound of formula (II):

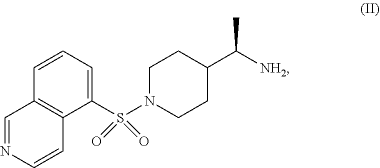 [SMILES: C[C@@H](N)C1CCN(S(=O)(=O)c2cccc3cnccc23)CC1]

or a pharmaceutically acceptable salt thereof. In some embodiments, the salt is an adipate salt of the compound of formula (II). In some embodiments, the therapeutically effective amount is from about 25 to about 150 mg. In some embodiments, the therapeutically effective amount is from about 25 to about 250 mg. In some embodiments, the therapeutically effective amount is an amount sufficient to result in a plasma concentration of between about 0.1 and about 2000 ng/ml of a compound of Formula (IV):

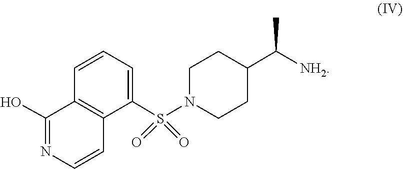 [SMILES: C[C@@H](N)C1CCN(S(=O)(=O)c2cccc3c(O)nccc23)CC1]

In some embodiments, the plasma concentration of the compound of Formula (IV) is maintained for at least 48 hours following administration of the compound of formula (II). In some embodiments, the therapeutically effective amount is from about 0.01 mg/kg to about 10 mg/kg. In some embodiments, the therapeutically effective amount is about 10 mg/kg. In some embodiments, the compound of formula (II), or a pharmaceutically acceptable salt thereof is administered by any suitable route of administration. In some embodiments, the compound of formula (II), or a pharmaceutically acceptable salt thereof is administered by a route selected from the group consisting of oral, rectal, topical, buccal, sub-lingual, vaginal, parenteral, subcutaneous, intramuscular, intradermal, intravenous, topical, transdermal, transmucosal, inhalation, subdural injection, implantation at a lesion site, controlled release from a depot or matrix implanted or injected at a lesion site, and combinations thereof. In some embodiments, the compound of formula (II) is administered orally. In some embodiments, the compound of formula (II) is administered parenterally. In some embodiments, the compound of formula (II) is administered once a day. In some embodiments, the compound of formula (II) is administered twice a day. In some embodiments, the compound of formula (II) is administered three times a day.

In some embodiment, the cavernous angioma lesion is cavernous angioma lesions with symptomatic hemorrhage. In some embodiments, the subject is diagnosed with a sporadic cavernous angioma lesion. In some embodiments, the subject is diagnosed with a familial cavernous angioma lesion caused by germline loss-of-function mutations in a gene selected from the group consisting of CCM1 (KRIT1), CCM2 (malcavernin, MGC4607), or CCM3 (PDCD10). In some embodiments, the subject has multiple cavernous angioma lesions.

In some embodiments, the cavernous angioma lesion has not been resected prior to administration of the compound of formula (I) or a pharmaceutically acceptable salt thereof. In some embodiments, the cavernous angioma lesion occurs in the brainstem. In some embodiments, the cavernous angioma lesion is a Stage 1 lesion. In some embodiments, the cavernous angioma lesion is a Stage 2 lesion.

In some embodiments, the cavernous angioma lesion has not been resected prior to administration of the compound of formula (II) or a pharmaceutically acceptable salt thereof. In some embodiments, the cavernous angioma lesion occurs in the brainstem. In some embodiments, the cavernous angioma lesion is a Stage 1 lesion. In some embodiments, the cavernous angioma lesion is a Stage 2 lesion.

In some embodiments, administering to the subject the therapeutically effective amount of a compound of formula (I) results in a decrease in the number of cavernous angioma lesions. In some embodiments, administering to the subject the therapeutically effective amount of a compound of formula (I) or a pharmaceutically acceptable salt thereof results in an improvement in blood brain barrier permeability measured by MRI imaging using the Dynamic Contrast Enhanced Quantitative Perfusion (DCEQP), Quantitative Susceptibility Mapping (QSM) or a combination thereof. In some embodiments, administering to the subject the therapeutically effective amount of a compound of formula (I) or a pharmaceutically acceptable salt thereof results in increased lesion stability measured by Quantitative Susceptibility Mapping (QSM). In some embodiments, a decrease of about 6% less in mean lesional Quantitative Susceptibility Mapping (QSM) is indicative of increased lesional stability. In some embodiments, increased lesional stability is indicative of a lack of growth or symptomatic hemorrhages/rebleeds.

In some embodiments, administering to the subject the therapeutically effective amount of the compound of formula (II) results in a decrease in the number of cavernous angioma lesions. In some embodiments, administering to the subject the therapeutically effective amount of the compound of formula (II) or a pharmaceutically acceptable salt thereof results in an improvement in blood brain barrier permeability measured by MRI imaging using the Dynamic Contrast Enhanced Quantitative Perfusion (DCEQP), Quantitative Susceptibility Mapping (QSM) or a combination thereof. In some embodiments, administering to the subject the therapeutically effective amount of the compound of formula (II) or a pharmaceutically acceptable salt thereof results in increased lesion stability measured by Quantitative Susceptibility Mapping (QSM). In some embodiments, a decrease of about 6% less in mean lesional Quantitative Susceptibility Mapping (QSM) is indicative of increased lesional stability. In some embodiments, increased lesional stability is indicative of a lack of growth or symptomatic hemorrhages/rebleeds.

In some embodiments, administering to the subject the therapeutically effective amount of a compound of formula (I) or a pharmaceutically acceptable salt thereof results in an improvement in subjects' modified Rankin Scale, Mini-Mental State Examination, Euro-Quality of Life [QoL]5D, Euro-QoL, Visual Analogue Scale, Neuro-QoL, or any combination thereof.

In some embodiments, administering to the subject the therapeutically effective amount of the compound of formula (II) or a pharmaceutically acceptable salt thereof results in an improvement in subjects' modified Rankin Scale, Mini-Mental State Examination, Euro-Quality of Life [QoL]5D, Euro-QoL, Visual Analogue Scale, Neuro-QoL, or any combination thereof.

In some embodiments, administering to the subject the therapeutically effective amount of a compound of formula (I) or a pharmaceutically acceptable salt thereof results in a decrease in one or more symptoms of cavernous angioma lesions. In some embodiments, the one or more symptoms of cavernous angioma lesions are selected from seizures, neurological deficits, headache, fatigue, weakness, tingling, numbness, pain, bladder issues, bowel issues, respiratory distress, or a combination thereof. In some embodiments, the seizure is selected from focal onset seizures, generalized onset seizures, or a combination thereof. In some embodiments, the neurological deficits are selected from ataxia, speech and swallowing difficulties, facial paralysis, vision and auditory problems, diaphragmatic spasms, and breathing difficulties, or a combination thereof.

In some embodiments, administering to the subject the therapeutically effective amount of the compound of formula (II) or a pharmaceutically acceptable salt thereof results in a decrease in one or more symptoms of cavernous angioma lesions. In some embodiments, the one or more symptoms of cavernous angioma lesions are selected from seizures, neurological deficits, headache, fatigue, weakness, tingling, numbness, pain, bladder issues, bowel issues, respiratory distress, or a combination thereof. In some embodiments, the seizure is selected from focal onset seizures, generalized onset seizures, or a combination thereof. In some embodiments, the neurological deficits are selected from ataxia, speech and swallowing difficulties, facial paralysis, vision and auditory problems, diaphragmatic spasms, and breathing difficulties, or a combination thereof.

Some embodiments are directed to a method of treating psychiatric disorders (including but not limited to, schizophrenia), severe vascular disease, stroke, vascular conditions, sickle cell disease, cerebral amyloid angiopathy, pulmonary hypertension, chronic graft versus host disease, fibrosis, conditions relating to vascular tone, ophthalmological diseases (including but not limited to glaucoma, diabetic retinopathy, primary corneal endothelial disease), in a subject comprising: administering to the subject the therapeutically effective amount of a compound of formula (I):

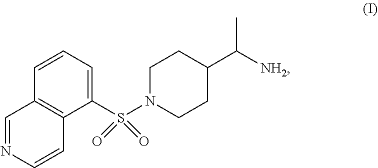 [SMILES: CC(N)C1CCN(S(=O)(=O)c2cccc3cnccc23)CC1]

or a pharmaceutically acceptable salt thereof. In some embodiments, the salt is an adipate salt of the compound of formula (I). In some embodiments, the therapeutically effective amount is from about 25 to about 150 mg. In some embodiments, the therapeutically effective amount is from about 25 to about 250 mg. In some embodiments, the therapeutically effective amount is an amount sufficient to result in a plasma concentration of between about 0.1 and about 2000 ng/ml of a compound of Formula (IV):

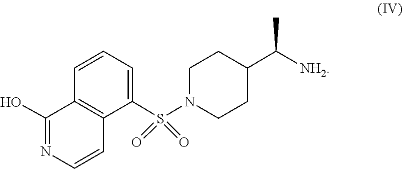 [SMILES: C[C@@H](N)C1CCN(S(=O)(=O)c2cccc3c(O)nccc23)CC1]

In some embodiments, the plasma concentration of the compound of Formula (IV) is maintained for at least 48 hours following administration of the compound of formula (I). In some embodiments, the therapeutically effective amount is from about 0.01 mg/kg to about 10 mg/kg. In some embodiments, the therapeutically effective amount is about 10 mg/kg. In some embodiments, the compound of formula (I), or a pharmaceutically acceptable salt thereof is administered by any suitable route of administration. In some embodiments, the compound of formula (I), or a pharmaceutically acceptable salt thereof is administered by a route selected from the group consisting of oral, rectal, topical, buccal, sub-lingual, vaginal, parenteral, subcutaneous, intramuscular, intradermal, intravenous, topical, transdermal, transmucosal, inhalation, subdural injection, implantation at a lesion site, controlled release from a depot or matrix implanted or injected at a lesion site, and combinations thereof. In some embodiments, the compound of formula (I) is administered orally. In some embodiments, the compound of formula (I) is administered parenterally. In some embodiments, the compound of formula (I) is administered once a day. In some embodiments, the compound of formula (I) is administered twice a day. In some embodiments, the compound of formula (I) is administered three times a day.

Some embodiments are directed to a method of treating a psychiatric disorders (including but not limited to, schizophrenia), severe vascular disease, stroke, vascular conditions, sickle cell disease, cerebral amyloid angiopathy, pulmonary hypertension, chronic graft versus host disease, fibrosis, conditions relating to vascular tone, ophthalmological diseases (including but not limited to glaucoma, diabetic retinopathy, primary corneal endothelial disease) in a subject comprising: administering to the subject the therapeutically effective amount of a compound of formula (II):

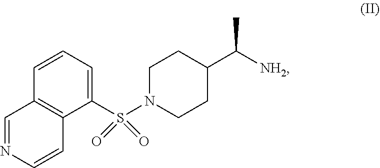 [SMILES: C[C@@H](N)C1CCN(S(=O)(=O)c2cccc3cnccc23)CC1]

or a pharmaceutically acceptable salt thereof. In some embodiments, the salt is an adipate salt of the compound of formula (II). In some embodiments, the therapeutically effective amount is from about 25 to about 150 mg. In some embodiments, the therapeutically effective amount is from about 25 to about 250 mg. In some embodiments, the therapeutically effective amount is an amount sufficient to result in a plasma concentration of between about 0.1 and about 2000 ng/ml of a compound of Formula (IV):

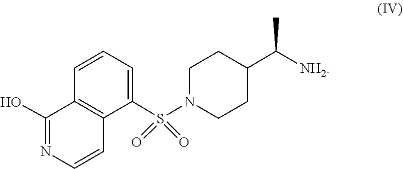 [SMILES: C[C@@H](N)C1CCN(S(=O)(=O)c2cccc3c(O)nccc23)CC1]

In some embodiments, the plasma concentration of the compound of Formula (IV) is maintained for at least 48 hours following administration of the compound of formula (II). In some embodiments, the therapeutically effective amount is from about 0.01 mg/kg to about 10 mg/kg. In some embodiments, the therapeutically effective amount is about 10 mg/kg. In some embodiments, the compound of formula (II), or a pharmaceutically acceptable salt thereof is administered by any suitable route of administration. In some embodiments, the compound of formula (II), or a pharmaceutically acceptable salt thereof is administered by a route selected from the group consisting of oral, rectal, topical, buccal, sub-lingual, vaginal, parenteral, subcutaneous, intramuscular, intradermal, intravenous, topical, transdermal, transmucosal, inhalation, subdural injection, implantation at a lesion site, controlled release from a depot or matrix implanted or injected at a lesion site, and combinations thereof. In some embodiments, the compound of formula (II) is administered orally. In some embodiments, the compound of formula (II) is administered parenterally. In some embodiments, the compound of formula (II) is administered once a day. In some embodiments, the compound of formula (II) is administered twice a day. In some embodiments, the compound of formula (II) is administered three times a day.

Some embodiments are directed to a method of treating psychiatric disorders (including but not limited to, schizophrenia), severe vascular disease, stroke, vascular conditions, sickle cell disease, cerebral amyloid angiopathy, pulmonary hypertension, chronic graft versus host disease, fibrosis, conditions relating to vascular tone, ophthalmological diseases (including but not limited to glaucoma, diabetic retinopathy, primary corneal endothelial disease), in a subject comprising: administering to the subject the therapeutically effective amount of a compound of formula (IV):

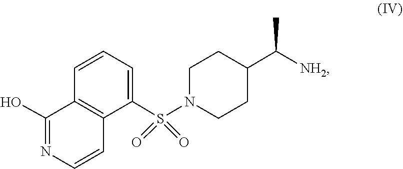 [SMILES: C[C@@H](N)C1CCN(S(=O)(=O)c2cccc3c(O)nccc23)CC1]

or a pharmaceutically acceptable salt thereof.

Some embodiments are directed to a method of treating cavernous angioma lesions with symptomatic hemorrhage in a subject comprising: administering to the subject a therapeutically effective amount of a compound of formula (II):

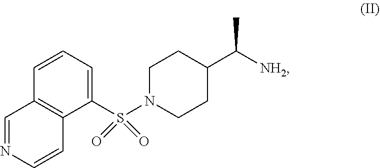 [SMILES: C[C@@H](N)C1CCN(S(=O)(=O)c2cccc3cnccc23)CC1]

or a pharmaceutically acceptable salt thereof;
wherein administering the therapeutically effective amount of the compound of formula (II), or a pharmaceutically acceptable salt thereof results in a Cmax of between about 3 ng/ml and about 60 ng/ml; and
wherein a Tmax is achieved of between about 0.5 hours and about 1 hour of administering the compound of formula (II), or a pharmaceutically acceptable salt thereof.

Some embodiments are directed to a method of treating cavernous angioma lesions with symptomatic hemorrhage in a subject comprising: administering to the subject a therapeutically effective amount of a compound of formula (II):

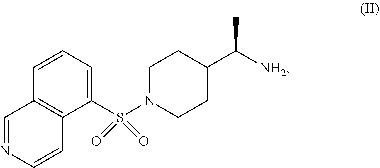 [SMILES: C[C@@H](N)C1CCN(S(=O)(=O)c2cccc3cnccc23)CC1]

or a pharmaceutically acceptable salt thereof;
wherein administering the therapeutically effective amount of the compound of formula (II), or a pharmaceutically acceptable salt thereof results in a Cmax of between 3.659 ng/ml and 58.006 ng/ml; and
wherein a Tmax is achieved of between 0.5 hours and 0.88 hours of administering the compound of formula (II), or a pharmaceutically acceptable salt thereof.

In some embodiments, the salt is an adipate salt of the compound of formula (II) or a pharmaceutically acceptable salt thereof.

In some embodiments, the therapeutically effective amount of the compound of formula (II), or a pharmaceutically acceptable salt thereof is from about 25 mg to about 250 mg.

In some embodiments, the therapeutically effective amount of the compound of formula (II), or a pharmaceutically acceptable salt thereof is an amount sufficient to result in a plasma concentration of between about 0.1 ng/ml and about 2000 ng/ml of a compound of Formula (IV):

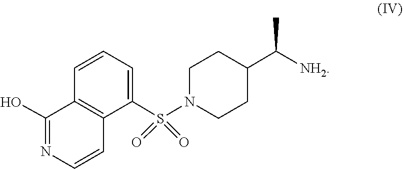 [SMILES: C[C@@H](N)C1CCN(S(=O)(=O)c2cccc3c(O)nccc23)CC1]

In some embodiments, the plasma concentration of the compound of Formula (IV) is maintained for at least 48 hours following administration of the compound of formula (II), or a pharmaceutically acceptable salt thereof. In some embodiments, the therapeutically effective amount of the compound of formula (II), or a pharmaceutically acceptable salt thereof is an amount sufficient to result in a Cmax of between about 50 ng/ml and about 1600 ng/ml of a compound of Formula (IV):

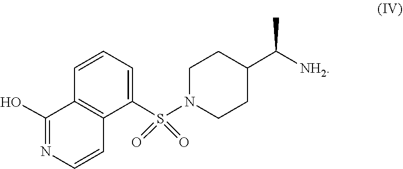 [SMILES: C[C@@H](N)C1CCN(S(=O)(=O)c2cccc3c(O)nccc23)CC1]

In some embodiments, the therapeutically effective amount of the compound of formula (II), or a pharmaceutically acceptable salt thereof is an amount sufficient to result in a Cmax of between 54.23 ng/ml and 1524.23 ng/ml of a compound of Formula (IV):

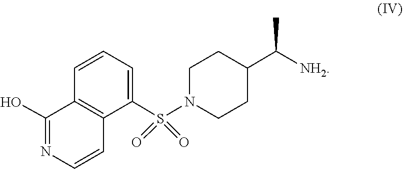 [SMILES: C[C@@H](N)C1CCN(S(=O)(=O)c2cccc3c(O)nccc23)CC1]

In some embodiments, the therapeutically effective amount of the compound of formula (II), or a pharmaceutically acceptable salt thereof is an amount sufficient to achieve a Tmax for a compound of Formula (IV):

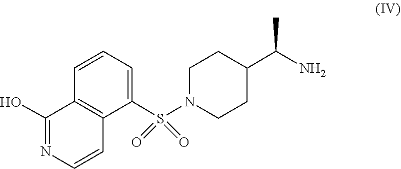 [SMILES: C[C@@H](N)C1CCN(S(=O)(=O)c2cccc3c(O)nccc23)CC1]

between about 1 hour and about 1.75 hours of administering the compound of formula (II), or a pharmaceutically acceptable salt thereof.

In some embodiments, the therapeutically effective amount of the compound of formula (II), or a pharmaceutically acceptable salt thereof is an amount sufficient to achieve a Tmax for a compound of Formula (IV):

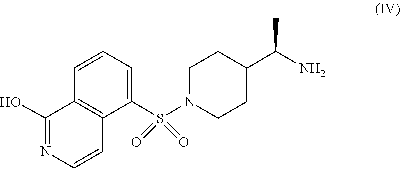 [SMILES: C[C@@H](N)C1CCN(S(=O)(=O)c2cccc3c(O)nccc23)CC1]

between 1 hour and 1.75 hours of administering the compound of formula (II), or a pharmaceutically acceptable salt thereof.

In some embodiments, the compound of formula (II), or a pharmaceutically acceptable salt thereof is administered by any suitable route of administration. In some embodiments, the compound of formula (II), or a pharmaceutically acceptable salt thereof is administered by a route selected from the group consisting of oral, rectal, topical, buccal, sub-lingual, vaginal, parenteral, subcutaneous, intramuscular, intradermal, intravenous, topical, transdermal, transmucosal, inhalation, subdural injection, implantation at a lesion site, controlled release from a depot or matrix implanted or injected at a lesion site, and combinations thereof. In some embodiments, the compound of formula (II) or a pharmaceutically acceptable salt thereof is administered orally. In some embodiments, the subject is fasted when administering to the subject the therapeutically effective amount of the compound of formula (II), or a pharmaceutically acceptable salt thereof. In some embodiments, the subject is fed when administering to the subject the therapeutically effective amount of the compound of formula (II), or a pharmaceutically acceptable salt thereof. In some embodiments, the compound of formula (II), or a pharmaceutically acceptable salt thereof is administered parenterally. In some embodiments, the compound of formula (II), or a pharmaceutically acceptable salt thereof is administered once a day. In some embodiments, the compound of formula (II), or a pharmaceutically acceptable salt thereof is administered twice a day. In some embodiments, the compound of formula (II), or a pharmaceutically acceptable salt thereof is administered three times a day.

In some embodiments, wherein the subject is diagnosed with a sporadic cavernous angioma lesion. In some embodiments, the subject is diagnosed with a familial cavernous angioma lesion caused by germline loss-of-function mutations in a gene selected from the group consisting of CCM1 (KRIT1), CCM2 (malcavernin, MGC4607), or CCM3 (PDCD10). In some embodiments, the subject has multiple cavernous angioma lesions. In some embodiments, the cavernous angioma lesion has not been resected prior to administration of the compound of formula (II), or a pharmaceutically acceptable salt thereof. In some embodiments, the cavernous angioma lesion occurs in the brainstem. In some embodiments, the cavernous angioma lesion is a Stage 1 lesion. In some embodiments, the cavernous angioma lesion is a Stage 2 lesion. In some embodiments, the subject has had a symptomatic first hemorrhage prior to administration of the compound of formula (II), or a pharmaceutically acceptable salt thereof. In some embodiments, the subject has had at least one subsequent hemorrhage/rebleed following a symptomatic first hemorrhage prior to administration of the compound of formula (II), or a pharmaceutically acceptable salt thereof. In some embodiments, administering to the subject the therapeutically effective amount of the compound of formula (II), or a pharmaceutically acceptable salt thereof results in a decrease in the number of cavernous angioma lesions. In some embodiments, administering to the subject the therapeutically effective amount of the compound of formula (II), or a pharmaceutically acceptable salt thereof results in a decrease in the number of subsequent hemorrhages/rebleeds. In some embodiments, administering to the subject the therapeutically effective amount of the compound of formula (II), or a pharmaceutically acceptable salt thereof results in an improvement in blood brain barrier permeability measured by MRI imaging using the Dynamic Contrast Enhanced Quantitative Perfusion (DCEQP), Quantitative Susceptibility Mapping (QSM) or a combination thereof. In some embodiments, administering to the subject the therapeutically effective amount of the compound of formula (II), or a pharmaceutically acceptable salt thereof results in increased lesion stability measured by Quantitative Susceptibility Mapping (QSM). In some embodiments, a decrease of about 6% less in mean lesional Quantitative Susceptibility Mapping (QSM) is indicative of increased lesional stability. In some embodiments, increased lesional stability is indicative of a lack of growth or symptomatic hemorrhages/rebleeds. In some embodiments, administering to the subject the therapeutically effective amount of the compound of formula (II), or a pharmaceutically acceptable salt thereof results in an improvement in subjects' modified Rankin Scale, Mini-Mental State Examination, Euro-Quality of Life [QoL]5D, Euro-QoL, Visual Analogue Scale, Neuro-QoL, or any combination thereof. In some embodiments, administering to the subject the therapeutically effective amount of the compound of formula (II), or a pharmaceutically acceptable salt thereof results in a decrease in one or more symptoms of a symptomatic hemorrhage selected from double vision, facial droop, balance problems, difficulty swallowing, pupil and vision changes, nausea, projectile vomiting, headache, confusion, impaired consciousness and combinations thereof.

The present invention also relates to a method of treating cavernous angioma lesions in a subject comprising: administering to the subject the therapeutically effective amount of the compound of formula (II):

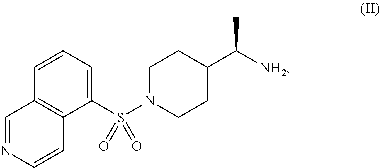 [SMILES: C[C@@H](N)C1CCN(S(=O)(=O)c2cccc3cnccc23)CC1]

or a pharmaceutically acceptable salt thereof;
wherein administering the therapeutically effective amount of the compound of formula (II), or a pharmaceutically acceptable salt thereof results in a Cmax of between about 3 ng/ml and about 60 ng/ml; and
wherein a Tmax is achieved of between about 0.5 hours and about 1 hour of administering the compound of formula (II), or a pharmaceutically acceptable salt thereof.

The present invention also relates to a method of treating cavernous angioma lesions in a subject comprising: administering to the subject the therapeutically effective amount of the compound of formula (II):

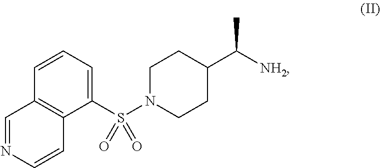 [SMILES: C[C@@H](N)C1CCN(S(=O)(=O)c2cccc3cnccc23)CC1]

or a pharmaceutically acceptable salt thereof;
wherein administering the therapeutically effective amount of the compound of formula (II), or a pharmaceutically acceptable salt thereof results in a Cmax of between 3.659 ng/ml and 58.006 ng/ml; and
wherein a Tmax is achieved of between 0.5 hours and 0.88 hours of administering the compound of formula (II), or a pharmaceutically acceptable salt thereof.

In some embodiments, the salt is an adipate salt of the compound of formula (II), or a pharmaceutically acceptable salt thereof. In some embodiments, the therapeutically effective amount of the compound of formula (II) is from about 25 mg to about 250 mg.

In some embodiments, the therapeutically effective amount of the compound of formula (II) is an amount sufficient to result in a plasma concentration of between about 0.1 ng/ml and about 2000 ng/ml of a compound of Formula (IV):

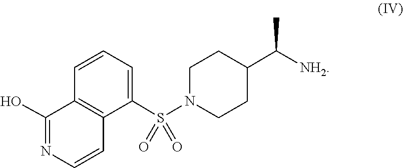 [SMILES: C[C@@H](N)C1CCN(S(=O)(=O)c2cccc3c(O)nccc23)CC1]

In some embodiments, the plasma concentration of the compound of Formula (IV) is maintained for at least 48 hours following administration of the compound of formula (II), or a pharmaceutically acceptable salt thereof.

In some embodiments, the therapeutically effective amount of the compound of formula (II), or a pharmaceutically acceptable salt thereof is an amount sufficient to result in a Cmax of between about 50 ng/ml and about 1600 ng/ml of a compound of Formula (IV):

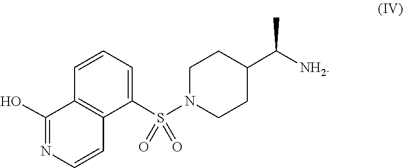 [SMILES: C[C@@H](N)C1CCN(S(=O)(=O)c2cccc3c(O)nccc23)CC1]

In some embodiments, the therapeutically effective amount of the compound of formula (II), or a pharmaceutically acceptable salt thereof is an amount sufficient to achieve a Tmax of a compound of Formula (IV):

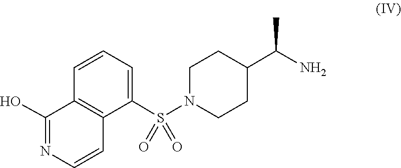 [SMILES: C[C@@H](N)C1CCN(S(=O)(=O)c2cccc3c(O)nccc23)CC1]

between about 1 hour and about 1.75 hours of administering the compound of formula (II), or a pharmaceutically acceptable salt thereof.

In some embodiments, the therapeutically effective amount of the compound of formula (II), or a pharmaceutically acceptable salt thereof is an amount sufficient to result in a Cmax of between 54.23 ng/ml and 1524.23 ng/ml of a compound of Formula (IV):

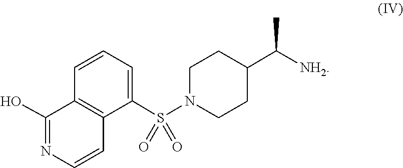 [SMILES: C[C@@H](N)C1CCN(S(=O)(=O)c2cccc3c(O)nccc23)CC1]

In some embodiments, the therapeutically effective amount of the compound of formula (II), or a pharmaceutically acceptable salt thereof is an amount sufficient to achieve a Tmax of a compound of Formula (IV):

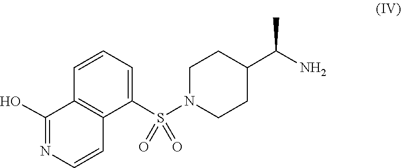 [SMILES: C[C@@H](N)C1CCN(S(=O)(=O)c2cccc3c(O)nccc23)CC1]

between 1 hour and about 1.75 hours of administering the compound of formula (II), or a pharmaceutically acceptable salt thereof.

In some embodiments, the compound of formula (II), or a pharmaceutically acceptable salt thereof is administered by any suitable route of administration. In some embodiments, the compound of formula (II), or a pharmaceutically acceptable salt thereof is administered by a route selected from the group consisting of oral, rectal, topical, buccal, sub-lingual, vaginal, parenteral, subcutaneous, intramuscular, intradermal, intravenous, topical, transdermal, transmucosal, inhalation, subdural injection, implantation at a lesion site, controlled release from a depot or matrix implanted or injected at a lesion site, and combinations thereof. In some embodiments, the compound of formula (II), or a pharmaceutically acceptable salt thereof is administered orally. In some embodiments, the subject is fasted when administering to the subject the therapeutically effective amount of the compound of formula (II), or a pharmaceutically acceptable salt thereof. In some embodiments, the subject is fed when administering to the subject the therapeutically effective amount of the compound of formula (II), or a pharmaceutically acceptable salt thereof. In some embodiments, the compound of formula (II), or a pharmaceutically acceptable salt thereof is administered once a day. In some embodiments, the compound of formula (II), or a pharmaceutically acceptable salt thereof is administered twice a day. In some embodiments, the compound of formula (II), or a pharmaceutically acceptable salt thereof is administered three times a day.

In some embodiments, the subject is diagnosed with a sporadic cavernous angioma lesion. In some embodiments, the subject is diagnosed with a familial cavernous angioma lesion caused by germline loss-of-function mutations in a gene selected from the group consisting of CCM1 (KRIT1), CCM2 (malcavernin, MGC4607), CCM3 (PDCD10) or any combination thereof. In some embodiments, the subject has multiple cavernous angioma lesions. In some embodiments, the cavernous angioma lesion has not been resected prior to administration of the compound of formula (II) or a pharmaceutically acceptable salt thereof. In some embodiments, the cavernous angioma lesion occurs in the brainstem. In some embodiments, the cavernous angioma lesion is a Stage 1 lesion. In some embodiments, the cavernous angioma lesion is a Stage 2 lesion. In some embodiments, administering to the subject the therapeutically effective amount of the compound of formula (II), or a pharmaceutically acceptable salt thereof results in a decrease in the number of cavernous angioma lesions. In some embodiments, administering to the subject the therapeutically effective amount of the compound of formula (II), or a pharmaceutically acceptable salt thereof results in an improvement in blood brain barrier permeability measured by MRI imaging using the Dynamic Contrast Enhanced Quantitative Perfusion (DCEQP), Quantitative Susceptibility Mapping (QSM) or a combination thereof. In some embodiments, administering to the subject the therapeutically effective amount of the compound of formula (II), or a pharmaceutically acceptable salt thereof results in increased lesion stability measured by Quantitative Susceptibility Mapping (QSM). In some embodiments, a decrease of about 6% less in mean lesional Quantitative Susceptibility Mapping (QSM) is indicative of increased lesional stability. In some embodiments, increased lesional stability is indicative of a lack of growth, symptomatic hemorrhages/rebleeds or a combination thereof. In some embodiments, administering to the subject the therapeutically effective amount of the compound of formula (II), or a pharmaceutically acceptable salt thereof results in an improvement in subjects' modified Rankin Scale, Mini-Mental State Examination, Euro-Quality of Life [QoL]5D, Euro-QoL, Visual Analogue Scale, Neuro-QoL, or any combination thereof. In some embodiments, administering to the subject the therapeutically effective amount of the compound of formula (II), or a pharmaceutically acceptable salt thereof results in a decrease in one or more symptoms of cavernous angioma lesions. In some embodiments, the one or more symptoms of cavernous angioma lesions are selected from seizures, neurological deficits, headache, fatigue, weakness, tingling, numbness, pain, bladder issues, bowel issues, respiratory distress, and a combination thereof. In some embodiments, wherein the seizure is selected from focal onset seizures, generalized onset seizures, and a combination thereof. In some embodiments, the neurological deficits are selected from ataxia, speech and swallowing difficulties, facial paralysis, vision and auditory problems, diaphragmatic spasms, and breathing difficulties, and a combination thereof.

### Example 1—Preclinical Studies in Transgenic Mouse Models

CCM3 and CCM1 transgenic mouse models replicate CCM disease in humans. Lesions increase in number over time, lesions grow and become leaky; iron deposition and new lesions are formed. CCM3 is most aggressive disease genotype and has earlier onset, prevalent in pediatric population.

NRL-1049 was administered to mice in drinking water after weaning. Three doses plus placebo were tested. For the CCM3 model 90 days of NRL-1049 treatment and for the CCM1 model 120 days of NRL-1049 treatment.

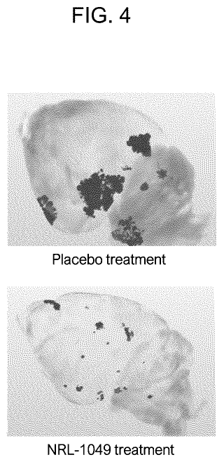

Fewer lesions were observed following NRL-1049 treatment versus placebo in the Ccm3+/−Trp53−/− mouse model. See FIG. 4 FIG. 4 shows the effect of the compound of formula (II) (NRL-1049) versus placebo in the Ccm3+/−Trp53−/− mouse model (red areas are lesions).

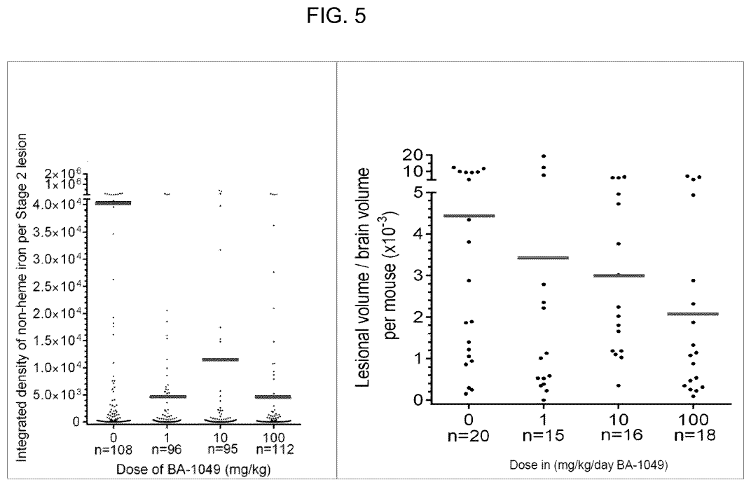

There appears to be a dose dependent lesion reduction in the Ccm3+/−Trp53−/− mouse model. FIG. 5 shows the integrated density of non-heme iron per stage 2 lesion and lesional volume/brain volume per mouse with various doses of compound of formula (II) (NRL-1049) (1, 10, and 100 mg/kg) versus placebo. Significant decreases in overall lesion volume at higher dose concentrations was observed and there were significant decreases in non-heme iron at all dose concentrations tested. Lesions are large compared to CCM1 mice and fewer large stage 2 lesions at all doses were observed.

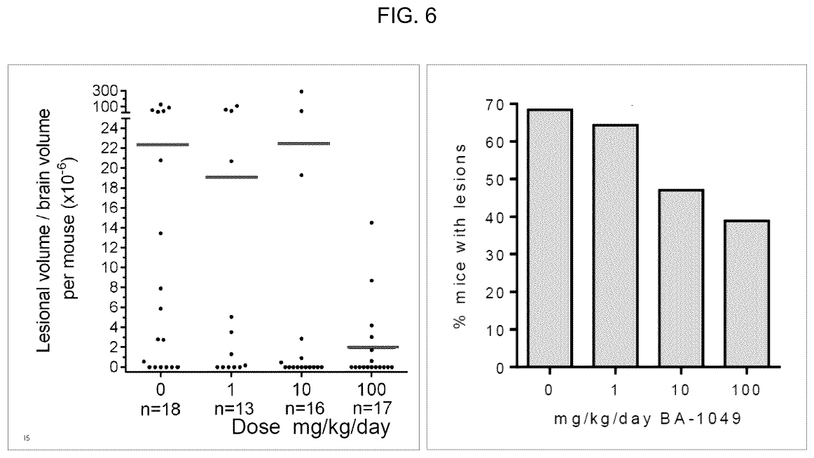

The also appears to be a dose dependent lesion reduction in the Ccm1+/−Msh2+/− model. FIG. 6 shows lesional volume/brain volume per mouse and the percentage of mice with lesions with various doses of compound of formula (II) (NRL-1049) (1, 10, and 100 mg/kg/day) versus placebo. Few large lesions were observed at all doses with a significant decrease in overall lesion volume at high doses and a dose dependent decrease in the number of mice that have lesions.

In a cryo-injury model, dosing the compound of formula (II) (NRL-1049) followed by NRL-1049 IP resulted in no seizures in the NRL-1049 arm, compared to >60% in vehicle arm. There was also reduced edema in the NRL-1049 arm indicative of decreased endothelial leakage.

In a transient middle cerebral artery occlusion model, decreased phosphorylation of p-Cofilin, a marker of ROCK 2 activation in vivo and a reduction in intra-infarct hemorrhage—indicative of decreased endothelial leakage was observed with dosing the compound of formula (II) (NRL-1049).

### Example 2—Nonclinical Pharmacokinetics and Metabolism

After a single oral dose in mice, rats, and monkeys, NRL-1049 is rapidly absorbed with time to maximum plasma concentration (Tmax) ranging from 0.25 to 2.0 h and rapidly cleared with elimination phase half-life (t½) ranging from 0.57 to 6.8 h. There is a large first pass metabolism of NRL-1049 to NRL-2017 (1-hydroxy NRL-1049), which is a pharmacologically active, potent ROCK 2 inhibitor. The Tmax and t½ of NRL-2017 ranges from 0.5 to 4 h and 0.58 to 5.7 h, respectively. Exposure to NRL-1049 and NRL-2017 generally increased in a greater than dose-proportional manner. Exposure to NRL-2017 was similar or greater (up to 5-fold) than that to NRL-1049 in rats when dosed once a day (QD). In monkeys, exposure to NRL-2017 was also greater (up to 33-fold) than that to NRL-1049. When rats were dosed twice a day (BID) for 14 days, there was a decrease in formation of NRL-2017 with exposure to NRL-2017 lower than that of NRL-1049 (ratio as low as 0.06). When incubated with mouse, monkey, and human hepatocytes, NRL-1049 had moderate to high clearance. NRL-1049 was not metabolized by dog hepatocytes. In rat hepatocytes, the clearance rate was moderate with formation of two metabolites, NRL-2017 and an N-oxide metabolite (the latter is not a ROCK 2 inhibitor). Both NRL-1049 and NRL-2017 were distributed to brain and vascular tissue (aorta, inferior vena cava) and were excreted in urine. Plasma protein binding in mouse, rat, dog, monkey, minipig and human plasma was low to moderate with % free drug ranging from 54.1% to 70.6%. NRL-1049 bound to human serum albumin and human alpha1-acid glycoprotein. Metabolism was primarily mediated by cytochrome P450 (CYP) isozyme CYP2D6. NRL-1049 inhibited CYP2C8, CYP2D6 and CYP3A4/5 with half-maximal inhibitory concentration (IC50) of 160, 0.18 and 23/110 μM, respectively. There was no metabolite or time-dependent inhibition. Induction of CYP1A2 (3-fold) and CYP3A4 (half-maximal effective concentration [EC50], 41.5 μM; maximal effective concentration [Emax], 2.19-fold) was noted in at least one hepatocyte culture. Reduced expression of CYP2B2 to 0.5-fold was noted at concentrations as low as 3.2 μM. NRL-1049 inhibited organic anion transporter (OAT) OAT1, organic cation transporters (OCT) OCT1 and OCT2, and multidrug and toxin extrusion (MATE) MATE1 (IC50, 8.54 to 66.2 μM). There was also inhibition of P-glycoprotein (P-gp) (only CACO-2 cells), organic anion transporting polypeptides (OATP) OATP1B1, OATP1B3 and OAT3 (IC50, >100 μM), but no effect on P-gp (MDCK2 cells), breast cancer resistance protein (BCRP), bile salt export pump (BSEP), or MATE2-K. NRL-1049 is a substrate for P-gp, not BCRP. In the absence of clinical data, using the murine peak plasma concentration (Cmax) for efficacy indicates that the inhibition of CYP isozymes, induction of CYP isozymes and inhibition of transporters does not occur at clinically relevant concentrations except for inhibition of CYP2D6. This conclusion will be revisited when human pharmacokinetic (PK) data becomes available.

### Example 3—Human Single Ascending Dose Study

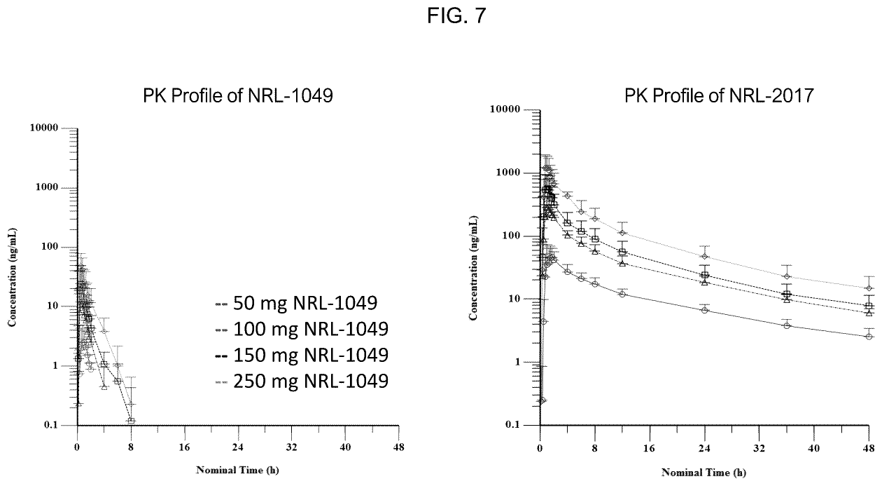

In a first-in human single ascending dose (25, 50, 150 and 250 mg) study, the pharmacokinetic profile of NRL-1049 and its metabolite NRL-2017 was established. FIG. 7 shows the PK profile for NRL-1049 and its metabolite NRL-2017 following administration of a single dose of NRL-1049 of 25, 50, 150 or 250 mg.

### Example 4—A First-in-Human, Randomized, Dose-Escalation, Double-Blind, Placebo-Controlled Single Ascending Dose Study to Establish Safety, Tolerability, and Pharmacokinetic Parameters of NRL-1049 in Healthy Volunteers

### Study Rationale

Cerebral cavernous malformations (CCMs, also known as “cavernous angioma” or “cavernoma”) are lesions characterized by abnormally enlarged capillary cavities most commonly found in the cerebral cortex, brainstem, and spinal cord. In the CCM lesions, vascular walls are abnormally thin and dilated and ultrastructural analysis reveals ruptures in the endothelium indicating physical breakage between cells or loss of junctional integrity endothelium, resulting in an increase in vascular permeability.1

Cerebral cavernous malformations can present as solitary or multiple lesions and, depending on size and location, can be “clinically silent” or present with clinical symptoms. The severity and overall clinical impact are highly dependent on the location and number of lesions in each individual. However, the existence of even one lesion may predispose a patient to seizures, stroke, other focal neurological deficits, and death. Patients with CCM lesions typically manifest with headache, epilepsy, or hemorrhagic stroke at a mean age of approximately 30 years, with a wide range of age of onset.1

Patients afflicted with CCMs have limited treatment options, which include symptom management and in some well selected cases surgical intervention, contingent upon the location, number of lesions, and the size of the lesion. The majority of CCMs are conservatively managed by evaluating the emergence of new symptoms and/or radiological changes on serial monitoring. Ideally, a drug that disrupts the pathophysiology and progression of CCM and reduces de novo lesion formation and growth over time would benefit these patients by reducing the risk of morbidity and mortality.1

The formation of the lesions with resultant increase in potential hemorrhage has been directly linked to a disruption in the delicate Rho signaling equilibrium within endothelial cells with CCM loss of function. Rho kinase (ROCK) 2 overactivation in the capillary endothelium, which leads to excessive stress fiber formation, destabilization of endothelial cell-cell contacts, increased vascular permeability, and reprogramming of endothelial cells into senescence associated secretory phenotype. As a result, the vasculature shows reduced endothelial integrity, abnormal vascular growth, aberrant angiogenesis and capillary malformation, and higher permeability and risk of bleeding.1

For this reason, a pharmacological inhibitor of ROCK 2 as a target, the most common ROCK enzyme in the brain vasculature, is a perfect candidate for treating CCM. There are no approved treatments for CCM in the US, however an NRL-1049 analog (Fasudil) has been approved in Japan for improvement of cerebral vasospasm and associated cerebral ischemic symptoms following surgery for subarachnoid hemorrhage by inhibiting ROCK. Based on such attributes, further supported by preclinical data, it is proposed that NRL-1049, a ROCK 2-selective inhibitor, could fulfill this unmet medical need both for the acute treatment as well as the chronic prevention of lesion development and progression, leading to secondary cerebral bleeding.1

Nonclinical studies have been performed to investigate the pharmacology, pharmacokinetics (PK), and toxicology of NRL-1049 and its major metabolite NRL-2017. Details of these studies are provided in the Investigator's Brochure.¹

NRL-1049 had not been previously administered to humans. The present study was planned as the first-in-human study in the development program of NRL-1049 to assess the safety, tolerability, and pharmacokinetics (PK) of single ascending doses of NRL-1049 in healthy human volunteers, as well as to explore the food effect on the PK of NRL 1049.

TABLE 1
Study Objectives

| | |
|---|---|
| OBJECTIVES | ENDPOINTS |

Primary

| | |
|---|---|
| To determine the maximum tolerated dose | Treatment-emergent (AEs) and serious AEs |
| of a single oral dose of NRL-1049 | Clinical laboratory tests, physical examination |
| To collect data on clinical safety and | findings, vital signs, 12-lead |
| tolerability | electrocardiograms, and cardiac telemetry |

Secondary

| | |
|---|---|
| To assess the pharmacokinetics (PK) of | Plasma PK parameters for NRL-1049 and its |
| NRL-1049 and its active metabolite, | active metabolite, NRL-2017, in the fasting |
| NRL-2017, after single oral doses of | state and for NRL-1049 only in the fed state: |
| NRL-1049 | Maximum observed concentration (Cₘₐₓ) |
| To evaluate the effect of food on the PK of | Area under the concentration-time curve |
| NRL-1049 | (AUC) from time 0 to the time of the last |
| | quantifiable concentration (AUC_{0-T}) |
| | AUC from time 0 extrapolated to infinity |
| | (AUC_{inf}) |
| | AUC from time 0 to the median time that |
| | Cₘₐₓ is observed (Tₘₐₓ) (AUC_{0-Tmax}) |
| | Time to maximum drug concentration |
| | (Tₘₐₓ) |
| | Terminal phase elimination rate constant (λ_{z}) |
| | Terminal phase elimination half-life (T_{half}) |
| | Apparent clearance (CL/F) |
| | Apparent volume of distribution during the |
| | terminal phase (V_{Z}/F) |

Exploratory

| | |
|---|---|
| To assess the effects of a single oral dose | Measurement of P-cofilin levels in blood |
| of NRL-1049 on the levels of P-cofilin in | |
| blood, as a potential biomarker of rho | |
| kinase activity | |

### Overall Study Design and Plan

This was a first-in-human, single center, randomized, double-blind, placebo-controlled, single ascending dose (SAD) study, including a food-effect assessment, in healthy male and female subjects. Up to 44 subjects were included in the study.

Subjects who provided informed consent underwent screening evaluations within 28 days prior to study drug administration. Screening data was reviewed to determine subject eligibility. Subjects who met all inclusion criteria and none of the exclusion criteria were included in the study.

The study consisted of 4 sequential cohorts of 8 subjects each (6 active and 2 placebo), randomly assigned to receive a single oral dose of NRL-1049 or matching-placebo under fasting state on Day 1 (See Table 2). In each cohort, a sentinel group of 2 subjects (1 active and 1 placebo) were dosed at least 24 hours prior to enrolling the remaining subjects of the cohort, contingent on the safety profile observed during sentinel dosing, as defined by medical judgment.

TABLE 2
Schedule of Single Ascending Dose Cohorts

| | | N | | Drug |
|---|---|---|---|---|
| Cohort | | (active:placebo) | NRL-1049 Dose Level | administration |
| 1 | Sentinel | 1:1 | 25 mg: 1 × 25 mg capsule | Single oral |
| | Cohort | 5:1 | | NRL-1049 or |
| 2 | Sentinel | 1:1 | 75 mg: 1 × 75 mg capsule | placebo |
| | Cohort | 5:1 | | administration |
| 3 | Sentinel | 1:1 | 150 mg: 2 × 75 mg capsules | under fasting |
| | Cohort | 5:1 | | state |
| 4 | Sentinel | 1:1 | 250 mg: (3 × 75) + (1 × 25) mg | |
| | Cohort | 5:1 | capsules | |

Subjects were admitted to the clinical research unit (CRU) on Day −1. On Day 1, following an overnight fast, subjects received a single oral dose of their assigned study treatment. Safety, PK, and exploratory biomarker assessments were performed prior to dosing and at assigned time points during study confinement. Subjects were permitted to leave the CRU following the completion of the 48-hour PK postdose sampling and safety assessments. A safety follow-up phone call was performed on Day 8 (±1 day).

For the SAD cohorts (excluding food-effect Cohort 3), the duration of subject participation, including Screening, was approximately 37 days.

Each dose cohort was separated by at least 7 days to review safety by the Data Safety Monitoring Board (DSMB) prior to a decision to proceed with the next higher dose cohort or stop the study.

Furthermore, a food-effect assessment was integrated into Cohort 3, in which a single oral 150 mg dose of NRL-1049 or placebo was administered to 12 subjects (10 active and 2 placebo) under fed state in Period 2 (refer to schedule of food-effect Cohort 3 in Table 3). After completion of Period 1 and a washout period of at least 14 days, Cohort 3 subjects repeated the screening procedures to ensure they still met the inclusion and exclusion criteria prior to readmission to the CRU for Period 2. Subjects dosed in Period 1 and who returned for Period 2 (a maximum of 6 active and 2 placebo) were administered the same single oral dose in Period 2 under fed state. In addition, at least 4 new subjects were enrolled in Period 2 only, in order to dose a total of 12 subjects (10 active and 2 placebo), in a 1:1 ratio of male to female. If a subject dosed in Period 1 was unable to return for Period 2, a new subject was enrolled to ensure dosing a total of 12 subjects in Period 2. Newly enrolled subjects only received the study drug in the fed state, and as such were not required to undergo Period 1 activities. There was no sentinel approach in Period 2.

TABLE 3
Schedule of Food-Effect Cohort 3

| | | N | | | N | |
|---|---|---|---|---|---|---|
| Cohort | | (active:placebo) | Period 1 | Washout | (active:placebo) | Period 2^{a} |
| Cohort 3 | Sentinel | 1:1 | Single oral 150 mg | 14 days | 10:2 | Single oral 150 mg |
| | Cohort | 5:1 | NRL-1049 or placebo | | | NRL-1049 or placebo |
| | | | administration under | | | administration under |
| | | | fasting state | | | fed state |

The safety, PK, and exploratory biomarker assessments were performed prior to dosing and at assigned time points during study confinement. For the food-effect cohort, exploratory biomarker samples were collected in both periods. Subjects were discharged from the CRU following the completion of the 48-hour PK postdose sampling and safety assessments. A safety follow-up phone call was performed on Day 23 (±1 day).

For the food-effect Cohort 3 (Periods 1 and 2), the duration of subject participation, including Screening, was approximately 51 days. For new subjects enrolled only for Period 2 of Cohort 3, the duration of subject participation, including Screening, was approximately 37 days.

### Discussion of Study Design

This first-in-human SAD study was designed to determine the MTD, collect data on the safety and tolerability of NRL-1049, assess the PK of NRL-1049 and its active metabolite (NRL-2017), evaluate the effect of food on the PK of NRL-1049, and explore the effect of NRL-1049 on P cofilin in blood, as a potential biomarker of ROCK activity, following oral administration of NRL 1049 in healthy male and female volunteers.

### Selection of Study Population

Subjects who met all the inclusion criteria and none of the exclusion criteria at the screening visit were eligible for participation in this study. Continued eligibility was assessed upon admission to the clinical site, prior to the first study drug administration.

Inclusion Criteria are as follows: Provision of signed and dated ICF prior to Screening; Stated willingness to comply with all study procedures and to follow site and local infectious disease safety protocol, and availability for the duration of the study; Healthy adult male or female; Females of nonchildbearing potential were postmenopausal (defined as a minimum of 12 consecutive months of spontaneous amenorrhea confirmed by a serum follicle stimulating hormone [FSH] level>40 IU/L) or surgically sterile (hysterectomy, bilateral oophorectomy, or bilateral tubal ligation); Females of child-bearing potential agreed to use a highly effective method of contraception from 3 months before study drug administration through 90 days after the final dose of study drug; Males who were sexually active and whose partners are females of childbearing potential agreed to use condoms from the first administration of study drug until at least 90 days after administration of the last dose of study drug and ensured that their female partner used an acceptable form of contraception; Aged at least 18 years but not older than 55 years; Body mass index (BMI) within 18.0 kg/m2 to 32.0 kg/m2 (inclusive); Body weight between 76 and 111 kg (inclusive); Had no clinically significant (CS) diseases captured in the medical history or evidence of CS findings on the physical examination (including vital signs) and/or electrocardiogram (ECG), as determined by an Investigator; Subject were able to communicate effectively with the study personnel

Exclusion Criteria are as follows: Female who was lactating; Female who was pregnant according to the pregnancy test at Screening or prior to the first study drug administration; History of significant hypersensitivity to ROCK kinase inhibitors or any related products (including excipients of the formulations) as well as severe hypersensitivity reactions (like angioedema) to any drugs; Presence or history of significant gastrointestinal (GI), liver or kidney disease, or surgery (with the exception of appendectomy) that may have affected drug bioavailability; History of significant cardiovascular, pulmonary, hematologic, neurological, psychiatric, endocrine, immunologic, or dermatologic disease; History of surgery or major trauma within 30 days of Screening, or surgery planned during the study; Presence of CS ECG abnormalities at the screening visit, as defined by medical judgment; Subjects who (for whatever reason) had been on an abnormal diet (such as one that severely restricts specific basic food groups [eg, ketogenic diet], limits calories [eg, fast], and/or required the use of daily supplements as a substitute for the foods typically eaten at mealtimes) during the 30 days preceding Screening; Smoking and the use of tobacco products, tetrahydrocannabinol (THC), or cannabidiol (CBD) in the 6 months prior to the first dose of study drug; History of alcohol abuse (consumption of >10 standard drinks/week), illicit drug use, physical dependence to any opioid, THC, CBD, or any history of drug abuse or addiction within 12 months of Screening; Any CS illness in the 28 days prior to the first study drug administration; Use of any prescription drugs (with the exception of hormonal contraceptives or hormone replacement therapy) in the 28 days prior to the first study drug administration, that in the opinion of an Investigator would put into question the status of the participant as healthy; Use of over-the-counter products, including vitamins and supplements, in the 7 days prior to the first study drug administration; Use of St. John's wort in the 28 days prior to the first study drug administration; Treatment with any known enzyme-altering drugs such as barbiturates, phenothiazines, cimetidine, carbamazepine, etc., within 30 days prior to the first dose of study drug or during the study; Positive test result for alcohol, cotinine, and/or drugs of abuse at Screening or prior to the first drug administration; Positive screening results to HIV Ag/Ab combo, hepatitis B surface antigen, or hepatitis C virus tests; Any other CS abnormalities in laboratory test results at Screening that would have, in the opinion of an Investigator, increased the subject's risk of participation, jeopardized complete participation in the study, or compromised interpretation of study data; Inclusion in a previous group for this clinical study; Intake of another investigational drug or participated in any clinical study within 30 days or 5 half-lives of the investigational drug's PK or biological activity (if known), whichever was longer, prior to the first study drug administration of this study; Any other condition or prior therapy that, in the Investigator's opinion, would have made the subject unsuitable for the study, or unable or unwilling to comply with the study procedures; Donation of plasma in the 7 days prior to the first study drug administration; Donation of 1 unit of blood to American Red Cross or equivalent organization or donation of over 500 mL of blood in the 56 days prior to the first study drug administration; Involved in the planning or conduct of this study; Unwilling or unlikely to comply with the requirements of the study.

### Selection and Timing of Dose for Each Subject

Study drugs (NRL-1049 or placebo) were administered in the morning. The date and time of each dose was recorded. For each subject, all scheduled postdose activities and assessments were performed relative to the time of study drug administration. An oral dose of the assigned formulation was administered to subjects with approximately 240 mL of water at ambient temperature. The capsule(s) were swallowed whole and not chewed or broken. Food and fluid intake other than water were controlled for each confinement period and all subjects. In Cohorts 1, 2, 3 (Period 1), and 4, water was permitted as needed except from 1 hour predose until 1 hour after dosing. In Period 2 of Cohort 3, water was permitted as needed throughout the confinement period. For cohorts under fasting state, subjects were required to fast (abstain from food) for at least 10 hours prior to dosing and for at least 4 hours following dosing. In Period 2 of the food-effect cohort only, following an overnight fast of at least 10 hours, subjects received a standardized high-fat, high-calorie meal 30 minutes prior to study drug administration. An example meal would consist of 2 eggs fried in butter, 2 strips of bacon, 2 slices of toast with butter, 4 ounces of hash brown potatoes, and 8 ounces of whole milk. Substitutions in this test meal could have been made provided that the meal delivered a similar amount of calories from protein, carbohydrate, and fat and had comparable meal volume and texture. Subjects had to eat the total content of this meal in 30 minutes or less. For all cohorts, a standardized lunch was served at least 4 hours after dosing. A supper, light snack, and other meals were served at appropriate times thereafter, but not until after 9 hours postdose.

### Plasma Samples for Pharmacokinetic Analysis

Blood samples for PK measurements were collected prior to and at 0.08 (5 minutes), 0.17 (10 minutes), 0.33 (20 minutes), 0.5 (30 minutes), 0.75 (45 minutes), 1 (60 minutes), 1.25, 1.5, 1.75, 2, 4, 6, 8, 12, 24, 36, and 48 hours following drug administration. For the food effect cohort, the same blood sampling schedule was used for Period 2 (fed state). Blood samples were collected by direct venipuncture or, if judged necessary by site staff, blood samples were collected from an indwelling cannula (stylet catheter that requires no flushing). The time of PK blood sample collection was calculated relative to the time of treatment administration.

### Sample Analysis

The direct measurements of this study were the plasma concentrations of NRL-1049 and NRL 2017, which were assayed using a validated method. All details concerning the performance of the bioanalytical method used during the study can be found in the Bioanalytical Report presented separately.

### Pharmacokinetic Parameters

A PK analysis was performed by non-compartmental analysis and the plasma PK parameters presented in Table 4 were calculated for NRL-1049 and NRL-2017.

TABLE 4
Pharmacokinetic Parameters of
NRL-1049 and NRL-2017 in Plasma

| Parameter | Definition |
|---|---|
| Cₘₐₓ | Maximum observed concentration |
| Tₘₐₓ | Time to maximum drug concentration |
| AUC_{0-T} | Area under the concentration-time curve (AUC) from time |
| | 0 to the time of the last quantifiable concentration |
| AUC_{inf} | AUC from time 0 extrapolated to infinity |
| AUC_{0-Tmax} | AUC from time 0 to the median time that Cₘₐₓ is observed |
| | (Tₘₐₓ) |
| λ_{Z} | Terminal elimination rate constant |
| T_{half} | Terminal elimination half-life |
| CL/F | Apparent clearance |
| V_{Z}/F | Apparent volume of distribution during the terminal phase |

The following PK parameters were used for PK calculation and
presented in the PK listings only

| | |
|---|---|
| λ_{Z Upper} | Upper limit on time for values included in the calculation |
| | of λ_{Z} |
| λ_{Z Lower} | Lower limit on time for values included in the calculation |
| | of λ_{Z} |
| R² | Goodness of fit for the terminal phase |
| Number of | Number of data points in computing λ_{Z} |
| Points | |

### Exploratory Biomarker Assessments

Blood samples for evaluation of P-cofilin levels were collected prior to dosing and at 2, 12, and 24 hours postdose. For the food-effect cohort, samples were collected in both periods. These samples were processed, stored, and shipped according to the sample processing instructions supplied by the testing facility where samples were analyzed.

TABLE 5
Demographic Characteristics (Safety Population) - Part 1 (SAD Assessment) (Abbreviations: BMI =
body mass index; Max = maximum; Min = minimum; N = number of subjects who
received the specified treatment; n = number of subjects/events in a category; SD = standard deviation).

| | | Cohort 1 | Cohort 2 | Cohort 3 | Cohort 4 | |
|---|---|---|---|---|---|---|
| | | NRL-1049 | NRL-1049 | NRL-1049 | NRL-1049 | Placebo |
| | | 25 mg | 75 mg | 150 mg | 250 mg | Combined |
| | | (N = 6) | (N = 6) | (N = 6) | (N = 6) | (N = 8) |
| Age (years) | Mean (SD) | 39.7 | 45.8 | 36.0 | 36.0 | 33.4 |
| | | (11.52) | (7.22) | (7.67) | (5.37) | (4.81) |
| | Median | 40.5 | 46.0 | 37.5 | 34.0 | 33.0 |
| | Min, Max | 24, 51 | 37, 54 | 26, 46 | 30, 44 | 27, 42 |
| Sex [n (%)] | Male | 5 | 5 | 5 | 3 | 6 |
| | | (83.3) | (83.3) | (83.3) | (50.0) | (75.0) |
| | Female | 1 | 1 | 1 | 3 | 2 |
| | | (16.7) | (16.7) | (16.7) | (50.0) | (25.0) |
| Ethnicity [n | Hispanic/Latino | 1 | 2 | 4 | 1 | 3 |
| (%)] | | (16.7) | (33.3) | (66.7) | (16.7) | (37.5) |
| | Not Hispanic/Latino | 5 | 4 | 2 | 5 | 5 |
| | | (83.3) | (66.7) | (33.3) | (83.3) | (62.5) |
| Race [n (%)] | Asian | 2 | 1 | 1 | 0 | 0 |
| | | (33.3) | (16.7) | (16.7) | | |
| | Black or African | 2 | 1 | 1 | 3 | 3 |
| | American | (33.3) | (16.7) | (16.7) | (50.0) | (37.5) |
| | White | 2 | 4 | 4 | 3 | 4 |
| | | (33.3) | (66.7) | (66.7) | (50.0) | (50.0) |
| | Other | 0 | 0 | 0 | 0 | 1 |
| | | | | | | (12.5) |
| Weight (kg) | Mean (SD) | 86.43 | 90.48 | 88.25 | 86.72 | 88.16 |
| | | (8.379) | (9.904) | (11.190) | (9.349) | (8.539) |
| | Median | 83.40 | 91.25 | 85.95 | 85.25 | 87.15 |
| | Min, Max | 79.2, 99.2 | 77.6, 102.8 | 78.1, 108.9 | 76.7, 103.1 | 78.0, 101.8 |
| Height (cm) | Mean (SD) | 176.20 | 174.78 | 175.52 | 171.13 | 176.78 |
| | | (7.039) | (8.049) | (10.758) | (8.194) | (8.129) |
| | Median | 176.85 | 175.35 | 172.95 | 170.60 | 179.10 |
| | Min, Max | 167.4, 184.3 | 161.3, 186.4 | 163.7, 194.2 | 162.9, 185.9 | 162.0, 184.7 |
| BMI (kg/m²) | Mean (SD) | 27.83 | 29.58 | 28.62 | 29.57 | 28.21 |
| | | (2.377) | (1.824) | (1.853) | (1.900) | (1.773) |
| | Median | 27.90 | 29.70 | 28.15 | 29.80 | 28.75 |
| | Min, Max | 25.3, 31.7 | 26.7, 31.7 | 26.8, 31.6 | 25.9, 31.2 | 25.1, 30.3 |

TABLE 6
Demographic Characteristics (Safety Population) - Part 2 (Food-Effect
Assessment) (Abbreviations: BMI = body mass index; Max = maximum;
Min = minimum; N = number of subjects who received the specified
treatment; n = number of subjects/events in a category; SD = standard deviation).

| | | Period 1 | Period 2 | |
|---|---|---|---|---|
| | | NRL-1049 | NRL-1049 | |
| | | 150 mg | 150 mg | Placebo |
| | | Fasting | Fed | Combined |
| | | (N = 6) | (N = 10) | (N = 2) |
| Age (years) | Mean (SD) | 36.0 | 35.5 | 28.5 |
| | | (7.67) | (8.48) | (2.12) |
| | Median | 37.5 | 36.0 | 28.5 |
| | Min, Max | 26, 46 | 24, 50 | 27, 30 |
| Sex [n (%)] | Male | 5 | 4 | 2 |
| | | (83.3) | (40.0) | (100.0) |
| | Female | 1 | 6 | 0 |
| | | (16.7) | (60.0) | |
| Ethnicity [n (%)] | Hispanic/Latino | 4 | 5 | 1 |
| | | (66.7) | (50.0) | (50.0) |
| | Not Hispanic/Latino | 2 | 5 | 1 |
| | | (33.3) | (50.0) | (50.0) |
| Race [n (%)] | Asian | 1 | 0 | 0 |
| | | (16.7) | | |
| | Black or African | 1 | 3 | 0 |
| | American | (16.7) | (30.0) | |
| | White | 4 | 4 | 1 |
| | | (66.7) | (40.0) | (50.0) |
| | Other | 0 | 3 | 1 |
| | | | (30.0) | (50.0) |
| Weight (kg) | Mean (SD) | 88.25 | 86.24 | 87.15 |
| | | (11.190) | (9.844) | (0.636) |
| | Median | 85.95 | 82.00 | 87.15 |
| | Min, Max | 78.1, 108.9 | 77.9, 108.9 | 86.7, 87.6 |
| Height (cm) | Mean (SD) | 175.52 | 172.80 | 180.05 |
| | | (10.758) | (11.362) | (1.344) |
| | Median | 172.95 | 171.00 | 180.05 |
| | Min, Max | 163.7, 194.2 | 156.7, 194.2 | 179.1, 181.0 |
| BMI (kg/m²) | Mean (SD) | 28.62 | 28.91 | 26.90 |
| | | (1.853) | (2.075) | (0.566) |
| | Median | 28.15 | 28.85 | 26.90 |
| | Min, Max | 26.8, 31.6 | 24.1, 31.7 | 26.5, 27.3 |

### NRL-1049

Per Table 7, following single oral doses of 25 to 250 mg of NRL-1049, median time to maximum observed NRL-1049 plasma concentration (Tₘₐₓ) was reached by 0.50 to 0.75 hours in the fasting state. Over the dose range 25 to 250 mg in the fasting state, mean Cₘₐₓ ranged from 3.659 to 58.006 ng/mL, mean AUC_{0-T} ranged from 3.032 to 76.510 h*ng/mL, mean AUC_{inf} ranged from 3.806 to 78.185 h*ng/mL, and mean AUC_{0-Tmax} ranged from 1.256 to 8.728 h*ng/mL. The mean elimination half-life values ranged from 0.52 to 1.26 hours over the dose groups. The NRL-1049 mean apparent clearance (CL/F) decreased from 7420 to 3370 L/h with an increase in dose from 25 to 250 mg, while the mean apparent volume of distribution (V_{z}/F) increased from 5500 to 10800 L with an increase in dose from 25 to 150 mg, and then decreased to 4940 L for the 250-mg dose.

TABLE 7
Summary Statistics of Plasma PK Parameters of NRL-1049

| | Tₘₐₓ | Cₘₐₓ | AUC_{0-T} | AUC_{inf} | AUC_{0-Tmax} | T_{half} | CL/F | V_{z}/F |
|---|---|---|---|---|---|---|---|---|
| Cohort | (h) | (ng/mL) | (h*ng/mL) | (h*ng/mL) | (h*ng/mL) | (h) | (L/h) | (L) |
| Cohort 1: | 0.75 | 3.659 | 3.032 | 3.806 | 1.256 | 0.52 | 7420 | 5500 |
| 25 mg | (0.50-1.25) | (45.7%) | (48.6%) | (43.8%)^{a} | (73.2%)^{b} | (36.0%)^{a} | (34.5%)^{a} | (50.2%)^{a} |
| (N = 6) | | | | | | | | |
| Cohort 2: | 0.50 | 16.873 | 13.794 | 15.151 | 2.878 | 0.81 | 5350 | 6020 |
| 75 mg | (0.33-0.75) | (35.9%) | (38.6%) | (35.7%) | (49.3%)^{a} | (33.6%) | (25.8%) | (34.1%) |
| (N = 6) | | | | | | | | |
| Cohort 3: | 0.50 | 34.868 | 34.756 | 36.246 | 6.161 | 1.26 | 4820 | 9120 |
| 150 mg | (0.33-1.25) | (74.4%) | (49.2%) | (46.4%) | (80.1%) | (60.4%) | (40.5%) | (80.4%) |
| (Fasted) | | | | | | | | |
| (N = 6) | | | | | | | | |
| Cohort 3: | 0.88 | 18.478 | 42.016 | 44.722 | 6.145 | 2.05 | 3700 | 10800 |
| 150 mg | (0.33-4.00) | (64.0%) | (33.7%) | (32.6%) | (85.0%)^{c} | (31.2%) | (35.0%) | (43.6%) |
| (Fed) | | | | | | | | |
| (N = 10) | | | | | | | | |
| Cohort 4: | 0.50 | 58.006 | 76.510 | 78.185 | 8.728 | 1.03 | 3370 | 4940 |
| 250 mg | (0.50-0.75) | (40.0%) | (27.1%) | (26.0%) | (50.1%) | (19.3%) | (23.4%) | (23.2%) |
| (N = 6) | | | | | | | | |

Per Table 8, the maximum NRL-1049 plasma exposure as assessed by Cₘₐₓ under fasting conditions increased in a dose proportional manner whilst the AUC_{0-T} and AUC_{inf} increased in a greater than dose proportional manner and the AUC_{0-Tmax} increased in a dose proportional manner over the dose increase from 25 to 250 mg NRL-1049. As the dose increased in a 1:3:6:10 ratio, mean Cmax increased in a 1:5:10:16 ratio, AUC_{0-T} increased in a 1:5:11:25 ratio, AUC_{inf} increased in a 1:4:10:21 ratio and AUC_{0-Tmax} increased in a 1:2:5:7 ratio.

TABLE 8
Statistical Analysis for Dose Proportionality
of NRL-1049 PK Parameters

| | | | | 90% Confidence | | |
|---|---|---|---|---|---|---|
| | | | | Interval of Slope | | |
| | | Intercept | Slope | Lower | Upper | p- |
| Parameter | N | (α) | (β) | Bound | Bound | Value |
| Cₘₐₓ (ng/mL) | 24 | −2.478 | 1.172 | 0.955 | 1.388 | <.001 |
| AUC_{inf} (h*ng/mL) | 23 | −2.980 | 1.312 | 1.167 | 1.456 | <.001 |
| AUC_{0-T} (h*ng/mL) | 24 | −3.497 | 1.404 | 1.260 | 1.547 | <.001 |
| AUC_{0-Tmax} (h*ng/mL) | 21 | −2.621 | 0.826 | 0.490 | 1.163 | <.001 |

Per Table 7, following a single oral dose of NRL-1049 150 mg in the fed state, the NRL-1049 median Tₘₐₓ increased slightly from 0.50 to 0.88 hours, while the exposure as assessed by mean Cₘₐₓ decreased when compared to the fasting state. The exposure, as assessed by mean NRL-1049 AUC_{0-T} and AUC_{inf}, was greater in the fed state when compared to the fasting state. The mean elimination half-life was slightly increased from 0.26 to 2.05 hours in the fed state, relative to fasting state. The mean apparent clearance was greater in the fasting state whilst the mean apparent volume of distribution was greater in the fed state. Given that the evaluations of fasted versus fed are from different subject groups with a large inter-subject variability, the above exposure comparisons should be interpreted with caution.

### NRL-2017

Per Table 9, following single oral doses of 25 to 250 mg of NRL-1049, median time to maximum observed NRL-2017 plasma concentration (Tₘax) was reached by 0.88 to 1.63 hours in the fasting state. Over the dose range of 25 to 250 mg in fasting state, mean Cₘₐₓ ranged from 54.23 to 1524.35 ng/mL, mean AUC_{0-T} ranged from 477.92 to 6693.48 h*ng/mL, mean AUC_{inf} ranged from 545.54 to 7039.84 h*ng/mL and mean AUC_{0-Tmax} ranged from 35.62 to 456.89 h*ng/mL. The mean elimination half-life values ranged from 12.28 to 17.26 hours over the dose groups.

TABLE 9
Summary Statistics of Plasma PK Parameters of NRL-2017

| | Tₘₐₓ | Cₘₐₓ | AUC_{0-T} | AUC_{inf} | AUC_{0-Tmax} | T_{half} |
|---|---|---|---|---|---|---|
| Cohort | (h) | (ng/mL) | (h*ng/mL) | (h*ng/mL) | (h*ng/mL) | (h) |
| Cohort 1: 25 mg | 1.63 | 54.23 | 477.92 | 545.54 | 35.62 | 17.26 |
| (N = 6) | (1.00-1.75) | (49.8%) | (19.4%) | (14.1%) | (82.2%) | (35.5%) |
| Cohort 2: 75 mg | 1.00 | 318.00 | 1741.00 | 1871.96 | 107.53 | 15.02 |
| (N = 6) | (0.50-1.50) | (30.5%) | (22.9%) | (21.7%) | (56.8%) | (15.6%) |
| Cohort 3: 150 mg (Fasted) | 1.13 | 736.12 | 3248.21 | 3412.80 | 372.29 | 12.28 |
| (N = 6) | (0.75-1.75) | (39.0%) | (7.9%) | (7.3%) | (56.1%) | (17.1%) |
| Cohort 3: 150 mg (Fed) | 1.75 | 295.65 | 3203.83 | 3431.37 | 187.05 | 12.37 |
| (N = 10) | (1.25-6.02) | (39.1%) | (14.6%) | (13.1%) | (62.7%) | (21.4%) |
| Cohort 4: 250 mg | 0.88 | 1524.35 | 6693.48 | 7039.84 | 456.89 | 12.95 |
| (N = 6) | (0.75-1.25) | (26.7%) | (10.8%) | (9.8%) | (41.5%) | (30.0%) |

Per Table 10, the maximum NRL-2017 plasma exposure as assessed by Cₘₐₓ, AUC_{0-T}, and AUC_{inf} under fasting conditions generally increased in a greater than dose proportional manner and the AUC_{0-Tmax} increased in a dose proportional manner over the dose increase from 25 to 250 mg of NRL-1049. As the dose increased in a 1:3:6:10 ratio, mean Cₘax increased in a 1:6:14:28 ratio, AUC_{0-T} increased in a 1:4:7:14 ratio, AUC_{inf} increased in a 1:3:6:13 ratio and AUC_{0-Tmax} increased in a 1:3:10:13 ratio.

TABLE 10
Statistical Analysis for Dose Proportionality
of NRL-2017 PK Parameters

| | | | | 90% Confidence | | |
|---|---|---|---|---|---|---|
| | | | | Interval of Slope | | |
| | | Intercept | Slope | Lower | Upper | p- |
| Parameter | N | (α) | (β) | Bound | Bound | Value |
| Cₘₐₓ (ng/mL) | 24 | −0.729 | 1.459 | 1.310 | 1.609 | <.001 |
| AUC_{inf} (h*ng/mL) | 24 | 2.791 | 1.087 | 1.028 | 1.146 | <.001 |
| AUC_{0-T} (h*ng/mL) | 24 | 2.525 | 1.128 | 1.061 | 1.196 | <.001 |
| AUC_{0-Tmax} (h*ng/mL) | 24 | −0.752 | 1.246 | 0.940 | 1.552 | <.001 |

Per Table 9, following a single oral dose of NRL-1049 150 mg in the fed state, the NRL-2017 median Tₘₐₓ increased slightly from 1.13 to 1.75 hours, while the exposure as assessed by mean Cₘₐₓ decreased when compared to the fasting state. The exposure, as assessed by mean NRL-2017 AUC_{0-T} and AUC_{inf}, remained consistent in the fed state when compared to the fasting state. The mean elimination half-life also remained consistent in the fed state (12.37 hours) when compared to the fasting state (12.28 hours). Given that the evaluations of fasted versus fed are from different subject groups with a large inter-subject variability, the above exposure comparisons should be interpreted with caution.

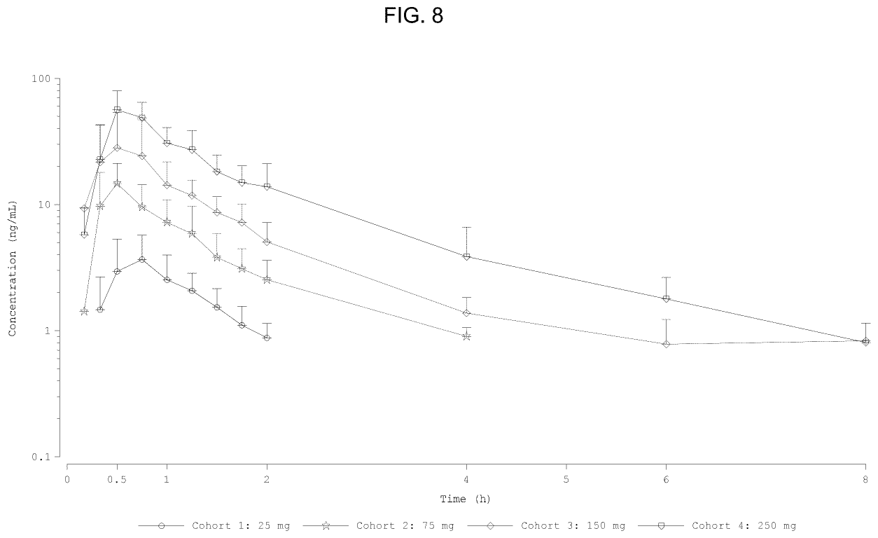

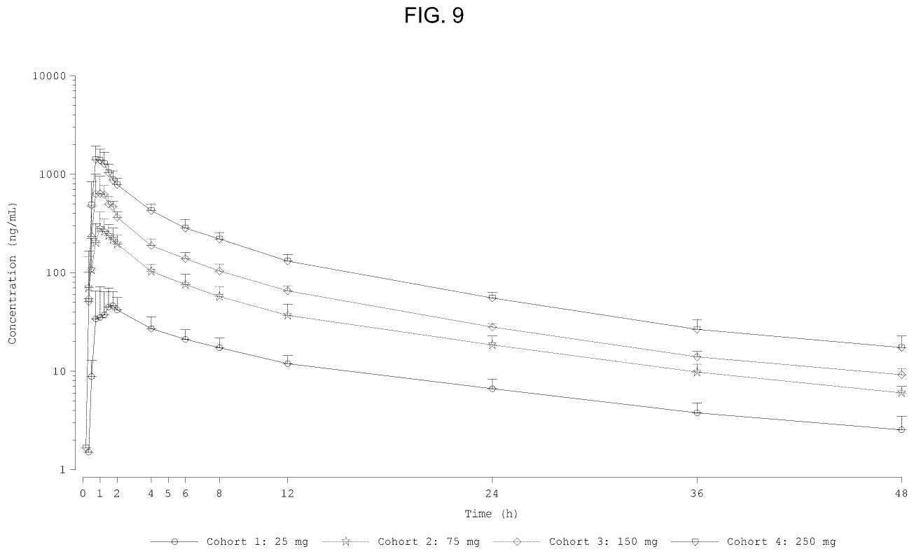

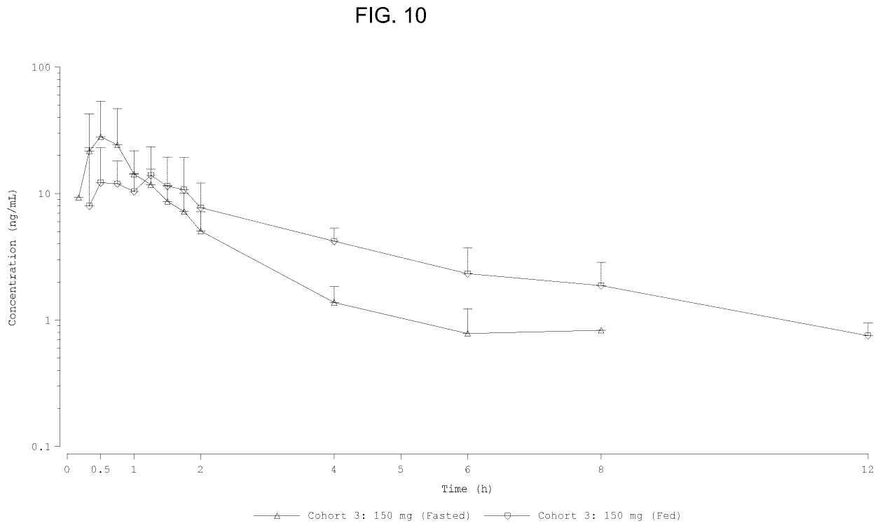

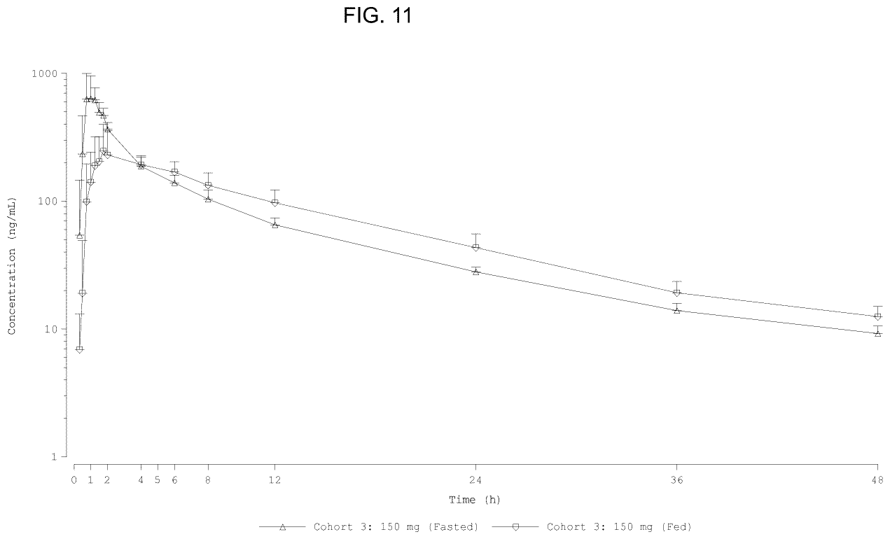

FIG. 8 shows the mean (+SD) Plasma Concentration-Time Profiles of NRL-1049 Under Fasted Condition—Semi-Log Scale. FIG. 9 shows the mean (+SD) Plasma Concentration-Time Profiles of NRL-2017 Under Fasted Condition—Semi-Log Scale. FIG. 10 shows the mean (+SD) Plasma Concentration-Time Profiles of NRL-1049 Under Fasted and Fed Conditions—Semi-Log Scale. FIG. 11 shows the mean (+SD) Plasma Concentration-Time Profiles of NRL-2017 Under Fasted and Fed Conditions—Semi-Log Scale.

### Conclusions

Following single oral doses of NRL-1049 to healthy subjects, all subjects were exposed to NRL-1049. Mean NRL-1049 concentrations were quantifiable up to 2 hours for the 25-mg dose, 4 hours for the 75-mg dose, and 8 hours postdose for the 150- and 250-mg doses in the fasting state. Mean NRL-1049 concentrations were quantifiable up to 12 hours postdose in the fed state for the 150-mg dose. Mean NRL-2017 concentrations were quantifiable up to 48 hours postdose for all dose levels of 25 to 250 mg in the fasting and for 150 mg in the fed state.

Following single oral doses of 25 to 250 mg of NRL-1049 in the fasting state, plasma NRL-1049 exposure as assessed by Cₘₐₓ increased in a dose proportional manner while the exposure as assessed by AUC_{0-T} and AUC_{inf} increased in a greater than dose proportional manner and AUC_{0-Tmax} increased in a dose proportional manner. As the dose increased in a 1:3:6:10 ratio, mean Cₘax increased in a 1:5:10:16 ratio, AUC_{0-T} increased in a 1:5:11:25 ratio, AUC_{inf} increased in a 1:4:10:21 ratio and AUC_{0-Tmax} increased in a 1:2:5:7 ratio. The mean elimination half-life values ranged from 0.52 to 1.26 hours.

Following single oral doses of 25 to 250 mg of NRL-1049 in the fasting state, plasma NRL-2017 exposure as assessed by Cₘₐₓ, AUC_{0-T}, and AUC_{inf} generally increased in a greater than dose proportional manner and AUC_{0-Tmax} increased in a dose proportional manner. As the dose increased in a 1:3:6:10 ratio, mean Cₘax increased in a 1:6:14:28 ratio, AUC_{0-T} increased in a 1:4:7:14 ratio, AUC_{inf} increased in a 1:3:6:13 ratio and AUC_{0-Tmax} increased in a 1:3:10:13 ratio. The mean elimination half-life values ranged from 12.28 to 17.26 hours.

Food effect was analyzed descriptively for the NRL-1049 150-mg dose group as different subjects participated in the fasting and fed states. Plasma NRL-1049 exposures as assessed by Cₘₐₓ decreased following the fed state while exposures as assessed by AUC_{0-T} and AUC_{inf} increased. The NRL-1049 mean half-life values were longer in the fed state. Plasma NRL-2017 exposures as assessed by Cmax decreased following the fed state while exposures as assessed by AUC_{0-T} and AUC_{inf} remained consistent. The NRL-2017 mean half-life values were also consistent between the fed and fasting states.

### SAD Assessment

Single oral dose administration of NRL-1049 under fasting state was generally well tolerated in healthy subjects. No deaths or SAEs occurred during Part 1 and no subject was discontinued due to TEAEs. In Part 1 (SAD), 9 of the 32 subjects (28.1%) experienced a total of 13 TEAEs following administration of a single oral dose of NRL-1049 under fasting state, with 10 of the 13 TEAEs (76.9%) deemed related to study treatment. No TEAEs were noted in the placebo combined group. A gradual increase in the proportion of subjects experiencing at least 1 TEAE was noted with increasing NRL-1049 dose levels, with the highest proportion observed with NRL 1049 250 mg (66.7%). A similar profile was observed for treatment-related TEAEs. The majority of TEAEs were of Grade 2 (61.5%) followed by Grade 1 (30.8%). One TEAE (7.7%) of Grade 3 (syncope) was experienced after receiving NRL 1049 250 mg and was deemed related to study treatment. None of the subjects experienced TEAEs of Grade 4 or higher. All TEAEs were resolved at the end of the study. The most common TEAE was dizziness, experienced by 2 of the 6 subjects (33.3%) receiving NRL-1049 150 mg, and 1 of 6 subjects (16.7%) each with NRL-1049 75 mg and NRL 1049 250 mg. A potential trend observed in reduction of blood pressure with increasing doses appears to correlate with the overall profile of TEAEs observed at the highest dose level with NRL-1049 250 mg (dizziness, headache, syncope, and orthostatic hypotension). This reduction in blood pressure was deemed to be clinically relevant by the Investigator, warranting potential future investigation. No CS abnormal findings in laboratory parameters, vital signs, ECGs, physical examination, or cardiac telemetry were noted in Part 1.

### Food-Effect Assessment

Single oral dose administration of NRL-1049 150 mg under fasting and fed state were generally well tolerated in healthy subjects. No deaths or SAEs occurred during Part 2 and no subject was discontinued due to TEAEs. Following a single oral dose of NRL-1049 150 mg under fasting and fed state, 6 of the 15 subjects (40.0%) experienced a total of 6 TEAEs, with 5 TEAEs deemed related to study treatment. In the placebo combined group, 1 of the 2 subjects experienced 1 TEAE that was deemed related to study treatment. The proportion of subjects with at least 1 TEAE was similar when NRL-1040 150 mg was administered under fasting (33.3%) and fed (40.0%) state. Similar proportions were also noted for subjects with treatment-related TEAEs under fasting (33.3%) and fed (30.0%) state, suggesting there is no food effect on the AE profile of NRL-1049 at a dose level of 150 mg. The TEAEs reported were of Grade 1/mild (57.1%) and Grade 2/moderate (42.9%). None of the subjects experienced TEAEs of Grade 3/severe and higher. All TEAEs were resolved at the end of the study. The most frequently reported TEAEs were dizziness and headache. Dizziness was experienced by 2 of the 6 subjects (33.3%) with NRL-1049 150 mg under fasting state and headache was experienced by 2 of the 10 subjects (20.0%) with NRL-1049 150 mg under fed state. No CS abnormal findings in laboratory parameters, vital signs, ECGs, physical examination, or cardiac telemetry were noted in Part 2.

### DISCUSSION AND OVERALL CONCLUSIONS

The objectives of this first-in-human study were to determine the MTD, collect data on the safety and tolerability of NRL-1049, assess the PK of NRL-1049 and its active metabolite (NRL-2017), and evaluate the effect of food on the PK of NRL-1049 following oral administration of NRL 1049 in healthy male and female volunteers. Following single oral doses of NRL 1049 to healthy subjects, all subjects were exposed to NRL 1049. Following single oral doses of 25 to 250 mg of NRL 1049 in the fasting state, plasma NRL 1049 exposure as assessed by Cmax increased in a dose proportional manner while the AUC0-T and AUCinf increased in a greater than dose proportional manner and AUC0-Tmax increased in a dose proportional manner. As the dose increased in a 1:3:6:10 ratio, mean Cmax increased in a 1:5:10:16 ratio, AUC0-T increased in a 1:5:11; 25 ratio, AUCinf increased in a 1:4:10:21 ratio and AUC0-Tmax increased in a 1:2:5:7 ratio. Following single oral doses of 25 to 250 mg of NRL 1049 in the fasting state, plasma NRL 2017 exposure as assessed by Cmax, AUC0-T and AUCinf generally increased in a greater than dose proportional manner and AUC0-Tmax increased in a dose proportional manner. As the dose increased in a 1:3:6:10 ratio, mean Cmax increased in a 1:6:14:28 ratio, AUC0-T increased in a 1:4:7:14 ratio, AUCinf increased in a 1:3:6:13 ratio and AUC0-Tmax increased in a 1:3:10:13 ratio. As analyzed descriptively, plasma NRL 1049 exposures as assessed by mean Cmax decreased following the fed state while the exposure as assessed by AUC0-T and AUCinf increased. The NRL 1049 mean half-life values were longer in the fed state. Plasma NRL 2017 exposures as assessed by mean Cmax decreased following the fed state while the exposure as assessed by AUC0-T and AUCinf remained consistent. The NRL 2017 mean half-life values were also consistent between the fed and fasting states. Overall, NRL-1049 was generally well tolerated in healthy subjects following a single oral dose administration. There were no deaths, SAEs, or Grade 4/5 TEAEs, and none of the subjects were withdrawn due to AEs. The majority of TEAEs (92.3%) were Grade 1 or Grade 2; 1 subject (7.7%) experienced one Grade 3 TEAE (syncope) following administration of NRL 1049 250 mg, the top dose evaluated in this study. The results in this study established tolerability of NRL 1049 at 150 mg, therefore the MTD for this study was determined to be the 150 mg dose. However, intermediate dose levels between 150 and 250 mg were not explored.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A method of treating cavernous angioma lesions with symptomatic hemorrhage in a subject comprising: administering to the subject a therapeutically effective amount of a compound of formula (II): 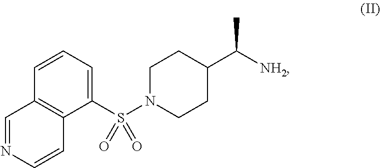 [SMILES: C[C@@H](N)C1CCN(S(=O)(=O)c2cccc3cnccc23)CC1]
or a pharmaceutically acceptable salt thereof;
wherein administering the therapeutically effective amount of the compound of formula (II), or a pharmaceutically acceptable salt thereof results in a Cmax of between about 3 ng/ml and about 60 ng/ml; and
wherein a Tmax is achieved of between about 0.5 hours and about 1 hour of administering the compound of formula (II), or a pharmaceutically acceptable salt thereof.

2. The method of claim 1, wherein the salt is an adipate salt of the compound of formula (II) or a pharmaceutically acceptable salt thereof.

3. The method of claim 1, wherein the therapeutically effective amount of the compound of formula (II), or a pharmaceutically acceptable salt thereof is from about 25 mg to about 250 mg.

4. The method of claim 1, wherein the therapeutically effective amount of the compound of formula (II), or a pharmaceutically acceptable salt thereof is an amount sufficient to result in a plasma concentration of between about 0.1 ng/ml and about 2000 ng/ml of a compound of Formula (IV):

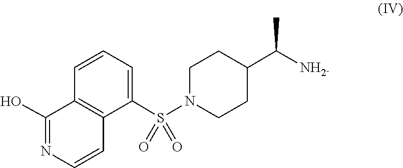 [SMILES: C[C@@H](N)C1CCN(S(=O)(=O)c2cccc3c(O)nccc23)CC1]

5. The method of claim 4, wherein the plasma concentration of the compound of Formula (IV) is maintained for at least 48 hours following administration of the compound of formula (II), or a pharmaceutically acceptable salt thereof.

6. The method of claim 1, wherein the therapeutically effective amount of the compound of formula (II), or a pharmaceutically acceptable salt thereof is an amount sufficient to result in a Cmax of between about 50 ng/ml and about 1600 ng/ml of a compound of Formula (IV):

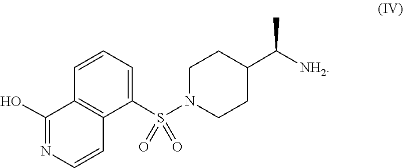 [SMILES: C[C@@H](N)C1CCN(S(=O)(=O)c2cccc3c(O)nccc23)CC1]

7. The method of claim 1, wherein the therapeutically effective amount of the compound of formula (II), or a pharmaceutically acceptable salt thereof is an amount sufficient to achieve a Tmax for a compound of Formula (IV): 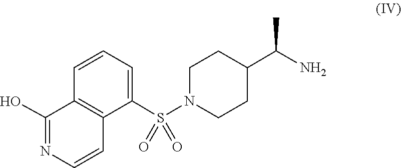 [SMILES: C[C@@H](N)C1CCN(S(=O)(=O)c2cccc3c(O)nccc23)CC1]
between about 1 hour and about 1.75 hours of administering the compound of formula (II), or a pharmaceutically acceptable salt thereof.

8. The method of claim 1, wherein the compound of formula (II) or a pharmaceutically acceptable salt thereof is administered orally.

9. The method of claim 1, wherein the subject is fasted when administering to the subject the therapeutically effective amount of the compound of formula (II), or a pharmaceutically acceptable salt thereof.

10. The method of claim 1, wherein the subject is fed when administering to the subject the therapeutically effective amount of the compound of formula (II), or a pharmaceutically acceptable salt thereof.

11. The method of claim 1, wherein the compound of formula (II), or a pharmaceutically acceptable salt thereof is administered parenterally.

12. The method of claim 1, wherein the compound of formula (II), or a pharmaceutically acceptable salt thereof is administered once a day.

13. The method of claim 1, wherein the compound of formula (II), or a pharmaceutically acceptable salt thereof is administered twice a day.

14. The method of claim 1, wherein the compound of formula (II), or a pharmaceutically acceptable salt thereof is administered three times a day.

15. The method of claim 1, wherein the subject is diagnosed with a sporadic cavernous angioma lesion.

16. The method of claim 1, wherein the subject is diagnosed with a familial cavernous angioma lesion caused by germline loss-of-function mutations in a gene selected from the group consisting of CCM1 (KRIT1), CCM2 (malcavernin, MGC4607), or CCM3 (PDCD10).

17. The method of claim 1, wherein the subject has multiple cavernous angioma lesions.

18. The method of claim 1, wherein the cavernous angioma lesion has not been resected prior to administration of the compound of formula (II), or a pharmaceutically acceptable salt thereof.

19. The method of claim 1, Wherein the cavernous angioma lesion occurs in the brainstem.

20. The method of claim 1, wherein the cavernous angioma lesion is a Stage 1 lesion.

21. The method of claim 1, wherein the cavernous angioma lesion is a Stage 2 lesion.

22. The method of claim 1, wherein the subject has had a symptomatic first hemorrhage prior to administration of the compound of formula (II), or a pharmaceutically acceptable salt thereof.

23. The method of claim 1, wherein the subject has had at least one subsequent hemorrhage/rebleed following a symptomatic first hemorrhage prior to administration of the compound of formula (II), or a pharmaceutically acceptable salt thereof.

24. The method of claim 1, wherein administering to the subject the therapeutically effective amount of the compound of formula (II), or a pharmaceutically acceptable salt thereof results in a decrease in the number of cavernous angioma lesions.

25. The method of claim 1, wherein administering to the subject the therapeutically effective amount of the compound of formula (II), or a pharmaceutically acceptable salt thereof results in a decrease in the number of subsequent hemorrhages/rebleeds.

26. The method of claim 1, wherein administering to the subject the therapeutically effective amount of the compound of formula (II), or a pharmaceutically acceptable salt thereof results in an improvement in blood brain barrier permeability measured by MRI imaging using the Dynamic Contrast Enhanced Quantitative Perfusion (DCEQP), Quantitative Susceptibility Mapping (QSM) or a combination thereof.

27. The method of claim 1, wherein administering to the subject the therapeutically effective amount of the compound of formula (II), or a pharmaceutically acceptable salt thereof results in increased lesion stability measured by Quantitative Susceptibility Mapping (QSM).

28. The method of claim 27, wherein a decrease of about 6% less in mean lesional Quantitative Susceptibility Mapping (QSM) is indicative of increased lesional stability.

29. The method of claim 27, wherein increased lesional stability is indicative of a lack of growth or symptomatic hemorrhages/rebleeds.

30. The method of claim 1, wherein administering to the subject the therapeutically effective amount of the compound of formula (II), or a pharmaceutically acceptable salt thereof results in an improvement in subjects' modified Rankin Scale, Mini-Mental State Examination, Euro-Quality of Life [QoL]5D, Euro-QoL, Visual Analogue Scale, Neuro-QoL, or any combination thereof.

31. The method of claim 1, wherein administering to the subject the therapeutically effective amount of the compound of formula (II), or a pharmaceutically acceptable salt thereof results in a decrease in one or more symptoms of a symptomatic hemorrhage selected from double vision, facial droop, balance problems, difficulty swallowing, pupil and vision changes, nausea, projectile vomiting, headache, confusion, impaired consciousness and combinations thereof.

32. A method of treating cavernous angioma lesions in a subject comprising: administering to the subject the therapeutically effective amount of the compound of formula (II): 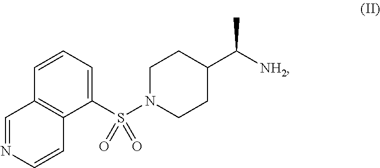 [SMILES: C[C@@H](N)C1CCN(S(=O)(=O)c2cccc3cnccc23)CC1]
or a pharmaceutically acceptable salt thereof;
wherein administering the therapeutically effective amount of the compound of formula (II), or a pharmaceutically acceptable salt thereof results in a Cmax of between about 3 ng/ml and about 60 ng/ml; and
wherein a Tmax is achieved of between about 0.5 hours and about 1 hour of administering the compound of formula (II), or a pharmaceutically acceptable salt thereof.

33. The method of claim 32, wherein the salt is an adipate salt of the compound of formula (II), or a pharmaceutically acceptable salt thereof.

34. The method of claim 32, wherein the therapeutically effective amount of the compound of formula (II) is from about 25 mg to about 250 mg.

35. The method of claim 32, wherein the therapeutically effective amount of the compound of formula (II) is an amount sufficient to result in a plasma concentration of between about 0.1 ng/ml and about 2000 ng/ml of a compound of Formula (IV):

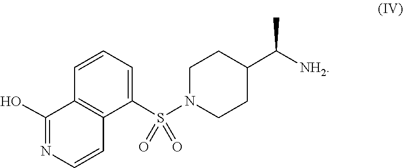 [SMILES: C[C@@H](N)C1CCN(S(=O)(=O)c2cccc3c(O)nccc23)CC1]

36. The method of claim 35, wherein the plasma concentration of the compound of Formula (IV) is maintained for at least 48 hours following administration of the compound of formula (II), or a pharmaceutically acceptable salt thereof.

37. The method of claim 32, wherein the therapeutically effective amount of the compound of formula (II), or a pharmaceutically acceptable salt thereof is an amount sufficient to result in a Cmax of between about 50 ng/ml and about 1600 ng/ml of a compound of Formula (IV):

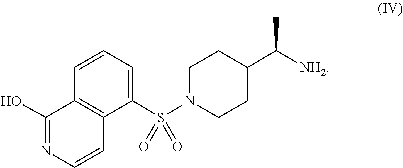 [SMILES: C[C@@H](N)C1CCN(S(=O)(=O)c2cccc3c(O)nccc23)CC1]

38. The method of claim 32, wherein the therapeutically effective amount of the compound of formula (II), or a pharmaceutically acceptable salt thereof is an amount sufficient to achieve a Tmax of a compound of Formula (IV): 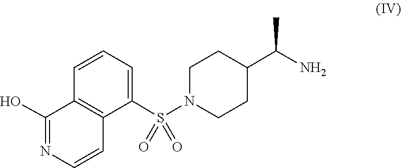 [SMILES: C[C@@H](N)C1CCN(S(=O)(=O)c2cccc3c(O)nccc23)CC1]
between about 1 hour and about 1.75 hours of administering the compound of formula (II), or a pharmaceutically acceptable salt thereof.

39. The method of claim 32, wherein the compound of formula (II), or a pharmaceutically acceptable salt thereof is administered orally.

40. The method of claim 32, wherein the subject is fasted when administering to the subject the therapeutically effective amount of the compound of formula (II), or a pharmaceutically acceptable salt thereof.

41. The method of claim 32, wherein the subject is fed when administering to the subject the therapeutically effective amount of the compound of formula (II), or a pharmaceutically acceptable salt thereof.

42. The method of claim 32, wherein the compound of formula (II), or a pharmaceutically acceptable salt thereof is administered once a day.

43. The method of claim 32, wherein the compound of formula (II), or a pharmaceutically acceptable salt thereof is administered twice a day.

44. The method of claim 32, wherein the compound of formula (II), or a pharmaceutically acceptable salt thereof is administered three times a day.

45. The method of claim 32, wherein the subject is diagnosed with a sporadic cavernous angioma lesion.

46. The method of claim 32, wherein the subject is diagnosed with a familial cavernous angioma lesion caused by germline loss-of-function mutations in a gene selected from the group consisting of CCM1 (KRIT1), CCM2 (malcavernin, MGC4607), CCM3 (PDCD10) or any combination thereof.

47. The method of claim 32, wherein the subject has multiple cavernous angioma lesions.

48. The method of claim 32, wherein the cavernous angioma lesion has not been resected prior to administration of the compound of formula (II) or a pharmaceutically acceptable salt thereof.

49. The method of claim 32, wherein the cavernous angioma lesion occurs in the brainstem.

50. The method of claim 32, wherein the cavernous angioma lesion is a Stage 1 lesion.

51. The method of claim 32, wherein the cavernous angioma lesion is a Stage 2 lesion.

52. The method of claim 32, wherein administering to the subject the therapeutically effective amount of the compound of formula (II), or a pharmaceutically acceptable salt thereof results in a decrease in the number of cavernous angioma lesions.

53. The method of claim 32, wherein administering to the subject the therapeutically effective amount of the compound of formula (II), or a pharmaceutically acceptable salt thereof results in an improvement in blood brain barrier permeability measured by MRI imaging using the Dynamic Contrast Enhanced Quantitative Perfusion (DCEQP), Quantitative Susceptibility Mapping (QSM) or a combination thereof.

54. The method of claim 32, wherein administering to the subject the therapeutically effective amount of the compound of formula (II), or a pharmaceutically acceptable salt thereof results in increased lesion stability measured by Quantitative Susceptibility Mapping (QSM).

55. The method of claim 54, wherein a decrease of about 6% less in mean lesional Quantitative Susceptibility Mapping (QSM) is indicative of increased lesional stability.

56. The method of claim 54, wherein increased lesional stability is indicative of a lack of growth, symptomatic hemorrhages/rebleeds or a combination thereof.

57. The method of claim 31, wherein administering to the subject the therapeutically effective amount of the compound of formula (II), or a pharmaceutically acceptable salt thereof results in an improvement in subjects' modified Rankin Scale, Mini-Mental State Examination, Euro-Quality of Life [QoL]5D, Euro-QoL, Visual Analogue Scale, Neuro-QoL, or any combination thereof.

58. The method of claim 31, wherein administering to the subject the therapeutically effective amount of the compound of formula (II), or a pharmaceutically acceptable salt thereof results in a decrease in one or more symptoms of cavernous angioma lesions.

59. The method of claim 58, wherein the one or more symptoms of cavernous angioma lesions are selected from seizures, neurological deficits, headache, fatigue, weakness, tingling, numbness, pain, bladder issues, bowel issues, respiratory distress, and a combination thereof.

60. The method of claim 59, wherein the seizure is selected from focal onset seizures, generalized onset seizures, and a combination thereof.

61. The method of claim 59, wherein the neurological deficits are selected from ataxia, speech and swallowing difficulties, facial paralysis, vision and auditory problems, diaphragmatic spasms, and breathing difficulties, and a combination thereof.
